(19) United States
(12) Patent Application Publication
(10) Pub. No.: US 2023/0159606 A1
(43) Pub. Date: May 25, 2023

(54) PHARMACEUTICAL COMPOSITIONS FOR GLUCAGON AND GLP-1 CO-AGONIST PEPTIDES

(71) Applicant: MEDIMMUNE LIMITED, CAMBRIDGE (GB)

(72) Inventor: ANA LUCIA GOMES DOS SANTOS, CAMBRIDGE (GB)

(21) Appl. No.: 17/618,951

(22) PCT Filed: Jun. 30, 2020
(86) PCT No.: PCT/EP2020/068411

§ 371 Date: Dec. 14, 2021

Related U.S. Application Data
(60) Provisional application No. 62/869,263, filed on Jul. 1, 2019.

(51) Int. Cl. C07K 14/605; A61K 47/02; A61K 47/10; A61K 47/26

(52) U.S. Cl. CPC C07K 14/605; A61K 47/02; A61K 47/10; A61K 47/26

(57) ABSTRACT

The present invention provides formulations for parenteral administration of GLP-1/Glucagon agonist peptides, methods of making such formulations, and methods of treatment using such formulations.

### REFERENCE TO SEQUENCE LISTING SUBMITTED ELECTRONICALLY

The content of the electronically submitted sequence listing in ASCII text file (Name GLPGG-300-WO-PCT_ST25.txt; Size: 1,840 bytes; and Date of Creation: Jun. 22, 2020) filed with the application is incorporated herein by reference in its entirety.

### BACKGROUND

### Field of the Invention

The present disclosure relates to pharmaceutical compositions for administration of glucagon and GLP-1 co-agonist peptides and methods of administering the same.

### Background

Obesity is a major and growing health problem worldwide. It is associated with many life-threatening diseases such as cardiovascular disease, renal disease, hypertension, stroke, infertility, respiratory dysfunction, and type 2 diabetes.

Glucagon and glucagon-like peptide-1 (GLP-1) derive from pre-proglucagon, a 158 amino acid precursor polypeptide that is processed in different tissues to form a number of different proglucagon-derived peptides, including glucagon, glucagon-like peptide-1 (GLP-1), glucagon-like peptide-2 (GLP-2), and oxyntomodulin (OXM), that are involved in a wide variety of physiological functions, including glucose homeostasis, insulin secretion, gastric emptying, and intestinal growth, as well as the regulation of food intake. Glucagon is a 29-amino acid peptide that corresponds to amino acids 33 through 61 of proglucagon (53 to 81 of preproglucagon), while GLP-1 is produced as a 37-amino acid peptide that corresponds to amino acids 72 through 108 of proglucagon (92 to 128 of preproglucagon).

Glucagon is produced by the pancreas and interacts with the glucagon receptor (“glucR”). Glucagon acts in the liver to raise blood glucose via gluconeogenesis and glycogenolysis. When blood glucose begins to fall, glucagon signals the liver to break down glycogen and release glucose, causing blood glucose levels to rise toward a normal level.

GLP-1 has different biological activities compared to glucagon. It is secreted from gut L cells and binds to the GLP-1 receptor. Its activities include stimulation of insulin synthesis and secretion, inhibition of glucagon secretion, and inhibition of food intake. GLP-1(7-36) amide or GLP-1(7-37) acid are biologically active forms of GLP-1, that demonstrate essentially equivalent activity at the GLP-1 receptor.

Both glucagon and GLP-1, acting as agonists at their respective receptors, have been shown to be effective in weight loss. Certain GLP-1 analogs are being sold or are in development for treatment of obesity including, e.g., Liraglutide (VICTOZA® from Novo Nordisk) and Exenatide (Byetta® from Eli Lilly/Amylin). Glucagon/GLP-1 agonist peptides have also been disclosed in WO 2014/091316.

While some therapies are available for the control of blood glucose, none currently achieves substantial weight loss, which remains a significant unmet need for patients. Fifty percent of patients progress from oral monotherapy for glucose control (usually with metformin) to initiation of insulin within 10 years, often taking multiple oral combination therapies before initiating insulin. The use of insulin exacerbates weight gain, which can be as great as 6 kilograms (kg) in the first year after starting insulin therapy. This weight gain can lead to increased insulin resistance, which is associated with hypertension, dyslipidemia, and an increased risk of major adverse cardiovascular events. With respect to reducing insulin resistance, significant weight loss (>5%) is the optimal intervention to reduce insulin resistance, although this can only be achieved reliably at present through intensive dietary and lifestyle interventions and/or bariatric surgery. There remains a need for pharmaceutical compositions for administering GLP-1/Glucagon agonist peptides.

### BRIEF SUMMARY OF THE INVENTION

MEDI0382 is a synthetic peptide containing 30 amino acids with a C16 fatty acid (palmitic acid). MEDI0382 combines both glucagon-like peptide-1 (GLP-1) and glucagon receptor co-agonism activity. The combination of GLP-1 and glucagon activity can cause significant weight loss and lead to improvement in glycemic control and lipid profiles. In order to elicit the dual agonist response, certain amino acids were structurally arranged to make the whole peptide safe and efficacious. This arrangement resulted in challenges to the stability of the formulation. In addition to structural liabilities, the addition of preservative introduced additional constraints to solution stability. The preservatives were included to formulate MEDI0382 as a multi-dose formulation for patient conveniences. Extensive formulation development was carried out to identify suitable solution conditions to support formulation stability while maintaining safety. The selection of solution pH, buffer strength, and preservative were used to generate multi-dose formulations.

The MEDI0382 multi-dose formulations advantageously allows for two different drug product presentations, so that patients can begin treatment using MEDI0382 at 1 mg/mL (a “titration dose”) and switch to 5 mg/mL (a “maintenance dose”). The use of a titration dose prior to the maintenance dose can reduce side effects.

The pharmaceutical compositions comprising GLP-1/Glucagon agonist peptides (e.g., MEDI0382) provided herein in order to achieve compositions that avoid higher order aggregates and achieve long term (e.g., 2-year) stability.

Provided herein are pharmaceutical compositions for the administration of GLP-1/Glucagon agonist peptides (e.g., MEDI0382).

In certain embodiments, a pharmaceutical composition comprises a peptide comprising SEQ ID NO:4 (MEDI0382) and the pH of the composition is about 8.1.

In certain embodiments, a pharmaceutical composition comprises a peptide comprising SEQ ID NO:4 (MEDI0382) and sorbitol.

In certain embodiments, a pharmaceutical composition comprises a peptide comprising SEQ ID NO:4 (MEDI0382) and meta-cresol.

In certain embodiments, the pH of the composition is at least 7.9. In certain embodiments, the pH of the composition is about 7.9 to about 8.4. In certain embodiments, the pH of the composition is about 8.1.

In certain embodiments, the composition comprises a pH-adjusting agent. In certain embodiments, the composition comprises sodium hydroxide. In certain embodiments, the composition comprises sodium hydroxide at a concentration sufficient to make the pH of the composition at least 7.9. In certain embodiments, the composition comprises sodium hydroxide at a concentration sufficient to make the pH of the composition about 7.9 to about 8.4. In certain embodiments, the composition comprises sodium hydroxide at a concentration sufficient to make the pH of the composition about 8.1.

In certain embodiments, the composition comprises a tonicity agent. In certain embodiments, the tonicity agent is sorbitol, mannitol, or propylene glycol. In certain embodiments, the composition comprises sorbitol. In certain embodiments, the concentration of sorbitol is about 190 mM to about 250 mM. In certain embodiments, the concentration of sorbitol is about 220 mM. In certain embodiments, the concentration of sorbitol is 220.3 mM. In certain embodiments, the concentration of sorbitol is about 35 mg/mL to about 45 mg/mL. In certain embodiments, the concentration of sorbitol is about 40 mg/mL to about 41 mg/mL. In certain embodiments, the concentration of sorbitol is 40.13 mg/mL.

In certain embodiments, the composition comprises an antimicrobial agent. In certain embodiments, the antimicrobial agent is meta-cresol or phenol. In certain embodiments, the composition comprises meta-cresol. In certain embodiments, the concentration of meta-cresol is about 0.27% w/v to about 0.45% w/v. In certain embodiments, the concentration of meta-cresol is about 0.31% w/v. In certain embodiments, the concentration of meta-cresol is about 25 mM to about 30 mM. In certain embodiments, the concentration of meta-cresol is about 28.6 mM. In certain embodiments, the concentration of meta-cresol is about 0.4% w/v. In certain embodiments, the concentration of meta-cresol is about 2.7 mg/ml to about 4.5 mg/ml. In certain embodiments, the concentration of meta-cresol is about 3.1 mg/ml. In certain embodiments, the concentration of meta-cresol is about 4 mg/ml.

In certain embodiments, the composition comprises a buffer. In certain embodiments, the buffer is sodium phosphate or TRIS.

In certain embodiments, the composition comprises sodium phosphate. In certain embodiments, the concentration of sodium phosphate is about 5 mM to about 25 mM.

In certain embodiments, the concentration of sodium phosphate is about 20 mM. In certain embodiments, the concentration of sodium phosphate is 20 mM. In certain embodiments, the concentration of sodium phosphate is 20.1 mM. In certain embodiments, the sodium phosphate comprises sodium phosphate monobasic monohydrate and sodium phosphate dibasic heptahydrate. In certain embodiments, the concentration of sodium phosphate monobasic monohydrate is about 1 mM and the concentration of sodium phosphate dibasic heptahydrate is about 19 mM. In certain embodiments, the concentration of sodium phosphate monobasic monohydrate is 1 mM and the concentration of sodium phosphate dibasic heptahydrate is 19 mM. In certain embodiments, the concentration of sodium phosphate monobasic monohydrate is 1 mM and the concentration of sodium phosphate dibasic heptahydrate is 19.1 mM.

In certain embodiments, the concentration of sodium phosphate is about 10 mM. In certain embodiments, the sodium phosphate is sodium phosphate dibasic heptahydrate.

In certain embodiments, the concentration of sodium phosphate is about 1 mg/mL to about 10 mg/mL. In certain embodiments, the concentration of sodium phosphate is about 5.25 mg/mL. In certain embodiments, the sodium phosphate comprises sodium phosphate monobasic monohydrate and sodium phosphate dibasic heptahydrate. In certain embodiments, the concentration of sodium phosphate monobasic monohydrate is about 0.13 mg/mL and the concentration of sodium phosphate dibasic heptahydrate is about 5.12 mg/mL. In certain embodiments, the concentration of sodium phosphate is about 2.68 mg/mL. In certain embodiments, the sodium phosphate is sodium phosphate dibasic heptahydrate.

In certain embodiments, the pharmaceutical composition does not comprise sodium phosphate. In certain embodiments, the pharmaceutical composition does not contain lysine, trehalose, sucrose, magnesium chloride, histidine, arginine, and/or glutamic acid.

In certain embodiments, the concentration of the peptide comprising SEQ ID NO:4 (MEDI0382) is about 0.5 mg/mL to about 5 mg/mL. In certain embodiments, the concentration of the peptide comprising SEQ ID NO:4 (MEDI0382) is about 1 mg/mL. In certain embodiments, the concentration of the peptide comprising SEQ ID NO:4 (MEDI0382) is about 2 mg/mL. In certain embodiments, the concentration of the peptide comprising SEQ ID NO:4 (MEDI0382) is about 5 mg/mL.

In certain embodiments, a pharmaceutical composition comprises about 0.5 mg/mL to about 5 mg/mL of a peptide comprising SEQ ID NO:4 (MEDI0382), about 190 mM to about 250 mM sorbitol, about 5 mM to about 25 mM sodium phosphate, and about 0.27% w/v to about 0.45% w/v meta-cresol, and the pH of the pharmaceutical composition is about 7.9 to about 8.4.

In certain embodiments, a pharmaceutical composition comprises about 0.5 mg/mL to about 5 mg/mL (e.g., about 1 mg/mL, about 2 mg/mL, or about 5 mg/mL) of a peptide comprising SEQ ID NO:4 (MEDI0382), about 220.3 mM sorbitol, about 20.1 mM sodium phosphate, and about 0.31% w/v meta-cresol, and the pH of the pharmaceutical composition is about 8.1. In certain embodiments, a pharmaceutical composition comprises about 0.5 mg/mL to about 5 mg/mL (e.g., about 1 mg/mL, about 2 mg/mL, or about 5 mg/mL) of a peptide comprising SEQ ID NO:4 (MEDI0382), about 220.3 mM sorbitol, about 20 mM sodium phosphate, and about 0.31% w/v meta-cresol, and the pH of the pharmaceutical composition is about 8.1. In certain embodiments, the sodium phosphate comprises sodium phosphate monobasic monohydrate and sodium phosphate dibasic heptahydrate.

In certain embodiments, a pharmaceutical composition comprises about 0.5 mg/mL to about 5 mg/mL (e.g., about 1 mg/mL, about 2 mg/mL, or about 5 mg/mL) of a peptide comprising SEQ ID NO:4 (MEDI0382), about 220.3 mM sorbitol, about 10 mM sodium phosphate, and about 0.31% w/v meta-cresol, and the pH of the pharmaceutical composition is about 8.1. In certain embodiments, the sodium phosphate is sodium phosphate dibasic heptahydrate.

In certain embodiments, a pharmaceutical composition comprises 0.5 mg/mL to about 5 mg/mL (e.g., about 1 mg/mL, about 2 mg/mL, or about 5 mg/mL) of a peptide comprising SEQ ID NO:4 (MEDI0382), about 220 mM sorbitol, about 20 mM sodium phosphate, and about 0.31% w/v meta-cresol, and the pH of the pharmaceutical composition is about 8.1. In certain embodiments, the sodium phosphate comprises sodium phosphate monobasic monohydrate and sodium phosphate dibasic heptahydrate.

In certain embodiments, a pharmaceutical composition comprises about 0.5 mg/mL to about 5 mg/mL (e.g., about 1 mg/mL, about 2 mg/mL, or about 5 mg/mL) of a peptide comprising SEQ ID NO:4 (MEDI0382), about 220 mM sorbitol, about 20 mM sodium phosphate, and about 0.4% w/v meta-cresol, and the pH of the pharmaceutical composition is about 8.1.

In certain embodiments, the composition comprises sodium hydroxide. In certain embodiments, the sodium phosphate comprises sodium phosphate monobasic monohydrate and sodium phosphate dibasic heptahydrate. In certain embodiments, the ratio of sodium phosphate monobasic monohydrate to sodium phosphate dibasic heptahydrate is about 0.5:19.5.

In certain embodiments, the composition comprises about 0.05 mg to about 0.5 mg of the peptide comprising SEQ ID NO:4 (MEDI0382). In certain embodiments, the composition comprises about 0.3 mg of the peptide of SEQ ID NO:4 (MEDI0382).

In certain embodiments, the composition is liquid. In certain embodiments, the composition is for parenteral administration. In certain embodiments, the composition is for subcutaneous administration.

In certain embodiments, a vial, a syringe, or a pen comprises a pharmaceutical composition provided herein. In certain embodiments, the vial, syringe, or pen is a multi-dose vial, syringe, or pen.

In certain embodiments a method of reducing body weight comprises administering to a human subject in need thereof a pharmaceutical composition provided herein.

In certain embodiments, a method of reducing body fat comprises administering to a human subject in need thereof a pharmaceutical composition provided herein.

In certain embodiments, a method of treating obesity comprises administering to a human subject in need thereof a pharmaceutical composition provided herein.

In certain embodiments, a method of treating or preventing a disease or condition caused or characterized by excess body weight comprises administering to a human subject in need thereof a pharmaceutical composition provided herein.

In certain embodiments, a method of treating Nonalcoholic Steatohepatitis (NASH) comprises administering to a human subject in need thereof a pharmaceutical composition provided herein. In certain embodiments, a method of treating Nonalcoholic Fatty Liver Disease (NAFLD) comprises administering to a human subject in need thereof a pharmaceutical composition provided herein.

In certain embodiments, a method of reducing liver fat comprises administering to a human subject in need thereof a pharmaceutical composition provided herein.

In certain embodiments, a method of increasing lipid oxidation comprises administering to a human subject in need thereof a pharmaceutical composition provided herein.

In certain embodiments, a method of reducing food intake comprising administering to a human subject in need thereof a pharmaceutical composition provided herein.

In certain embodiments, a method of lowering plasma glucose comprising administering to a human subject in need thereof a pharmaceutical composition provided herein.

In certain embodiments, of the methods provided herein the subject has diabetes. In certain embodiments, the diabetes is type 2 diabetes mellitus.

In certain embodiments, a method of treating type 2 diabetes mellitus comprises administering to a human in need thereof a pharmaceutical composition provided herein.

In certain embodiments, a method of improving glycemic control in a human subject with type 2 diabetes mellitus comprises administering to the subject a pharmaceutical composition provided herein.

In certain embodiments of the methods provided herein, the administration reduces body weight. In certain embodiments of the methods provided herein, the administration treats obesity. In certain embodiments of the methods provided herein, the administration reduces body fat.

In certain embodiments of the methods provided herein, about 0.05 mg to about 0.3 mg of the peptide is administered. In certain embodiments of the methods provided herein, about 0.05 mg, about 0.1 mg, about 0.15 mg, about 0.2 mg, about 0.25 mg, or about 0.3 mg of the peptide is administered.

In certain embodiments of the methods provided herein, the peptide is administered daily. In certain embodiments of the methods provided herein, the peptide is administered once daily. In certain embodiments of the methods provided herein, the peptide is administered for at least one week, for at least two weeks, for at least three weeks, or for at least four weeks.

In certain embodiments of the methods provided herein, the peptide is administered by injection. In certain embodiments of the methods provided herein, the administration is subcutaneous.

In certain embodiments of the methods provided herein, the subject has a body mass index (BMI) of 27 to 40 kg/m′. In certain embodiments of the methods provided herein, the subject has a BMI of 30-39.9 kg/m′. In certain embodiments of the methods provided herein, the subject has a BMI of at least 40 kg/m′. In certain embodiments of the methods provided herein, the subject is overweight. In certain embodiments of the methods provided herein, the subject is obese.

In certain embodiments of the methods provided herein, the administration is an adjunct to diet and exercise.

### BRIEF DESCRIPTION OF THE DRAWINGS

FIG. 1 shows the chemical structure, chemical formula (C₁₆₇H₂₅₂N₄₂O₅₅), and molecular weight (3728.09), for MEDI0382 (SEQ ID NO:4).

FIG. 2 shows the results of a Thioflavin T binding (ThT) assay in the presence of amino acids, citrate, and magnesium chloride. (See Example 11.)

FIG. 3 shows the results of a ThT assay in the presence trehalose, propylene glycol, sorbitol, sucrose, mannitol, lysine, and sodium citrate. (See Example 11.)

FIG. 4 shows the purity levels of different MEDI0382 compositions stored at 40° C. (See Example 11.)

FIG. 5 shows the effects of m-cresol and phenol on the purity and fibrillation of MEDI0382. (See Example 13.)

FIG. 6 shows the hydrodynamic radius (R(h)) and fibrillation (lag time) across ranges of pH, phenol, glycerol, and sorbitol. (See Example 13.)

FIG. 7 shows the purity of MEDI0382 in seven different compositions over 6 months at 25° C. (See Example 14.)

FIG. 8 shows the prediction profiler's estimate of the impact of the components in seven different MEDI0382 compositions on total purity (DS), impurities (oxidation, isomer 15, and isomer 9), and total impurities. (See Example 14.)

FIG. 9 shows the effect of liquid phenol on the chemical stability of 1 mg/mL MEDI0382 over 12 weeks at 5° C. and 25° C. (See Example 17.)

FIG. 10 shows the effect of solid phenol on the chemical stability of 1 mg/mL MEDI0382 over 12 weeks at 5° C. and 25° C. (See Example 17.)

FIG. 11 shows the effect of solid phenol on the chemical stability of 2 mg/mL MEDI0382 over 12 weeks at 5° C. and 25° C. (See Example 17.)

FIG. 12 shows the effect of m-cresol (Sigma) on the chemical stability of 1 mg/mL MEDI0382 over 12 weeks at 5° C. and 25° C. (See Example 17.)

FIG. 13 shows the effect of m-cresol (Hedinger) on the chemical stability of 1 mg/mL MEDI0382 over 12 weeks at 5° C. and 25° C. (See Example 17.)

FIGS. 14A, 14B, and 14C show the effect of sodium phosphate concentration and salt type on formation of high molecular weight (HWM) MEDI0382 impurities. (See Example 19.)

FIG. 15 shows the impact of buffer type on HMW impurities levels at 40° C. (SEC results). (See Example 19.)

FIG. 16A shows that 5 mg/ml formulations of MEDI0382 have lower levels of total impurities than 1 or 2 mg/ml formulations. (See Example 20.)

FIG. 16B shows the high molecular weight (HMW) impurities in 5 mg/ml and 1 mg/ml formulations of MEDI0382 at 40° C., 25° C., and 5° C. (See Example 20.)

FIG. 17 shows that there were no significant fibrillation positive particles identified by FACS in 1, 2, or 5 mg/ml formulations of MEDI0382. (See Example 20.)

FIG. 18 shows TEM images of fibrils in 1 and 5 mg/ml formulations of MEDI0382. (See Example 20.)

FIG. 19 shows total impurities in 5 mg/ml and 1 mg/ml formulations of MEDI0382 at 40° C., 25° C., and 5° C. (See Example 21.)

FIG. 20 shows high molecular weight (HMW) impurities in 5 mg/ml and 1 mg/ml formulations of MEDI0382 at 40° C., 25° C., and 5° C. (See Example 21.)

FIG. 21 shows ThT positive particles at 5° C. as observed by FACS. (See Example 21.)

FIG. 22 shows TEM images of fibrils in 1 and 5 mg/ml formulations of MEDI0382. (See Example 21.)

FIG. 23 shows the results of stability studies at 25° C. (left), 32° C. (middle), and 40° C. (right) on MEDI0382 compounded using oxygen displacement. DO=dissolved oxygen. The model lines refer to the Arrhenius model developed from stability study data using MEDI0382 compounded in normal atmospheric conditions. (See Example 23.)

FIG. 24 shows a comparison of stability data obtained using MEDI0382 compounded in 5% dissolved oxygen (DO) and 20% DO with an Arrhenius model of MEDI0382 compounded in normal atmospheric conditions. (See Example 23.)

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

### I. Definitions

As used in this specification, the singular forms “a,” “an” and “the” specifically also encompass the plural forms of the terms to which they refer, unless the content clearly dictates otherwise. As such, the terms “a” (or “an”), “one or more,” and “at least one” can be used interchangeably herein.

The term “about” is used herein to mean approximately, in the region of, roughly, or around. When the term “about” is used in conjunction with a numerical range, it modifies that range by extending the boundaries above and below the numerical values set forth. In general, unless otherwise stated, the term “about” is used herein to modify a numerical value above and below the stated value by a variance of 20%.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. A peptide “comprising” a particular amino acid sequence refers to a peptide containing the amino acid sequence, wherein the peptide may or may not contain additional amino acids or other modifications to the amino acid sequence. A peptide “consisting of” a particular amino acid sequence refers to a peptide containing only the amino acid sequence and no additional amino acids or other modifications to the amino acid sequence. A peptide “comprising” an amino acid sequence “consisting of” a particular amino acid sequence refers to a peptide containing the amino acid sequence and no additional amino acids; however, the peptide may comprise other modifications to the amino acid sequence (e.g., an acyl moiety or a palmitoyl moiety).

Technical and scientific terms used herein have the meaning commonly understood by one of ordinary skill in the art to which the present application pertains, unless otherwise defined. Reference is made herein to various methodologies and materials known to those of skill in the art. Standard reference works setting forth the general principles of peptide synthesis include W. C. Chan and P. D. White, “Fmoc Solid Phase Peptide Synthesis: A Practical Approach”, Oxford University Press, Oxford (2004). In addition, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The terms “peptide,” “polypeptide,” “protein,” and “protein fragment” are used interchangeably herein to refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The term “peptide” further includes peptides that have undergone post-translational or post-synthesis modifications, for example, glycosylation, acetylation, phosphorylation, amidation, derivatization by known protecting/blocking groups, proteolytic cleavage, or modification by non-naturally occurring amino acids. A “peptide” can be part of a fusion peptide comprising additional components such as, an Fc domain or an albumin domain, to increase half-life. A peptide as described herein can also be derivatized in a number of different ways.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

The term “isolated” refers to the state in which peptides or nucleic acids, will generally be in accordance with the present disclosure. Isolated peptides and isolated nucleic acids will be free or substantially free of material with which they are naturally associated such as other peptides or nucleic acids with which they are found in their natural environment, or the environment in which they are prepared (e.g. cell culture) when such preparation is by recombinant DNA technology practiced in vitro or in vivo. Peptides and nucleic acid can be formulated with diluents or adjuvants and still for practical purposes be isolated—for example the peptides will normally be mixed with gelatin or other carriers if used to coat microtitre plates for use in immunoassays, or will be mixed with pharmaceutically acceptable carriers or diluents when used in diagnosis or therapy.

A “recombinant” peptide refers to a peptide produced via recombinant DNA technology. Recombinantly produced peptides expressed in host cells are considered isolated for the purpose of the present disclosure, as are native or recombinant polypeptides which have been separated, fractionated, or partially or substantially purified by any suitable technique.

The terms “fragment,” “analog,” “derivative,” or “variant” when referring to a GLP-1/glucagon agonist peptide include any peptide which retains at least some desirable activity, e.g., binding to glucagon and/or GLP-1 receptors. Fragments of GLP-1/glucagon agonist peptides provided herein include proteolytic fragments, deletion fragments which exhibit desirable properties during expression, purification, and/or administration to a subject.

The term “variant,” as used herein, refers to a peptide that differs from the recited peptide due to amino acid substitutions, deletions, insertions, and/or modifications. Variants can be produced using art-known mutagenesis techniques. Variants can also, or alternatively, contain other modifications—for example a peptide can be conjugated or coupled, e.g., fused to a heterologous amino acid sequence or other moiety, e.g., for increasing half-life, solubility, or stability. Examples of moieties to be conjugated or coupled to a peptide provided herein include, but are not limited to, albumin, an immunoglobulin Fc region, polyethylene glycol (PEG), and the like. The peptide can also be conjugated or produced coupled to a linker or other sequence for ease of synthesis, purification or identification of the peptide (e.g., 6-His), or to enhance binding of the polypeptide to a solid support.

The terms “composition” or “pharmaceutical composition” refer to compositions containing a GLP-1/glucagon agonist peptide provided herein, along with e.g., pharmaceutically acceptable carriers, excipients, or diluents for administration to a subject in need of treatment, e.g., a human subject being treated for obesity.

The term “pharmaceutically acceptable” refers to compositions that are, within the scope of sound medical judgment, suitable for contact with the tissues of human beings and animals without excessive toxicity or other complications commensurate with a reasonable benefit/risk ratio.

The term “pharmaceutically acceptable carrier” refers to one or more non-toxic materials that do not interfere with the effectiveness of the biological activity of the GLP-1/glucagon agonist peptides.

An “effective amount” is that amount of a GLP-1/glucagon agonist peptide provided herein, the administration of which to a subject, either in a single dose or as part of a series, is effective for treatment, e.g., treatment of obesity. An amount is effective, for example, when its administration results in one or more of weight loss or weight maintenance (e.g., prevention of weight gain), loss of body fat, prevention or modulation of hypoglycemia, prevention or modulation hyperglycemia, promotion of insulin synthesis, or reduction in food intake. This amount can be a fixed dose for all subjects being treated, or can vary depending upon the weight, health, and physical condition of the subject to be treated, the extent of weight loss or weight maintenance desired, the formulation of peptide, a professional assessment of the medical situation, and other relevant factors.

The term “subject” is meant any subject, particularly a mammalian subject, in need of treatment with a GLP-1/glucagon agonist peptide provided herein. Mammalian subjects include, but are not limited to, humans, dogs, cats, guinea pigs, rabbits, rats, mice, horses, cattle, bears, cows, apes, monkeys, orangutans, and chimpanzees, and so on. In one embodiment, the subject is a human subject.

As used herein, a “subject in need thereof” refers to an individual for whom it is desirable to treat, e.g., to an obese subject or a subject prone to obesity for whom it is desirable to facilitate weight or body fat loss, weight or body fat maintenance, or to prevent or minimize weight gain over a specified period of time.

Terms such as “treating” or “treatment” or “to treat” refer to therapeutic measures that cure and/or halt progression of a diagnosed pathologic condition or disorder. Terms such as “preventing” refer to prophylactic or preventative measures that prevent and/or slow the development of a targeted pathologic condition or disorder. Thus, those in need of treatment include those already with the disease or condition. Those in need of prevention include those prone to have the disease or condition and those in whom the disease or condition is to be prevented. For example, the phrase “treating a patient” having a disease or condition caused or characterized by excess body weight refers to reducing the severity of the disease or condition to an extent that the subject no longer suffers discomfort and/or altered function due to it. The phrase “preventing” a disease or condition caused or characterized by excess body weight refers to reducing the potential for the disease or condition and/or reducing the occurrence of the disease or condition (for example a relative reduction in occurrence as compared to untreated patients).

Terms such as “decreasing the severity” refer to therapeutic measures that slow down or lessen the symptoms of a diagnosed pathologic condition or disorder. For example, the phrase “decreasing the severity” of a disease or condition caused or characterized by excess body weight refers to reducing the severity of the disease or condition (for example, a reduction in weight when compared to untreated patients or an increase in glucose control).

As used herein a “GLP-1/glucagon agonist peptide” is a chimeric peptide that exhibits activity at the glucagon receptor of at least about 1%, 5%, 10%, 20%, 30%, 40%, 50%, 60%, 70%, 80%, 90%, 95%, or more relative to native glucagon and also exhibits activity at the GLP-1 receptor of about at least about 1%, 5%, 10%, 20%, 30%, 40%, 50%, 60%, 70%, 80%, 90%, 95%, or more relative to native GLP-1, under the conditions of assay 1.

As used herein the term “native glucagon” refers to naturally-occurring glucagon, e.g., human glucagon, comprising the sequence of HSQGTFTSDYSKYLDSRRAQDFVQW LMNT (SEQ ID NO: 1). The term “native GLP-1” refers to naturally-occurring GLP-1, e.g., human GLP-1, and is a generic term that encompasses, e.g., GLP-1(7-36) amide (HAEGT FTSDVSSYLEGQAAKEFIAWLVKGR; SEQ ID NO: 2), GLP-1(7-37) acid (HAEGT FTSDVSSYLEGQAAKEFIAWLVKGRG; SEQ ID NO: 3), or a mixture of those two compounds. As used herein, a general reference to “glucagon” or “GLP-1” in the absence of any further designation is intended to mean native human glucagon or native human GLP-1, respectively. Unless otherwise indicated, “glucagon” refers to human glucagon, and “GLP-1” refers to human GLP-1.

### II. GLP-1/Glucagon Agonist Peptides

Provided herein are peptides which bind both to a glucagon receptor and to a GLP-1 receptor. Exemplary peptides such as MEDI0382 (G933; cotadutide) are provided in WO 2014/091316 and WO 2017/153575, each of which is herein incorporated by reference in its entirety. In certain embodiments, the peptide is MEDI0382, i.e., a 30 amino acid linear peptide with the sequence of HSQGTFTSDX₁₀SEYLDSERARDFVAWLEAGG-acid, wherein X₁₀=lysine with a palmitoyl group conjugated to the epsilon nitrogen, through a gamma glutamic acid linker (i.e., K(gE-palm)) (SEQ ID NO:4). In certain embodiments, the peptides provided herein are co-agonists of glucagon and GLP-1 activity. Such peptides are referred to herein as GLP-1/glucagon agonist peptides. GLP-1/glucagon agonist peptides as provided herein possess GLP-1 and glucagon activities with favorable ratios to promote weight loss, prevent weight gain, or to maintain a desirable body weight, and possess optimized solubility, formulatability, and stability. In certain embodiments, GLP-1/glucagon agonist peptides as provided herein are active at the human GLP1 and human glucagon receptors. In certain embodiments, GLP-1/glucagon agonist peptides as disclosed have desirable potencies at the glucagon and GLP-1 receptors, and have desirable relative potencies for promoting weight loss.

MEDI0382 has a glutamate residue at position 12, and maintains robust activity at both the glucagon and GLP-1 receptors. The corresponding residue is lysine in exendin-4 (exenatide) and glucagon and is serine in GLP-1. Although this residue is not thought to contact the receptor, changes in charge from positive to negative may modify the adjacent environment. Furthermore, MEDI0382 has a glutamate residue at position 27. Residue 27 is Lysine in exendin 4 and is an uncharged hydrophobic residue in GLP1 (valine) and glucagon (methionine). The lysine of exendin 4 makes electrostatic interactions with the GLP1 receptor at residues Glu127 and Glu24 (C. R. Underwood et al J Biol Chem 285 723-730 (2010); S. Runge et al J Biol Chem 283 11340-11347 (2008)). While a loss of GLP1R potency might be expected when the charge at position 27 is changed to negative, the change is compatible with GLP1R activity in MEDI0382.

MEDI0382 is palmitoylated to extend its half-life by association with serum albumin, thus reducing its propensity for renal clearance.

Alternatively or in addition, a GLP-1/glucagon agonist peptide as disclosed herein can be associated with a heterologous moiety, e.g., to extend half-life. The heterologous moiety can be a protein, a peptide, a protein domain, a linker, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, a cytokine, and a combination of two or more of such moieties.

### III. Methods of Making GLP-1/Glucagon Agonist Peptides

This disclosure provides a method of making a GLP-1/glucagon agonist peptide. GLP-1/glucagon agonist peptides provided herein can be made by any suitable method. For example, in certain embodiments the GLP-1/glucagon agonist peptides provided herein are chemically synthesized by methods well known to those of ordinary skill in the art, e.g., by solid phase synthesis as described by Merrifield (1963, J. Am. Chem. Soc. 85:2149-2154). Solid phase peptide synthesis can be accomplished, e.g., by using automated synthesizers, using standard reagents, e.g., as explained in Example 1 of WO 2014/091316.

Alternatively, GLP-1/glucagon agonist peptides provided herein can be produced recombinantly using a convenient vector/host cell combination as would be well known to the person of ordinary skill in the art. A variety of methods are available for recombinantly producing GLP-1/glucagon agonist peptides. Generally, a polynucleotide sequence encoding the GLP-1/glucagon agonist peptide is inserted into an appropriate expression vehicle, e.g., a vector which contains the necessary elements for the transcription and translation of the inserted coding sequence. The nucleic acid encoding the GLP-1/glucagon agonist peptide is inserted into the vector in proper reading frame. The expression vector is then transfected into a suitable host cell which will express the GLP-1/glucagon agonist peptide. Suitable host cells include without limitation bacteria, yeast, or mammalian cells. A variety of commercially-available host-expression vector systems can be utilized to express the GLP-1/glucagon agonist peptides described herein.

### IV. Pharmaceutical Compositions

Further provided are compositions, e.g., pharmaceutical compositions, that contain an effective amount of a GLP-1/glucagon agonist peptide (e.g., MEDI0382) as provided herein, formulated for the treatment of metabolic diseases, e.g., obesity.

In certain embodiments, a pharmaceutical composition comprising a GLP-1/glucagon agonist peptide (e.g., MEDI0382) is a liquid. In certain embodiments, a pharmaceutical composition comprising a GLP-1/glucagon agonist peptide (e.g., MEDI0382) is formulated for parenteral administration. In certain embodiments, a pharmaceutical composition comprising a GLP-1/glucagon agonist peptide (e.g., MEDI0382) is a liquid formulated for parenteral administration.

In certain embodiments, a pharmaceutical composition comprising a GLP-1/glucagon agonist peptide (e.g., MEDI0382) contains at least one fixed dose. In certain embodiments, a pharmaceutical composition comprising a GLP-1/glucagon agonist peptide (e.g., MEDI0382) contains one to ten fixed doses. In certain embodiments, a pharmaceutical composition comprising a GLP-1/glucagon agonist peptide (e.g., MEDI0382) contains one to six fixed doses (e.g., 50, 100, 150, 200, 250, 300, 350, 400, 450, 500, 550, and 600 mcg).

In certain embodiments, a pharmaceutical composition comprising a GLP-1/glucagon agonist peptide (e.g., MEDI0382) has a shelf-life of at least 12 months at refrigerated conditions (2-8° C.). In certain embodiments, a pharmaceutical composition comprising a GLP-1/glucagon agonist peptide (e.g., MEDI0382) has a shelf-life of at least 2 years at refrigerated conditions (2-8° C.).

In certain embodiments, a pharmaceutical composition comprises about 0.5 to about 5 mg/ml of a GLP-1/glucagon agonist peptide (e.g., MEDI0382). In certain embodiments, a pharmaceutical composition comprises about 1 mg/ml of a GLP-1/glucagon agonist peptide (e.g., MEDI0382). In certain embodiments, a pharmaceutical composition comprises about 2 mg/ml of a GLP-1/glucagon agonist peptide (e.g., MEDI0382). In certain embodiments, a pharmaceutical composition comprises about 5 mg/ml of a GLP-1/glucagon agonist peptide (e.g., MEDI0382).

In certain embodiments, a pharmaceutical composition comprises about 0.05 mg to about 0.5 mg of a GLP-1/glucagon agonist peptide (e.g., MEDI0382). In certain embodiments, a pharmaceutical composition comprises about 0.3 mg of a GLP-1/glucagon agonist peptide (e.g., MEDI0382).

In certain embodiments, a pharmaceutical composition comprising a GLP-1/glucagon agonist peptide (e.g., MEDI0382) has a pH of at least 7.9. In certain embodiments, a pharmaceutical composition comprising a GLP-1/glucagon agonist peptide (e.g., MEDI0382) has a pH of about 7.9 to about 8.5. In certain embodiments, a pharmaceutical composition comprising a GLP-1/glucagon agonist peptide (e.g., MEDI0382) has a pH of about 7.9 to about 8.4. In certain embodiments, a pharmaceutical composition comprising a GLP-1/glucagon agonist peptide (e.g., MEDI0382) has a pH of about 7.9 to about 8.3. In certain embodiments, a pharmaceutical composition comprising a GLP-1/glucagon agonist peptide (e.g., MEDI0382) has a pH of about 7.9 to about 8.2. In certain embodiments, a pharmaceutical composition comprising a GLP-1/glucagon agonist peptide (e.g., MEDI0382) has a pH of about 7.9 to about 8.1.

In certain embodiments, a pharmaceutical composition comprising a GLP-1/glucagon agonist peptide (e.g., MEDI0382) has a pH of at least 8. In certain embodiments, a pharmaceutical composition comprising a GLP-1/glucagon agonist peptide (e.g., MEDI0382) has a pH of about 8 to about 8.5. In certain embodiments, a pharmaceutical composition comprising a GLP-1/glucagon agonist peptide (e.g., MEDI0382) has a pH of about 8 to about 8.4. In certain embodiments, a pharmaceutical composition comprising a GLP-1/glucagon agonist peptide (e.g., MEDI0382) has a pH of about 8 to about 8.3. In certain embodiments, a pharmaceutical composition comprising a GLP-1/glucagon agonist peptide (e.g., MEDI0382) has a pH of about 8 to about 8.2. In certain embodiments, a pharmaceutical composition comprising a GLP-1/glucagon agonist peptide (e.g., MEDI0382) has a pH of about 8.1 to about 8.5. In certain embodiments, a pharmaceutical composition comprising a GLP-1/glucagon agonist peptide (e.g., MEDI0382) has a pH of about 8.1 to about 8.4. In certain embodiments, a pharmaceutical composition comprising a GLP-1/glucagon agonist peptide (e.g., MEDI0382) has a pH of about 8.1 to about 8.3. In certain embodiments, a pharmaceutical composition comprising a GLP-1/glucagon agonist peptide (e.g., MEDI0382) has a pH of about 8.1 to about 8.2. In certain embodiments, a pharmaceutical composition comprising a GLP-1/glucagon agonist peptide (e.g., MEDI0382) has a pH of about 8.1. In certain embodiments, a pharmaceutical composition comprising a GLP-1/glucagon agonist peptide (e.g., MEDI0382) has a pH of about 8.2. In certain embodiments, a pharmaceutical composition comprising a GLP-1/glucagon agonist peptide (e.g., MEDI0382) has a pH of about 8.4.

In certain embodiments, a pharmaceutical composition comprising a GLP-1/glucagon agonist peptide (e.g., MEDI0382) comprises a pH-adjusting agent. In some embodiments, the pH-adjusting agent is sodium hydroxide. In some embodiments, a pharmaceutical composition comprising a GLP-1/glucagon agonist peptide (e.g., MEDI0382) comprises sodium hydroxide.

In certain embodiments, a pharmaceutical composition comprising a GLP-1/glucagon agonist peptide (e.g., MEDI0382) comprises sodium hydroxide at a concentration sufficient to make the pH of the composition at least 7.9. In certain embodiments, a pharmaceutical composition comprising a GLP-1/glucagon agonist peptide (e.g., MEDI0382) comprises sodium hydroxide at a concentration sufficient to make the pH of the composition about 7.9 to about 8.5. In certain embodiments, a pharmaceutical composition comprising a GLP-1/glucagon agonist peptide (e.g., MEDI0382) comprises sodium hydroxide at a concentration sufficient to make the pH of the composition about 7.9 to about 8.4. In certain embodiments, a pharmaceutical composition comprising a GLP-1/glucagon agonist peptide (e.g., MEDI0382) comprises sodium hydroxide at a concentration sufficient to make the pH of the composition about 7.9 to about 8.3. In certain embodiments, a pharmaceutical composition comprising a GLP-1/glucagon agonist peptide (e.g., MEDI0382) comprises sodium hydroxide at a concentration sufficient to make the pH of the composition about 7.9 to about 8.2. In certain embodiments, a pharmaceutical composition comprising a GLP-1/glucagon agonist peptide (e.g., MEDI0382) comprises sodium hydroxide at a concentration sufficient to make the pH of the composition about 7.9 to about 8.1.

In certain embodiments, a pharmaceutical composition comprising a GLP-1/glucagon agonist peptide (e.g., MEDI0382) comprises sodium hydroxide at a concentration sufficient to make the pH of the composition at least 8. In certain embodiments, a pharmaceutical composition comprising a GLP-1/glucagon agonist peptide (e.g., MEDI0382) comprises sodium hydroxide at a concentration sufficient to make the pH of the composition about 8 to about 8.5. In certain embodiments, a pharmaceutical composition comprising a GLP-1/glucagon agonist peptide (e.g., MEDI0382) comprises sodium hydroxide at a concentration sufficient to make the pH of the composition about 8 to about 8.4. In certain embodiments, a pharmaceutical composition comprising a GLP-1/glucagon agonist peptide (e.g., MEDI0382) comprises sodium hydroxide at a concentration sufficient to make the pH of the composition about 8 to about 8.3. In certain embodiments, a pharmaceutical composition comprising a GLP-1/glucagon agonist peptide (e.g., MEDI0382) comprises sodium hydroxide at a concentration sufficient to make the pH of the composition about 8 to about 8.2. In certain embodiments, a pharmaceutical composition comprising a GLP-1/glucagon agonist peptide (e.g., MEDI0382) comprises sodium hydroxide at a concentration sufficient to make the pH of the composition about 8.1 to about 8.5. In certain embodiments, a pharmaceutical composition comprising a GLP-1/glucagon agonist peptide (e.g., MEDI0382) has a pH comprises sodium hydroxide at a concentration sufficient to make the pH of the composition about 8.1 to about 8.4. In certain embodiments, a pharmaceutical composition comprising a GLP-1/glucagon agonist peptide (e.g., MEDI0382) comprises sodium hydroxide at a concentration sufficient to make the pH of the composition about 8.1 to about 8.3. In certain embodiments, a pharmaceutical composition comprising a GLP-1/glucagon agonist peptide (e.g., MEDI0382) comprises sodium hydroxide at a concentration sufficient to make the pH of the composition about 8.1 to about 8.2. In certain embodiments, a pharmaceutical composition comprising a GLP-1/glucagon agonist peptide (e.g., MEDI0382) comprises sodium hydroxide at a concentration sufficient to make the pH of the composition about 8.1.

In certain embodiments, a pharmaceutical composition comprising a GLP-1/glucagon agonist peptide (e.g., MEDI0382) comprises a tonicity agent. In certain embodiments, the tonicity agent is sorbitol, mannitol, or propylene glycol.

In certain embodiments, a pharmaceutical composition comprising a GLP-1/glucagon agonist peptide (e.g., MEDI0382) comprises sorbitol. In certain embodiments, a pharmaceutical composition comprising a GLP-1/glucagon agonist peptide (e.g., MEDI0382) comprises about 190 mM to about 270 mM sorbitol. In certain embodiments, a pharmaceutical composition comprising a GLP-1/glucagon agonist peptide (e.g., MEDI0382) comprises about 190 mM to about 250 mM sorbitol. In certain embodiments, a pharmaceutical composition comprising a GLP-1/glucagon agonist peptide (e.g., MEDI0382) comprises about 200 mM to about 250 mM sorbitol. In certain embodiments, a pharmaceutical composition comprising a GLP-1/glucagon agonist peptide (e.g., MEDI0382) comprises about 210 mM to about 250 mM sorbitol. In certain embodiments, a pharmaceutical composition comprising a GLP-1/glucagon agonist peptide (e.g., MEDI0382) comprises about 220 mM to about 250 mM sorbitol. In certain embodiments, a pharmaceutical composition comprising a GLP-1/glucagon agonist peptide (e.g., MEDI0382) comprises about 200 mM to about 240 mM sorbitol. In certain embodiments, a pharmaceutical composition comprising a GLP-1/glucagon agonist peptide (e.g., MEDI0382) comprises about 210 mM to about 240 mM sorbitol. In certain embodiments, a pharmaceutical composition comprising a GLP-1/glucagon agonist peptide (e.g., MEDI0382) comprises about 220 mM to about 240 mM sorbitol. In certain embodiments, a pharmaceutical composition comprising a GLP-1/glucagon agonist peptide (e.g., MEDI0382) comprises about 210 mM to about 230 mM sorbitol. In certain embodiments, a pharmaceutical composition comprising a GLP-1/glucagon agonist peptide (e.g., MEDI0382) comprises about 220 mM to about 230 mM sorbitol. In certain embodiments, a pharmaceutical composition comprising a GLP-1/glucagon agonist peptide (e.g., MEDI0382) comprises about 200 mM to about 220 mM sorbitol. In certain embodiments, a pharmaceutical composition comprising a GLP-1/glucagon agonist peptide (e.g., MEDI0382) comprises about 210 mM to about 220 mM sorbitol. In certain embodiments, a pharmaceutical composition comprising a GLP-1/glucagon agonist peptide (e.g., MEDI0382) comprises about 215 mM to about 225 mM sorbitol. In certain embodiments, a pharmaceutical composition comprising a GLP-1/glucagon agonist peptide (e.g., MEDI0382) comprises about 219 mM to about 221 mM sorbitol. In certain embodiments, a pharmaceutical composition comprising a GLP-1/glucagon agonist peptide (e.g., MEDI0382) comprises about 220 mM sorbitol. In certain embodiments, a pharmaceutical composition comprising a GLP-1/glucagon agonist peptide (e.g., MEDI0382) comprises 220.1, 220.2, 220.3, 220.4, or 220.5 mM sorbitol. In certain embodiments, a pharmaceutical composition comprising a GLP-1/glucagon agonist peptide (e.g., MEDI0382) comprises 220.3 mM sorbitol.

In certain embodiments, a pharmaceutical composition comprising a GLP-1/glucagon agonist peptide (e.g., MEDI0382) comprises about 35 mg/mL to about 45 mg/mL sorbitol. In certain embodiments, a pharmaceutical composition comprising a GLP-1/glucagon agonist peptide (e.g., MEDI0382) comprises about 40 mg/mL to about 41 mg/mL sorbitol. In certain embodiments, a pharmaceutical composition comprising a GLP-1/glucagon agonist peptide (e.g., MEDI0382) comprises about 40 mg/mL to about 40.5 mg/mL sorbitol. In certain embodiments, a pharmaceutical composition comprising a GLP-1/glucagon agonist peptide (e.g., MEDI0382) comprises about 40.1 mg/mL to about 40.2 mg/mL sorbitol. In certain embodiments, a pharmaceutical composition comprising a GLP-1/glucagon agonist peptide (e.g., MEDI0382) comprises about 40.13 mg/mL sorbitol.

In certain embodiments, a pharmaceutical composition comprising a GLP-1/glucagon agonist peptide (e.g., MEDI0382) comprises mannitol. In certain embodiments, a pharmaceutical composition comprising a GLP-1/glucagon agonist peptide (e.g., MEDI0382) comprises about 50 mM to about 300 mM mannitol. In certain embodiments, a pharmaceutical composition comprising a GLP-1/glucagon agonist peptide (e.g., MEDI0382) comprises about 100 mM to about 300 mM mannitol. In certain embodiments, a pharmaceutical composition comprising a GLP-1/glucagon agonist peptide (e.g., MEDI0382) comprises about 150 mM to about 300 mM mannitol. In certain embodiments, a pharmaceutical composition comprising a GLP-1/glucagon agonist peptide (e.g., MEDI0382) comprises about 50 mM mannitol. In certain embodiments, a pharmaceutical composition comprising a GLP-1/glucagon agonist peptide (e.g., MEDI0382) comprises about 100 mM mannitol. In certain embodiments, a pharmaceutical composition comprising a GLP-1/glucagon agonist peptide (e.g., MEDI0382) comprises about 150 mM mannitol. In certain embodiments, a pharmaceutical composition comprising a GLP-1/glucagon agonist peptide (e.g., MEDI0382) comprises about 200 mM mannitol. In certain embodiments, a pharmaceutical composition comprising a GLP-1/glucagon agonist peptide (e.g., MEDI0382) comprises about 220 mM mannitol. In certain embodiments, a pharmaceutical composition comprising a GLP-1/glucagon agonist peptide (e.g., MEDI0382) comprises about 250 mM mannitol. In certain embodiments, a pharmaceutical composition comprising a GLP-1/glucagon agonist peptide (e.g., MEDI0382) comprises about 300 mM mannitol.

In certain embodiments, a pharmaceutical composition comprising a GLP-1/glucagon agonist peptide (e.g., MEDI0382) comprises propylene glycol. In certain embodiments, a pharmaceutical composition comprising a GLP-1/glucagon agonist peptide (e.g., MEDI0382) comprises about 0.05% (w/v) to about 2% (w/v) propylene glycol. In certain embodiments, a pharmaceutical composition comprising a GLP-1/glucagon agonist peptide (e.g., MEDI0382) comprises about 1% (w/v) to about 2% (w/v) propylene glycol. In certain embodiments, a pharmaceutical composition comprising a GLP-1/glucagon agonist peptide (e.g., MEDI0382) comprises about 1.5% (w/v) to about 2% (w/v) propylene glycol. In certain embodiments, a pharmaceutical composition comprising a GLP-1/glucagon agonist peptide (e.g., MEDI0382) comprises about 1% (w/v) propylene glycol. In certain embodiments, a pharmaceutical composition comprising a GLP-1/glucagon agonist peptide (e.g., MEDI0382) comprises about 1.35% (w/v) propylene glycol. In certain embodiments, a pharmaceutical composition comprising a GLP-1/glucagon agonist peptide (e.g., MEDI0382) comprises about 1.5% (w/v) propylene glycol. In certain embodiments, a pharmaceutical composition comprising a GLP-1/glucagon agonist peptide (e.g., MEDI0382) comprises about 1.85% (w/v) propylene glycol. In certain embodiments, a pharmaceutical composition comprising a GLP-1/glucagon agonist peptide (e.g., MEDI0382) comprises about 2% (w/v) propylene glycol.

In certain embodiments, a pharmaceutical composition comprising a GLP-1/glucagon agonist peptide (e.g., MEDI0382) comprises a preservative/anti-microbial agent. In certain embodiments, the preservative or anti-microbial agent is meta-cresol (m-cresol) or phenol.

In certain embodiments, a pharmaceutical composition comprising a GLP-1/glucagon agonist peptide (e.g., MEDI0382) comprises meta-cresol (m-cresol). In certain embodiments, a pharmaceutical composition comprising a GLP-1/glucagon agonist peptide (e.g., MEDI0382) comprises about 0.2% (w/v) to about 0.5% (w/v) m-cresol. In certain embodiments, a pharmaceutical composition comprising a GLP-1/glucagon agonist peptide (e.g., MEDI0382) comprises at least 0.27% (w/v) m-cresol. In certain embodiments, a pharmaceutical composition comprising a GLP-1/glucagon agonist peptide (e.g., MEDI0382) comprises about 0.27% (w/v) to about 0.45% (w/v) m-cresol. In certain embodiments, a pharmaceutical composition comprising a GLP-1/glucagon agonist peptide (e.g., MEDI0382) comprises about 0.27% (w/v) to about 0.4% (w/v) m-cresol. In certain embodiments, a pharmaceutical composition comprising a GLP-1/glucagon agonist peptide (e.g., MEDI0382) comprises about 0.27% (w/v) to about 0.35% (w/v) m-cresol. In certain embodiments, a pharmaceutical composition comprising a GLP-1/glucagon agonist peptide (e.g., MEDI0382) comprises about 0.28% (w/v) to about 0.34% (w/v) m-cresol. In certain embodiments, a pharmaceutical composition comprising a GLP-1/glucagon agonist peptide (e.g., MEDI0382) comprises about 0.29% (w/v) to about 0.33% (w/v) m-cresol. In certain embodiments, a pharmaceutical composition comprising a GLP-1/glucagon agonist peptide (e.g., MEDI0382) comprises about 0.3% (w/v) to about 0.32% (w/v) m-cresol. In certain embodiments, a pharmaceutical composition comprising a GLP-1/glucagon agonist peptide (e.g., MEDI0382) comprises about 0.31% w/v m-cresol.

In certain embodiments, a pharmaceutical composition comprising a GLP-1/glucagon agonist peptide (e.g., MEDI0382) comprises at least 0.34% (w/v) m-cresol. In certain embodiments, a pharmaceutical composition comprising a GLP-1/glucagon agonist peptide (e.g., MEDI0382) comprises about 0.34% (w/v) to about 0.45% w/v m-cresol. In certain embodiments, a pharmaceutical composition comprising a GLP-1/glucagon agonist peptide (e.g., MEDI0382) comprises about 0.38% (w/v) to about 0.42% w/v m-cresol. In certain embodiments, a pharmaceutical composition comprising a GLP-1/glucagon agonist peptide (e.g., MEDI0382) comprises about 0.39% (w/v) to about 0.41% w/v m-cresol. In certain embodiments, a pharmaceutical composition comprising a GLP-1/glucagon agonist peptide (e.g., MEDI0382) comprises about 0.4% w/v m-cresol.

In certain embodiments, a pharmaceutical composition comprising a GLP-1/glucagon agonist peptide (e.g., MEDI0382) comprises about 2.7 mg/ml to about 4.5 mg/ml m-cresol. In certain embodiments, a pharmaceutical composition comprising a GLP-1/glucagon agonist peptide (e.g., MEDI0382) comprises about 2 mg/ml to about 4 mg/ml m-cresol. In certain embodiments, a pharmaceutical composition comprising a GLP-1/glucagon agonist peptide (e.g., MEDI0382) comprises about 3 mg/ml to about 3.5 mg/ml m-cresol. In certain embodiments, a pharmaceutical composition comprising a GLP-1/glucagon agonist peptide (e.g., MEDI0382) comprises about 3.1 mg/ml m-cresol.

In certain embodiments, a pharmaceutical composition comprising a GLP-1/glucagon agonist peptide (e.g., MEDI0382) comprises about 3 mg/ml to about 5 mg/ml m-cresol. In certain embodiments, a pharmaceutical composition comprising a GLP-1/glucagon agonist peptide (e.g., MEDI0382) comprises about 3.5 mg/ml to about 4.5 mg/ml m-cresol. In certain embodiments, a pharmaceutical composition comprising a GLP-1/glucagon agonist peptide (e.g., MEDI0382) comprises about 4 mg/ml m-cresol.

In certain embodiments, a pharmaceutical composition comprising a GLP-1/glucagon agonist peptide (e.g., MEDI0382) comprises phenol. In certain embodiments, a pharmaceutical composition comprising a GLP-1/glucagon agonist peptide (e.g., MEDI0382) comprises about 0.05% (w/v) to about 2% (w/v) phenol. In certain embodiments, a pharmaceutical composition comprising a GLP-1/glucagon agonist peptide (e.g., MEDI0382) comprises about 0.1% (w/v) to about 2% (w/v) phenol. In certain embodiments, a pharmaceutical composition comprising a GLP-1/glucagon agonist peptide (e.g., MEDI0382) comprises about 0.2% (w/v) to about 2% (w/v) phenol. In certain embodiments, a pharmaceutical composition comprising a GLP-1/glucagon agonist peptide (e.g., MEDI0382) comprises about 0.3% (w/v) to about 2% (w/v) phenol. In certain embodiments, a pharmaceutical composition comprising a GLP-1/glucagon agonist peptide (e.g., MEDI0382) comprises about 0.4% (w/v) to about 2% (w/v) phenol. In certain embodiments, a pharmaceutical composition comprising a GLP-1/glucagon agonist peptide (e.g., MEDI0382) comprises about 0.05% (w/v) to about 1% (w/v) phenol. In certain embodiments, a pharmaceutical composition comprising a GLP-1/glucagon agonist peptide (e.g., MEDI0382) comprises about 0.1% (w/v) to about 1% (w/v) phenol. In certain embodiments, a pharmaceutical composition comprising a GLP-1/glucagon agonist peptide (e.g., MEDI0382) comprises about 0.2% (w/v) to about 1% (w/v) phenol. In certain embodiments, a pharmaceutical composition comprising a GLP-1/glucagon agonist peptide (e.g., MEDI0382) comprises about 0.3% (w/v) to about 1% (w/v) phenol. In certain embodiments, a pharmaceutical composition comprising a GLP-1/glucagon agonist peptide (e.g., MEDI0382) comprises about 0.4% (w/v) to about 1% (w/v) phenol. In certain embodiments, a pharmaceutical composition comprising a GLP-1/glucagon agonist peptide (e.g., MEDI0382) comprises about 0.4% (w/v) to about 0.6% (w/v) phenol. In certain embodiments, a pharmaceutical composition comprising a GLP-1/glucagon agonist peptide (e.g., MEDI0382) comprises about 0.4% (w/v) to about 0.5% (w/v) phenol. In certain embodiments, a pharmaceutical composition comprising a GLP-1/glucagon agonist peptide (e.g., MEDI0382) comprises about 0.5% (w/v) to about 0.6% (w/v) phenol.

In certain embodiments, a pharmaceutical composition comprising a GLP-1/glucagon agonist peptide (e.g., MEDI0382) comprises about 0.35% (w/v) phenol. In certain embodiments, a pharmaceutical composition comprising a GLP-1/glucagon agonist peptide (e.g., MEDI0382) comprises about 0.4% (w/v) phenol. In certain embodiments, a pharmaceutical composition comprising a GLP-1/glucagon agonist peptide (e.g., MEDI0382) comprises about 0.45% (w/v) phenol. In certain embodiments, a pharmaceutical composition comprising a GLP-1/glucagon agonist peptide (e.g., MEDI0382) comprises about 0.5% (w/v) phenol. In certain embodiments, a pharmaceutical composition comprising a GLP-1/glucagon agonist peptide (e.g., MEDI0382) comprises about 0.5% (w/v) phenol. In certain embodiments, a pharmaceutical composition comprising a GLP-1/glucagon agonist peptide (e.g., MEDI0382) comprises about 0.55% (w/v) phenol. In certain embodiments, a pharmaceutical composition comprising a GLP-1/glucagon agonist peptide (e.g., MEDI0382) comprises about 0.56% (w/v) phenol.

In certain embodiments, a pharmaceutical composition comprising a GLP-1/glucagon agonist peptide (e.g., MEDI0382) comprises a buffer. In certain embodiments, the buffer is sodium phosphate or TRIS.

In certain embodiments, a pharmaceutical composition comprising a GLP-1/glucagon agonist peptide (e.g., MEDI0382) comprises sodium phosphate.

In certain embodiments, a pharmaceutical composition comprising a GLP-1/glucagon agonist peptide (e.g., MEDI0382) comprises up to 30 mM sodium phosphate. In certain embodiments, a pharmaceutical composition comprising a GLP-1/glucagon agonist peptide (e.g., MEDI0382) comprises about 5 mM to about 30 mM sodium phosphate. In certain embodiments, a pharmaceutical composition comprising a GLP-1/glucagon agonist peptide (e.g., MEDI0382) comprises about 10 mM to about 30 mM sodium phosphate. In certain embodiments, a pharmaceutical composition comprising a GLP-1/glucagon agonist peptide (e.g., MEDI0382) comprises about 5 mM to about 25 mM sodium phosphate. In certain embodiments, a pharmaceutical composition comprising a GLP-1/glucagon agonist peptide (e.g., MEDI0382) comprises about 10 mM to about 25 mM sodium phosphate. In certain embodiments, a pharmaceutical composition comprising a GLP-1/glucagon agonist peptide (e.g., MEDI0382) comprises about 15 mM to about 25 mM sodium phosphate. In certain embodiments, a pharmaceutical composition comprising a GLP-1/glucagon agonist peptide (e.g., MEDI0382) comprises about 18 mM to about 22 mM sodium phosphate. In certain embodiments, a pharmaceutical composition comprising a GLP-1/glucagon agonist peptide (e.g., MEDI0382) comprises about 5 mM to about 20 mM sodium phosphate. In certain embodiments, a pharmaceutical composition comprising a GLP-1/glucagon agonist peptide (e.g., MEDI0382) comprises about 10 mM to about 20 mM sodium phosphate. In certain embodiments, a pharmaceutical composition comprising a GLP-1/glucagon agonist peptide (e.g., MEDI0382) comprises about 15 mM to about 20 mM sodium phosphate. In certain embodiments, a pharmaceutical composition comprising a GLP-1/glucagon agonist peptide (e.g., MEDI0382) comprises about 10 mM sodium phosphate. In certain embodiments, a pharmaceutical composition comprising a GLP-1/glucagon agonist peptide (e.g., MEDI0382) comprises about 20 mM sodium phosphate. In certain embodiments, a pharmaceutical composition comprising a GLP-1/glucagon agonist peptide (e.g., MEDI0382) comprises about 20.1 mM sodium phosphate. In certain embodiments, a pharmaceutical composition comprising a GLP-1/glucagon agonist peptide (e.g., MEDI0382) comprises about 50 mM sodium phosphate.

In certain embodiments, a pharmaceutical composition comprising a GLP-1/glucagon agonist peptide (e.g., MEDI0382) comprises about 1 to about 10 mg/mL sodium phosphate. In certain embodiments, a pharmaceutical composition comprising a GLP-1/glucagon agonist peptide (e.g., MEDI0382) comprises about 1 to about 9 mg/mL sodium phosphate. In certain embodiments, a pharmaceutical composition comprising a GLP-1/glucagon agonist peptide (e.g., MEDI0382) comprises about 1 to about 8 mg/mL sodium phosphate. In certain embodiments, a pharmaceutical composition comprising a GLP-1/glucagon agonist peptide (e.g., MEDI0382) comprises about 1 to about 7 mg/mL sodium phosphate. In certain embodiments, a pharmaceutical composition comprising a GLP-1/glucagon agonist peptide (e.g., MEDI0382) comprises about 1 to about 6 mg/mL sodium phosphate. In certain embodiments, a pharmaceutical composition comprising a GLP-1/glucagon agonist peptide (e.g., MEDI0382) comprises about 2 to about 10 mg/mL sodium phosphate. In certain embodiments, a pharmaceutical composition comprising a GLP-1/glucagon agonist peptide (e.g., MEDI0382) comprises about 2 to about 8 mg/mL sodium phosphate. In certain embodiments, a pharmaceutical composition comprising a GLP-1/glucagon agonist peptide (e.g., MEDI0382) comprises about 2 to about 6 mg/mL sodium phosphate. In certain embodiments, a pharmaceutical composition comprising a GLP-1/glucagon agonist peptide (e.g., MEDI0382) comprises about 3 to about 10 mg/mL sodium phosphate. In certain embodiments, a pharmaceutical composition comprising a GLP-1/glucagon agonist peptide (e.g., MEDI0382) comprises about 3 to about 8 mg/mL sodium phosphate. In certain embodiments, a pharmaceutical composition comprising a GLP-1/glucagon agonist peptide (e.g., MEDI0382) comprises about 3 to about 6 mg/mL sodium phosphate. In certain embodiments, a pharmaceutical composition comprising a GLP-1/glucagon agonist peptide (e.g., MEDI0382) comprises about 4 to about 10 mg/mL sodium phosphate. In certain embodiments, a pharmaceutical composition comprising a GLP-1/glucagon agonist peptide (e.g., MEDI0382) comprises about 4 to about 8 mg/mL sodium phosphate. In certain embodiments, a pharmaceutical composition comprising a GLP-1/glucagon agonist peptide (e.g., MEDI0382) comprises about 4 to about 6 mg/mL sodium phosphate. In certain embodiments, a pharmaceutical composition comprising a GLP-1/glucagon agonist peptide (e.g., MEDI0382) comprises about 4 to about 10 mg/mL sodium phosphate. In certain embodiments, a pharmaceutical composition comprising a GLP-1/glucagon agonist peptide (e.g., MEDI0382) comprises about 4 to about 8 mg/mL sodium phosphate. In certain embodiments, a pharmaceutical composition comprising a GLP-1/glucagon agonist peptide (e.g., MEDI0382) comprises about 5 to about 6 mg/mL sodium phosphate. In certain embodiments, a pharmaceutical composition comprising a GLP-1/glucagon agonist peptide (e.g., MEDI0382) comprises about 5.25 mg/mL sodium phosphate.

In certain embodiments, the sodium phosphate comprises sodium phosphate monobasic monohydrate. In certain embodiments, the sodium phosphate comprises sodium phosphate dibasic heptahydrate. In certain embodiments, the sodium phosphate comprises sodium phosphate monobasic monohydrate and sodium phosphate dibasic heptahydrate. In certain embodiments, the sodium phosphate comprises about 0.13 mg/mL sodium phosphate monobasic monohydrate and about 5.12 mg/mL sodium phosphate dibasic heptahydrate. In certain embodiments, the ratio of sodium phosphate monobasic monohydrate to sodium phosphate dibasic heptahydrate is about 0.25:19.5 to about 1:19.5. In certain embodiments, the ratio of sodium phosphate monobasic monohydrate to sodium phosphate dibasic heptahydrate is about 0.5:19.5.

In certain embodiments, 20 mM sodium phosphate comprises about 0.5 mM sodium phosphate monobasic monohydrate and about 19.5 mM sodium phosphate dibasic heptahydrate. In certain embodiments, 20 mM sodium phosphate comprises 1 mM sodium phosphate monobasic monohydrate and about 19 mM sodium phosphate dibasic heptahydrate. In certain embodiments, 20.1 mM sodium phosphate comprises about 1 mM sodium phosphate monobasic monohydrate and about 19.1 mM sodium phosphate dibasic heptahydrate.

In certain embodiments, a pharmaceutical composition comprising a GLP-1/glucagon agonist peptide (e.g., MEDI0382) comprises TRIS. In certain embodiments, a pharmaceutical composition comprising a GLP-1/glucagon agonist peptide (e.g., MEDI0382) comprises about 25 mM to about 150 mM TRIS (e.g., pH 7.5). In certain embodiments, a pharmaceutical composition comprising a GLP-1/glucagon agonist peptide (e.g., MEDI0382) comprises about 50 mM to about 100 mM TRIS (e.g., pH 7.5). In certain embodiments, a pharmaceutical composition comprising a GLP-1/glucagon agonist peptide (e.g., MEDI0382) comprises about 25 mM TRIS (e.g., pH 7.5). In certain embodiments, a pharmaceutical composition comprising a GLP-1/glucagon agonist peptide (e.g., MEDI0382) comprises about 50 mM TRIS (e.g., pH 7.5). In certain embodiments, a pharmaceutical composition comprising a GLP-1/glucagon agonist peptide (e.g., MEDI0382) comprises about 75 mM TRIS (e.g., pH 7.5). In certain embodiments, a pharmaceutical composition comprising a GLP-1/glucagon agonist peptide (e.g., MEDI0382) comprises about 100 mM TRIS (e.g., pH 7.5). In certain embodiments, a pharmaceutical composition comprising a GLP-1/glucagon agonist peptide (e.g., MEDI0382) comprises about 125 mM TRIS (e.g., pH 7.5). In certain embodiments, a pharmaceutical composition comprising a GLP-1/glucagon agonist peptide (e.g., MEDI0382) comprises about 150 mM TRIS (e.g., pH 7.5).

In certain embodiments, a pharmaceutical composition comprising a GLP-1/glucagon agonist peptide (e.g., MEDI0382) does not contain sodium phosphate. In certain embodiments, a pharmaceutical composition comprising a GLP-1/glucagon agonist peptide (e.g., MEDI0382) does not contain a buffer.

In certain embodiments, a pharmaceutical composition comprising a GLP-1/glucagon agonist peptide (e.g., MEDI0382) does not contain lysine. In certain embodiments, a pharmaceutical composition comprising a GLP-1/glucagon agonist peptide (e.g., MEDI0382) does not contain trehalose. In certain embodiments, a pharmaceutical composition comprising a GLP-1/glucagon agonist peptide (e.g., MEDI0382) does not contain sucrose. In certain embodiments, a pharmaceutical composition comprising a GLP-1/glucagon agonist peptide (e.g., MEDI0382) does not contain magnesium chloride. In certain embodiments, a pharmaceutical composition comprising a GLP-1/glucagon agonist peptide (e.g., MEDI0382) does not contain histidine. In certain embodiments, a pharmaceutical composition comprising a GLP-1/glucagon agonist peptide (e.g., MEDI0382) does not contain arginine. In certain embodiments, a pharmaceutical composition comprising a GLP-1/glucagon agonist peptide (e.g., MEDI0382) does not contain glutamic acid. In certain embodiments, a pharmaceutical composition comprising a GLP-1/glucagon agonist peptide (e.g., MEDI0382) does not contain lysine, trehalose, sucrose, magnesium chloride, histidine, arginine, and/or glutamic acid. In certain embodiments, a pharmaceutical composition comprising a GLP-1/glucagon agonist peptide (e.g., MEDI0382) does not contain an amino acid.

In certain embodiments, a pharmaceutical composition comprising a GLP-1/glucagon agonist peptide (e.g., MEDI0382) comprises about 190 mM to about 270 mM sorbitol and about 0.2% to about 0.5% m-cresol. In certain embodiments, a pharmaceutical composition comprising a GLP-1/glucagon agonist peptide (e.g., MEDI0382) comprises about 190 mM to about 270 mM sorbitol and up to 30 mM sodium phosphate. In certain embodiments, a pharmaceutical composition comprising a GLP-1/glucagon agonist peptide (e.g., MEDI0382) comprises about 0.2% to about 0.5% m-cresol and up to 30 mM sodium phosphate. In certain embodiments, a pharmaceutical composition comprising a GLP-1/glucagon agonist peptide (e.g., MEDI0382) comprises about 190 mM to about 270 mM sorbitol, about 0.2% to about 0.5% m-cresol and/or up to 30 mM sodium phosphate. In certain embodiments, the pH is at least 7.9, e.g., about 8.1.

In certain embodiments, a pharmaceutical composition comprising a GLP-1/glucagon agonist peptide (e.g., MEDI0382) comprises about 35 mg/mL to about 45 mg/mL sorbitol and about 2.7 mg/mL to about 4.5 mg/mL m-cresol. In certain embodiments, a pharmaceutical composition comprising a GLP-1/glucagon agonist peptide (e.g., MEDI0382) comprises about 35 mg/mL to about 45 mg/mL sorbitol and up to 10 mg/mL sodium phosphate. In certain embodiments, a pharmaceutical composition comprising a GLP-1/glucagon agonist peptide (e.g., MEDI0382) comprises about 2.7 mg/mL to about 4.5 mg/mL m-cresol and up to 10 mg/mL sodium phosphate. In certain embodiments, a pharmaceutical composition comprising a GLP-1/glucagon agonist peptide (e.g., MEDI0382) comprises about 35 mg/mL to about 45 mg/mL sorbitol, about 2.7 mg/mL to about 4.5 mg/mL m-cresol, and up to 10 mg/mL sodium phosphate. In certain embodiments, the pH is at least 7.9, e.g., about 8.1.

In certain embodiments, a pharmaceutical composition comprising a GLP-1/glucagon agonist peptide (e.g., MEDI0382) comprises about 220 mM or about 220.3 mM sorbitol, about 20 mM or about 20.1 mM, sodium phosphate (e.g., a mixture of sodium phosphate monobasic monohydrate and sodium phosphate dibasic heptahydrate), and about 0.31% w/v meta-cresol, and a pH of about 8.1. In certain embodiments, the pharmaceutical composition comprises about 1 mg/mL of the GLP-1/glucagon agonist peptide (e.g., MEDI0382). In certain embodiments, the pharmaceutical composition further comprises sodium hydroxide.

In certain embodiments, a pharmaceutical composition comprising a GLP-1/glucagon agonist peptide (e.g., MEDI0382) comprises about 220 mM or about 220.3 mM sorbitol, about 10 mM sodium phosphate (e.g., sodium phosphate dibasic heptahydrate), and about 0.31% w/v meta-cresol, and a pH of about 8.1. In certain embodiments, the pharmaceutical composition comprises about 1 mg/mL of the GLP-1/glucagon agonist peptide (e.g., MEDI0382).

In certain embodiments, a pharmaceutical composition comprising a GLP-1/glucagon agonist peptide (e.g., MEDI0382) comprises about 220 mM or about 220.3 mM sorbitol, about 20 mM or about 20.1 mM sodium phosphate (e.g., a mixture of sodium phosphate monobasic monohydrate and sodium phosphate dibasic heptahydrate), and about 0.4% w/v meta-cresol, and a pH of about 8.1. In certain embodiments, the pharmaceutical composition comprises about 1 mg/mL of the GLP-1/glucagon agonist peptide (e.g., MEDI0382). In certain embodiments, the pharmaceutical composition further comprises sodium hydroxide.

In certain embodiments, a pharmaceutical composition provided herein is contained in a pen, e.g., a multi-dose pen. In certain embodiments, a pharmaceutical composition provided herein is contained in a syringe, e.g. a multi-dose syringe. In certain embodiments, a pharmaceutical composition provided herein is contained in a vial, e.g., a glass vial. The vial, e.g., glass vial, can be a multi-dose vial.

In certain embodiments, a pharmaceutical composition provided herein is physically stable. In certain embodiments, a pharmaceutical composition provided herein is chemically stable. In certain embodiments, a pharmaceutical composition provided herein is physically stable and chemically stable. In certain embodiments, a pharmaceutical composition provided herein does not form high order aggregates. In certain embodiments, a pharmaceutical composition provided herein does not show an increase in fibril formation. In certain embodiments, Staphylococcus aureus does not grow in a pharmaceutical composition provided herein after 28 days at room temperature. In certain embodiments, Escherichia coli does not grow in a pharmaceutical composition provided herein after 28 days at room temperature. In certain embodiments, neither Staphylococcus aureus nor Escherichia coli grows in a pharmaceutical composition provided herein after 28 days at room temperature.

### V. Methods of Treating

GLP-1/glucagon agonist peptides (e.g., MEDI0382) can combine the effect of glucagon e.g., inhibition of food intake or regulation of glucose levels with the effect of GLP-1 e.g., inhibition of gastric motility, or promotion of insulin release. They can therefore act to accelerate elimination of excessive adipose tissue, induce sustainable weight loss, and improve glycemic control. GLP-1/glucagon agonist peptides (e.g., MEDI0382) can also act to reduce cardiovascular risk factors such as high cholesterol, and high LDL-cholesterol or abnormal HDL/LDL ratios.

This disclosure provides a method of treating obesity or an obesity-related disease or disorder, comprising administering to a subject in need of treatment a pharmaceutical composition provided herein comprising a GLP-1/glucagon agonist peptide (e.g., MEDI0382). In certain instances, the administration is an adjunct to diet and exercise. In certain instances, the subject has type 2 diabetes mellitus. In certain instances, the subject has a body mass index (BMI) of 30 to 39.9 kg/m². In certain instances, the subject has a BMI of at least 40.

This disclosure also provides a method of reducing body weight, comprising administering to a subject in need of treatment a pharmaceutical composition provided herein comprising a GLP-1/glucagon agonist peptide (e.g., MEDI0382). In certain instances, the administration is an adjunct to diet and exercise. In certain instances, the subject has type 2 diabetes mellitus. In certain instances, the subject has a BMI of 27 to 40 kg/m′. In certain instances, the subject has a BMI of 30 to 39.9 kg/m². In certain instances, the subject has a BMI of at least 40. In certain instances, the subject is overweight. In certain instances, the subject is obese.

This disclosure also provides a method of reducing body fat, comprising administering to a subject in need of treatment a pharmaceutical composition provided herein comprising a GLP-1/glucagon agonist peptide (e.g., MEDI0382). In certain instances, the administration is an adjunct to diet and exercise. In certain instances, the subject has type 2 diabetes mellitus. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese. In certain instances, the fat is liver fat.

This disclosure also provides a method of treating Nonalcoholic Steatohepatitis (NASH), comprising administering to a subject in need of treatment a pharmaceutical composition provided herein comprising a GLP-1/glucagon agonist peptide (e.g., MEDI0382). In certain instances, the administration is an adjunct to diet and exercise. The administration can also reduce body weight or treat obesity. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese.

This disclosure also provides a method of treating Nonalcoholic Fatty Liver Disease (NAFLD), comprising administering to a subject in need of treatment a pharmaceutical composition provided herein comprising a GLP-1/glucagon agonist peptide (e.g., MEDI0382). In certain instances, the administration is an adjunct to diet and exercise. The administration can also reduce body weight or treat obesity. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese.

This disclosure also provides a method of reducing liver fat comprising administering to a subject in need of treatment a pharmaceutical composition provided herein comprising a GLP-1/glucagon agonist peptide (e.g., MEDI0382). In certain instances, the administration is an adjunct to diet and exercise. The administration can also reduce body weight or treat obesity. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese.

As provided herein, a pharmaceutical composition provided herein comprising a GLP-1/glucagon agonist peptides (e.g., MEDI0382) can be administered for preventing weight gain, preventing fat gain (e.g., liver fat), promoting weight loss, promoting fat loss (e.g., liver fat), reducing excess body weight, reducing fat (e.g., liver fat), or treating obesity (e.g. by control of appetite, feeding, food intake, calorie intake, and/or energy expenditure), including morbid obesity. This disclosure also provides a method of treating or preventing a disease or condition caused or characterized by excess body weight or excess body fat, comprising administering to a subject in need of treatment a pharmaceutical composition provided herein comprising a GLP-1/glucagon agonist peptide (e.g., MEDI0382). In certain instances, the administration is an adjunct to diet and exercise. In addition, a pharmaceutical composition provided herein comprising a GLP-1/glucagon agonist peptides (e.g., MEDI0382) can be used for treatment of other obesity-related metabolic disorders. Examples of other obesity-related (excess body weight-related) disorders include without limitation: insulin resistance, glucose intolerance, pre-diabetes, increased fasting glucose, type 2 diabetes, hypertension, dyslipidemia (or a combination of these metabolic risk factors), glucagonomas, cardiovascular diseases such as congestive heart failure, atherosclerosis, arteriosclerosis, coronary heart disease, or peripheral artery disease, stroke, respiratory dysfunction, or renal disease.

This disclosure also provides a method of treating type 2 diabetes mellitus, comprising administering to a subject in need of treatment a pharmaceutical composition provided herein comprising a GLP-1/glucagon agonist peptide (e.g., MEDI0382). In certain instances, the administration is an adjunct to diet and exercise. The administration can also reduce body weight or treat obesity. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese.

This disclosure also provides a method of improving glycemic control, comprising administering to a subject in need of treatment a pharmaceutical composition provided herein comprising a GLP-1/glucagon agonist peptide (e.g., MEDI0382). In certain instances, the administration is an adjunct to diet and exercise. The administration can also reduce body weight or treat obesity. In certain instances, the subject has type 2 diabetes mellitus. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese.

In certain embodiments, the route of administration of a pharmaceutical composition provided herein comprising a GLP-1/glucagon agonist peptide (e.g., MEDI0382) is parenteral. In certain embodiments, the route of administration of a pharmaceutical composition provided herein comprising a GLP-1/glucagon agonist peptide (e.g., MEDI0382) is subcutaneous. In certain embodiments, a pharmaceutical composition provided herein comprising a GLP-1/glucagon agonist peptide (e.g., MEDI0382) is administered by injection. In certain embodiments, a pharmaceutical composition provided herein comprising a GLP-1/glucagon agonist peptide (e.g., MEDI0382) is administered by subcutaneous injection.

In certain instances, a pharmaceutical composition provided herein comprising a GLP-1/glucagon agonist peptide (e.g., MEDI0382) can be administered once per day. In certain instances, a pharmaceutical composition provided herein comprising a GLP-1/glucagon agonist peptide (e.g., MEDI0382) can be administered once per day via injection (e.g., subcutaneous administration). In certain instances, a pharmaceutical composition provided herein comprising a GLP-1/glucagon agonist peptide (e.g., MEDI0382) can be administered once per day via injection (e.g., subcutaneous administration) over a period of at least one week, over a period of at least two weeks, over a period of at least three weeks, or over a period of at least four weeks.

### VI. Kits

In yet other embodiments, the present disclosure provides kits comprising the pharmaceutical compositions of GLP-1/glucagon agonist peptides described herein. In certain embodiments, a kit comprises a GLP-1/glucagon agonist peptide composition disclosed herein in one or more containers. One skilled in the art will readily recognize that the disclosed GLP-1/glucagon agonist peptide compositions can be readily incorporated into one of the established kit formats which are well known in the art.

### EXAMPLES

### Example 1: MEDI0382 Solubility and pH Stability Profile

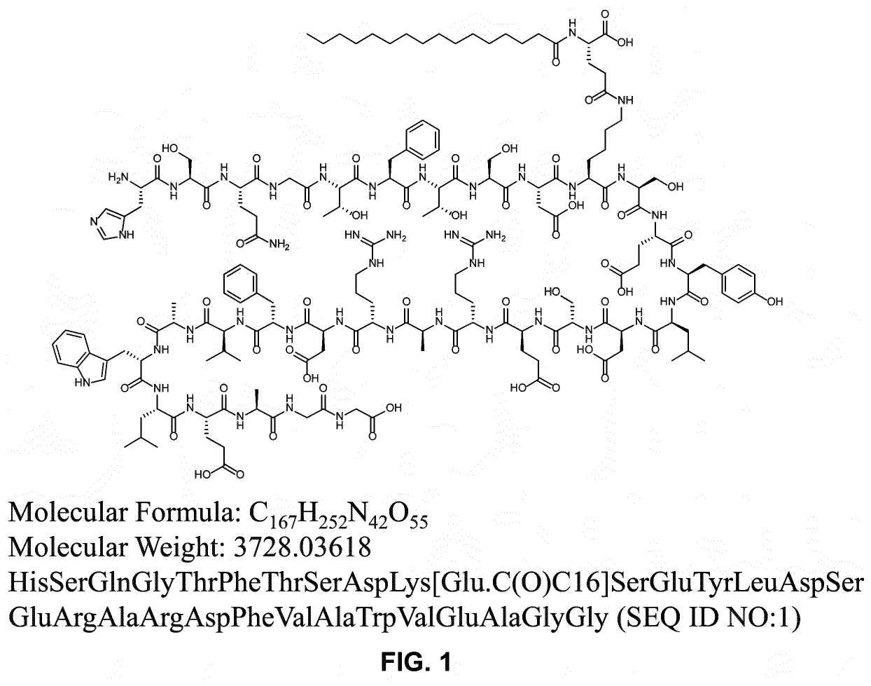

The solubility and pH stability of the GLP-1/glucagon agonist peptide MEDI0382 (FIG. 1) were studied. In particular, solubility was studied in different types of buffers, different buffer ionic strengths, pure water, and organic solvents. The aqueous pH solubility of MEDI0382 was also assessed. The chemical stability (as measured by reverse phase ultra performance liquid chromatography (RP-UPLC)) and secondary structure (as measured by Fourier transform infrared spectroscopy (FTIR) and UV circular dichoism spectroscopy (CD)) were also assessed at various pHs. Furthermore, the effect of pH on the kinetics of MEDI0382 aggregation were also assessed in a thioflavin T binding (ThT) assay. The results of these experiments are summarized in Table 2.

TABLE 2
Summary of MEDI0382 solubility and pH stability studies

| Attribute | Description |
|---|---|
| Physical state | Amorphous powder, hygroscopic, sensitive |
| | to light |
| Predicted isoelectric point | pH(I) = 4.2 |
| Solubility isoelectric point | pH(I) = 4.1-4.5 |
| Predicted molecular charge at | −4 |
| pH = 8 | |
| Preferable buffers solubility | Sodium phosphate and TRIS |
| Ranges of ionic strength solubility | ≥25 mM |
| Organic solvent solubility | Low |
| Aqueous solution pH solubility | Insoluble at pH 4.1 to 4.5, solubility |
| | increases at pH 1 to 3 and 6 to 13 |
| Aqueous solution chemical pH | Stability ranking: pH 3~6~7~8 >1.4 >>> 12 |
| stability | Opalescence and “sticking” at pH 1.4 |
| | No physical changes at pH 7 to 8 |
| | Overall better stability at pH 7 to 8 |
| pH impact on secondary structure | Favors β-sheet and random coil at pH 6 and |
| | increases α-helix in alkaline conditions (7 to 8) |
| pH impact on aggregation kinetics | Alkaline conditions improve aggregation |
| | kinetics (higher lag time at pH 8 (93 hours), |
| | lower lag time at pH 6 (hours) |

The studies in Table 2 were key to guiding the selection of the buffer and pH formulation taken into further development.

### Example 2: Excipient Screen

Based on the results of the solubility and pH stability studies, attention was paid to the compatibility of MEDI0382 with generally recognized as safe (GRAS) excipients for liquid formulation that could be administered by subcutaneous route. The stability of MEDI0382 in fifteen (15) different formulations was assessed by Dynamic Light Scattering (DLS) method. The results are summarized in Table 3.

TABLE 3
Summary of chemical and physical stability of GRAS excipient screen

| | | Total | | |
|---|---|---|---|---|
| | | Purity loss | Increase in | |
| | | by RP-UPLC | Particle size | |
| Formulation | Formulation | method at | by DLS at | Stability |
| number | Composition | 40° C./week | 40° C./week | ranking |
| 1 | PB¹ 25 mM, PG² 1.85% | <3% | >10 nm * at | MEDIUM |
| | (w/v), pH 7.1 | | 5° C. | |
| 2 | PB 50 mM, PG 1.85% | <3% | <10 nm | ACCEPTABLE |
| | (w/v), pH 7.3 | | | |
| 3 | PB 100 mM, PG 1.85% | <3% | <10 nm | ACCEPTABLE |
| | (w/v), pH 7.2 | | | |
| 4 | PB 50 mM, 100 mM | <3% | >100 nm | HIGH |
| | Arginine, pH 7.2 | | (2 wk) | |
| 5 | PB 50 mM, 50 mM | <3% | >100 nm | HIGH |
| | Arginine + 50 mM | | (2 wk) | |
| | Glycine, pH 6.5 | | | |
| 6 | PB 50 mM, Sorbitol 3% | <3% | <10 nm | MEDIUM |
| | (w/v), pH 7.1 | | | |
| 7 | PB 50 mM Sorbitol | <3% | >10 nm | ACCEPTABLE |
| | 1.3% (w/v), pH 7.1 | | | |
| 8 | PB 50 mM, Glycerol | <3% | <10 nm | ACCEPTABLE |
| | 0.7% (w/v) + PG | | | |
| | 1.85% (w/v), pH 7.1 | | | |
| 9 | TRIS 100 mM, 150 mM | >5% | <10 nm | HIGH |
| | sorbitol, pH 7.2 | | | |
| 10 | TRIS 100 mM, 150 mM | <3% | <10 nm | ACCEPTABLE |
| | Mannitol, 10 mM | | | |
| | methionine, pH 7.2 | | | |
| 11 | TRIS 100 mM, 300 mM | <3% | <10 nm | MEDIUM |
| | Mannitol, pH 7.2 | | | |
| 12 | TRIS 100 mM, 150 mM | <3% | <10 nm | ACCEPTABLE |
| | Mannitol, 20 mM | | | |
| | methionine, pH 7.2 | | | |
| 13 | TRIS 50 mM, 150 mM | <3% | <10 nm | MEDIUM |
| | Mannitol, pH 6.5 | | | |
| 14 | TRIS 50 mM, 150 mM | <3% | <10 nm | ACCEPTABLE |
| | Mannitol, 10 mM | | | |
| | methionine, pH 7.2 | | | |
| 15 | PB 100 mM, PG | <3% | <10 nm | ACCEPTABLE |
| | 1.85% (w/v), 10 mM | | | |
| | methionine, pH 7.2 | | | |

¹ PB = sodium phosphate buffer
² PG = propylene glycol

In accelerated conditions, the formulation containing phosphate buffer, arginine, and glycine (pH 6.5) showed a dramatic increase in the Z-average values indicated less physical stability. The experimental pI of MEDI0382 is 4.1 to 4.5. The preferred buffer options were TRIS and sodium phosphate, and the most acceptable excipients were mannitol, propylene glycol, and sorbitol.

The principles for formulation ranking used in Table 3 were the loss of peptide purity (main area peak/total peak area) at accelerated conditions (purity loss≤3% per 1 week at 40° C., low risk; purity loss>5% week at 40° C.=high risk) and physical stability at 40 or 5° C. (Z-average<10 nm=low risk, >10 nm=medium risk; >100 nm=high risk). The stability ranking indicates lower stability performance with Arginine (formulation 4 and 5), sorbitol 3% w/v (formulation 9), low sodium phosphate buffer ionic strength (formulation 1) and pH below 7.0 (formulations 5 and 13).

Overall, the results of this study point toward sodium phosphate (>25 mM) and TRIS buffer, propylene glycol, glycerol, methionine, mannitol, and pH≥7.

### Example 3: One-Month and Three-Month Stability Studies

Six formulations were designed to be taken forward in development. These formulations are provided in Table 4.

TABLE 4

| Formulation | Composition | pH |
|---|---|---|
| 1 | TRIS 100 mM, 150 mM mannitol | 7.2 ± 0.2 |
| 2 | TRIS 100 mM, 150 mM Mannitol, | 7.2 ± 0.2 |
| | 10 mM methionine | |
| 3 | TRIS 100 mM, 150 mM Mannitol, | 7.2 ± 0.2 |
| | 20 mM methionine | |
| 4 | PB 50 mM, PG 1.85% (w/v) | 7.2 ± 0.2 |
| 5 | PB 50 mM, Glycerol 0.7% (w/v); | 7.2 ± 0.2 |
| | PG 1.85% (w/v) | |
| 6 | PB 50 mM, PG 1.85% (w/v), | 7.0 ± 0.2 |
| | 10 mM methionine | |

The six selected formulations were used in a 1-month stability study at refrigerated (2-8° C.) and stressed conditions (37° C.). Samples were assessed at time zero, 2 weeks, and 4 weeks by RP-UPLC, DLS, Visual Inspection, Micro-Flow Imaging (MFI), Size Exclusion Chromatography-Multi-Angle Light Scattering (SEC-MALS).

The rates of degradation as measured by RP-UPLC were temperature dependent. When stored at 5° C., all formulations showed purity levels of ≥96.5% within 4 weeks. However, at 37° C., formulation 1 (TRIS, mannitol) shows significantly higher degradation rates (purity levels of ≥80%) compared to the others (purity levels of ≥90%). There was no significant change on the SEC MALS profile within 4 weeks (5° C.). The higher order structure was a trimer even at stressed conditions.

The stability study suggests that all six of the formulations tested have no significant physicochemical instability changes within 4 weeks, when stored at 5° C. Formulation 1 (“DF”: TRIS 100 mM, mannitol 150 mM) was significantly less stable at thermal stressed conditions. Formulation 2 (DF+methionine) showed significant improvement on the thermal stability at forced conditions (37° C.). Formulation 2 (DF+methionine) showed significant improvement on the thermal stability at forced conditions (37° C.). Overall formulations 2, 4 and 6 demonstrate marginal better stability profile in stressed conditions.

Formulations 2, 4 and 6 were selected for the pK study in the following example based on the stressed conditions data.

Along with the one-month study, a three-month stability study was carried out using the Formulation 1 (Default formulation, TRIS 100 mM/Mannitol 150 mM, pH 7.2±0.2).

Formulation 1 was selected for the pharmacokinetic (PK) study in the following example based on the three-month data at 5° C. and for comparability with previous pre-clinical studies (MsC).

### Example 4: PK Study of Formulations

A PK analysis was conducted in rats (0.1 mg/kg subcutaneously (sc or SQ)) using Formulations 1, 2, 4, and 6 selected from the previous example. The PK analysis suggested a comparable half-life and bioavailability for all four formulations tested. However, formulation 2 (TRIS, mannitol, methionine) had slightly lower bioavailability than the others. In view of these similarities, the chemical stability profiles were used to prioritize the formulations. Formulation 2 (sodium phosphate 50 mM, propylene glycol 1.85% (w/v), 5 mg/mL, pH 7.2±0.3) showed the best performance. Formulation 1 was kept for further comparability studies, and Formulation 4 demonstrated reduced oxidation levels.

### Example 5: Long-Term Stability Studies

Three formulations, all containing MEDI0382 at a concentration of 5 mg/ml were selected for the long term stability study. These formulations are provided in Table 5.

TABLE 5
MEDI0382 (5 mg/mL) Formulations for Long-Term
Stability Studies

| Formulation | Composition | pH |
|---|---|---|
| 1 | TRIS 100 mM, 150 mM Mannitol (DF) | 7.37 |
| 2 | TRIS 100 mM, 150 mM Mannitol, | 7.37 |
| | 20 mM methionine | |
| 3 | PB 50 mM, PG 1.85% (w/v) | 7.35 |

Purity (RP-UPLC), aggregation (DLS), peptide concentration (A280), High order structure (SEC-MALS)(multiangle (laser) light scattering), Sub-visible particles (MFI), Osmolarity, Conformational stability (Circular dichroism (CD) spectroscopy) and pH tests were conducted. The following temperatures were tested: 5° C., 15° C.; 25° C., 40° C. and −80° C. The following time points were tested: 0, 2 weeks, 1, 2, 3, 6, 9, 12, 24 and 36 months.

There was a low amount of MEDI0382 degradation per month (0.3% or lower). Degradation routes included possible isomerization, deamidation, oxidation, and fragmentation as elucidated by LC-MS based on 40° C. data. As measured by RP-UPLC degradation was most for Formulation 3 and least for Formulation 1.

Dynamic light scattering (DLS) assessment of aggregation shows that there was no significant change in aggregation at low temperatures (5° C. to 25° C.). However, there was an upsurge in the Z-average values at forced conditions (40° C.) that was more significant in Formulation 2.

Micro-Flow imaging (MFI) showed low levels of sub-visible particle formation in the range of ≥10 μm and 25 μm for all formulations and conditions. SEC-MALS results indicated that oligomeric forms tend to remain stable over 3 months. Formulation 1 showed a steady dip from a tetramer to a trimer (thus indicating a change in the oligomeric state), but the observation may have been related to the variability of the method.

There were no significant changes on peptide concentration, osmolarity, or pH within 3 months at all tested conditions.

In sum, all 3 formulations showed enough stability at 5° C. and −80° C. within 3 months. The rates of degradation were highly dependent on the temperature, but the kinetics of degradation were very similar for the 3 different formulations. The Rat PK (SQ) of the 3 formulations shows very similar profile. Formulation 3 demonstrated slightly better stability compared to Formulations 1 and 2.

However, at the 6-month time point, the physical stability data obtained by DLS and MFI suggest that an aggregation process may have started. The DLS data showed a sharp growth on the particle size over time that was dependent on temperature. Furthermore, the MFI analysis indicated a growth of the number of particles between 1 to 2 μm at 5 and −80° C. These results indicated an aggregation/particle formation risk for both liquid and frozen formulations after 6 months stored in refrigerated conditions (2-8° C.).

### Example 6: Non-GLP Toxicology Studies

The chemical stability of MEDI0382 was tested in 50 mM Phosphate buffer, 1.85% (w/v) of propylene glycol, pH 7.4. It was stable under freeze thaw stress conditions, which indicated that acceptable levels of total degradation occurred over a 1 week period at 5° C. after 3 freeze thaw stress cycles. The same studies showed formulation physical stability by Dynamic Light Scattering (DLS) and visual inspection over the same period.

PES Millex-GP Filter (0.22 μm) is the recommended filter for formulation sterilization.

This MEDI0382 formulation was stable in BD Plastipak syringes for at least 4 hours at room temperature.

However, an adsorption of the peptide to the containers at low levels of dosing (below 0.1 mg/ml) was identified. This MEDI0382 formulation was ruled out because the low doses of drug likely to be required to achieve a pharmacological (agonistic) effect could not feasibly be delivered in this way.

### Example 7: Adsorption Control Assessment

Given the low dose requirements needed for MEDI0382 and container closure surfaces adsorption issues, addition of surfactant polysorbate 80 was studied. The formulation was as follows: 50 mM sodium phosphate buffer (PB) pH 7.5 containing 1.85% w/v propylene glycol (PG) and 0.03% v/v polysorbate 80 (PS80), MEDI0382 5 mg/mL or 2 mg/mL.

Samples were stressed by temperature and freeze/thaw and analyzed for purity, aggregation, visual inspection, and for the presence of sub-visible particles.

RP-UPLC was used to asses purity of the formulation over 2 months at 5, 25, 32 and 40° C. The results show a temperature stability dependence, with higher temperatures resulting in decreased purity.

After one month, the physical stability was measured by DLS. The Z-average values increased sharply over the month at all temperatures, except at −80° C., suggesting high levels of aggregation. In line with the DLS findings, visual inspection showed a gel-like-appearance after 2-month storage. In summary, all techniques indicated physical instability of the formulation at all temperatures except −80° C.

In the freeze/thaw studies, the formulation was tested in the presence or absence of polysorbate 80 (0.3% v/v) with either 2 mg/ml or 5 mg/ml protein. Fresh solutions (glass vials, Type I, transparent) were frozen by three freeze/thaw cycles. After the thawing, samples were kept at 5 and 25° C. for up to 3 weeks and analysed by visual inspection. Samples without freezing were run in parallel as control. In a second study, the container closer compatibility was assessed by using Nalgene HDPE (more suitable for toxicological studies). In this case, only formulation at 5 mg/mL was analysed.

Taking together both the liquid and freeze/thaw stress studies, the data shows that polysorbate 80 has an effect on the physical stability (gel-like appearance) of the formulation. The peptide concentration, temperature, and the freeze/thaw stress are the most relevant identified factors regulating the physical stability of this MED0382 formulation.

### Example 8: 9-Month Stability Study

The stability of two 5 mg/ml MEDI0382 formulations was analyzed over 9 months. The first formulation contained 50 mM Phosphate buffer; 1.85% (w/v) and Propylene Glycol, pH 7.4, and the second formulation contained 100 mM TRIS, 150 mM Mannitol, 20 mM methionine pH 7.4. The two different formulations show very similar loss of purity within 9 months.

In another 9-month study, the stability of 3 MEDI0382 formulations was studied in refrigerated conditions. The first formulation contained 100 mM TRIS, 150 mM Mannitol, pH 7.4. The second formulation contained 100 mM TRIS, 150 mM Mannitol, 20 mM Methionine, and the third formulation contained 50 mM Phosphate buffer; 1.85% (w/v) Propylene Glycol, pH 7.4. The formulations were analysed by DLS and MFI. The third formulation showed a sharp increase in Z-average particle size (DLS) from 3 to 9 months. A similar pattern was observed with sub-visible particles by MFI analysis, suggesting high aggregation level and sub-visible particle formation.

In sum, although acceptable chemical stability was observed, high levels of aggregation were identified from the 6-month time point. Fibrillation could be a mechanism of aggregation.

### Example 9: Decreasing MEDI0382 Protein Concentration to 2 mg/mL

In order to improve the formulation shelf-life, formulations with 2 mg/mL MEDI0382 were evaluated. The chemical stability of the 2 mg/mL and 5 mg/mL formulations were very similar, but decreasing the peptide concentration did not improve physical stability. A sharp increase in total sub-visible particles was observed by MFI at 6 months for the 2 mg/ml formulation and at 12 months for the 5 mg/mL formulation. Similarly, DLS analysis showed an increase in Z-average from 6 month for the 2 mg/mL formulation and at 12 months for the 5 mg/mL formulation. Two of the five samples at 5 mg/mL turned to gel at month 13, and at month 18, the other three 5 mg/mL samples turned to gel. None of the 2 mg/mL samples turned to gel until 18 months.

The pH stability of 2 mg/ml formulations were also studied at pH of 7.0, 7.5, and 8.0 in glass vials. The formulations were then placed in stability at 5° C. and 40° C. for three months. Samples were analyzed by RP-UPLC, DLS, and AFM. Increased aggregation levels and purity loss were observed at pH 7 to 7.5. Physicochemical stability was improved by increasing the pH to 7.8.

The chemical stability of both the 2 and 5 mg/mL formulations were within acceptable levels for human trials as a liquid drug product (≥94% at 12 months). The presence of polysorbate 80 impacts the physical stability (gel-like appearance) of the formulation. Peptide concentration, temperature, and freeze/thaw stress are the most relevant identified factors playing an effect on physical stability of MED0382 containing PS80. Temperature shows different effects on the time that it takes to form the gel-like appearance depending on the type of container closure. In the case of glass vial, gel-like formation is accelerated by low temperatures (5° C.). In contrast, for formulations stored in Nalgene HDPE, gelation is accelerated by higher temperatures. The physical stability of MEDI0382 is highly sensitive to the final formulation pH. Increased physical stability is achieved by rising the pH from 7 to 7.8. The mechanism of aggregation of MEDI0382 is the polymerization of the peptide to fibrils. Changing MEDI0382 concentration from 5 mg/mL to 2 mg/mL (without PS80) does not improve aggregation kinetics. Formulation at 2 mg/mL and pH 7.8 does not form fibrils up to 7-months storage at refrigerated conditions. The GMP stability of cycle formulation stability is at least 30 months at refrigerated conditions.

A formulation containing 50 mM sodium phosphate buffer (PB) pH 7.8 containing 1.85% w/v propylene glycol (PG), MEDI0382 2 mg/mL Liquid drug product (DP) for glass vial and syringe administration was recommended for administration for humans. The formulation recipe is shown in Table 6 below.

TABLE 6
Formulation Buffer Recipe targeting pH 7.8,
2 mg/mL MEDI0382 strength

| | | Molecular | Amount |
|---|---|---|---|
| | | weight | required |
| Constituents | Formula | (Daltons) | (g/Kg) |
| Sodium | NaH₂PO₄•H₂O | 137.99 | 0.5 |
| Phosphate, | | | |
| monobasic, | | | |
| monohydrate | | | |
| Sodium | Na₂HPO₄•7H₂O | 268.07 | 12.34 |
| Phosphate, | | | |
| dibasic, 7 | | | |
| hydrate | | | |
| Propylene | C₃H₈O₂ | 76.09 | 18.35 |
| Glycol(PG) | | | |
| Water | H₂O | 18.0 | To 1 kg |
| MEDI0382 | C₁₆₇H₂₅₂N₄₂O₅₅ | 3728.04 | 2.08 |

### Example 10: Decreasing MEDI0382 Protein Concentration to 0.5 mg/mL

A further decrease in MEDI0382 protein concentration was desirable based on the intended amount of drug to be administered to human patients. Thus, 0.5 mg/mL formulations compatible with pre-filled syringe (PFS) use were evaluated. Formulations containing 50 mM sodium phosphate buffer pH 7.8, 1.85% w/v propylene glycol, and MEDI0382 were placed in three different conditions: (i) in glass vial at 2 mg/mL, (ii) in glass vial at 0.5 mg/mL, and (iii) in PFS at 0.5 mg/mL (into BD 29 PFS siliconized). The PFS had a 0.15 mL fill volume. The glass vials were hand filled with 2.0 mg/mL or 0.5 mg/mL MEDI0382 at 1.0 ml fill volumes. Stability was assessed at 5° C., 25° C., and 45° C. at time 0, 2 weeks, 4 weeks, 6 weeks, and 3 months.

The results showed comparable stability for all three different presentations and acceptable stability for administration to humans (purity≥95%.) However, more sub-visible particles (measured by MFI) were present in PFS formulations compared to glass vial formulations. The following formulation targeting 0.5 mg/mL MEDI0382 was devised.

TABLE 7
Formulation Buffer Recipe targeting pH 7.8,
0.5 mg/mL MEDI0382 strength

| | | Molecular | Amount |
|---|---|---|---|
| | | weight | required |
| Constituents | Formula | (Daltons) | (g/Kg) |
| Sodium | NaH₂PO₄•H₂O | 137.99 | 0.72 |
| Phosphate, | | | |
| monobasic, | | | |
| monohydrate | | | |
| Sodium | Na₂HPO₄•7H₂O | 268.07 | 11.87 |
| Phosphate, | | | |
| dibasic, 7 | | | |
| hydrate | | | |
| Propylene | C₃H₈O₂ | 76.09 | 18.35 |
| Glycol(PG) | | | |
| Water | H₂O | 18.0 | To 1 kg |
| MEDI0382 | C₁₆₇H₂₅₂N₄₂O₅₅ | 3728.04 | 0.505 |

### Example 11: Effect of Buffers and Excipients on Stability

It was desirable to develop a multi-use and variable dose pen for once-daily subcutaneous injection (up to 6 doses), wherein each pen could contain up to 30 doses, and each daily dose could be between 50 mcg and 300 mcg of MEDI0382. A preservative would be required to ensure microbial safety after the pen device is actuated. Therefore, additional formulation analysis was undertaken to develop this formulation. In particular, the impact of pH, buffer, and excipients in the presence of antimicrobials on the physical and chemical stability of MEDI0382 was evaluated with MEDI0382 at a 5 mg/mL concentration.

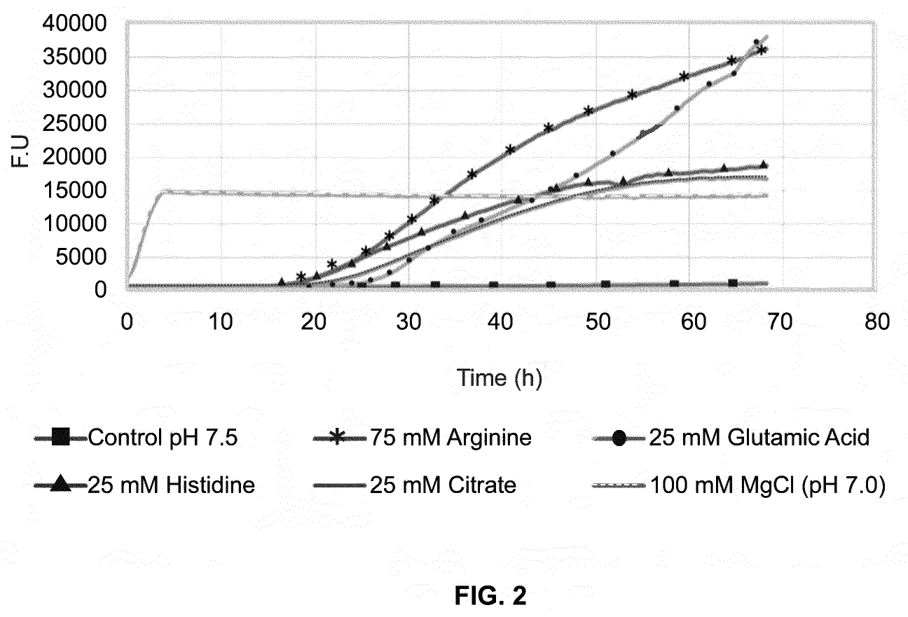

The effect of excipients on fiber formation was analyzed by evaluating whether or not amino acids and divalent ions could prevent fiber formation at pH 7.5. MEDI0382 was mixed with L-glutamic acid, citric acid, L-arginine, L-histidine, or magnesium chloride in 50 mM phosphate buffer at a final pH of 7.5. The fiber formation was evaluated using a Thioflavin T binding assay (ThT) assay. The results, shown in FIG. 2, demonstrate that none of the excipients stabilized MEDI0382. In fact, compared to the negative control, all of the excipients tested destabilized MEDI0382.

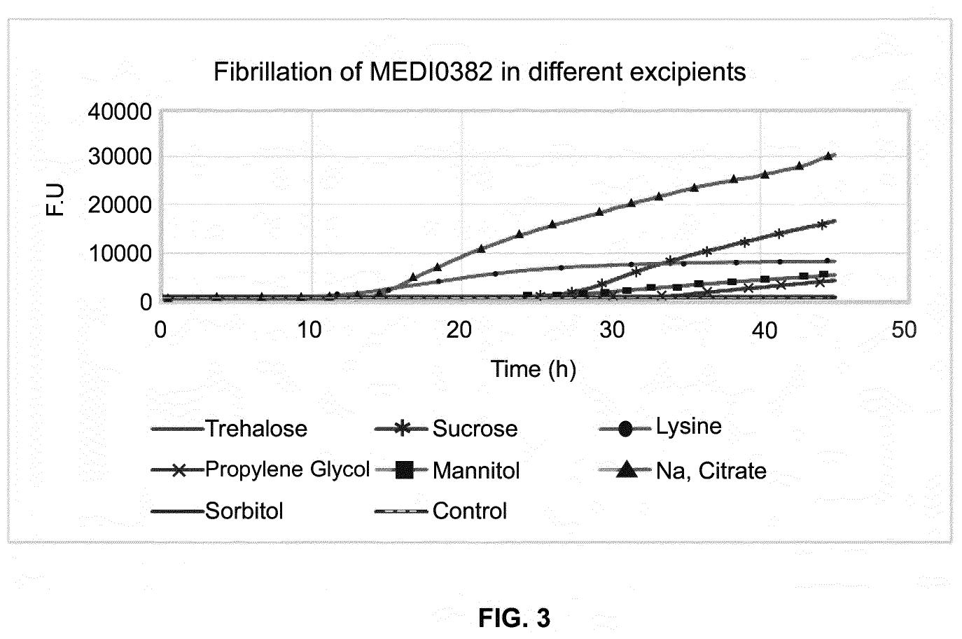

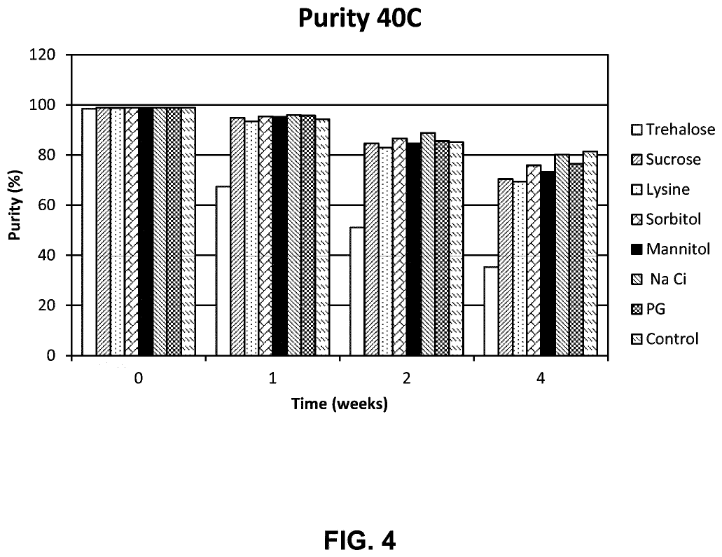

Another study was performed to understand the impact of trehalose, sucrose, L-lysine, sorbitol, mannitol, sodium citrate, and propylene glycol on MEDI0382 stability in the presence of 50 mM phosphate buffer at a final pH of 7.5. The fiber formation was again evaluated using a ThT assay. The results, shown in FIG. 3, demonstrate that mannitol, sorbitol, trehalose, and propylene glycol did not cause major increases in fibrillation (the sorbitol and trehalose data in FIG. 3 overlap with the control data), while sucrose, lysine, and sodium citrate increased instability. Chemical degradation associated with these excipients was also monitored for one month for samples kept at 5° C. and 40° C. using an RP-UPLC assay. The results demonstrated that trehalose significantly speeds chemical degradation. Both sorbitol and mannitol (97% purity at 5° C.) induced less chemical degradation than propylene glycol (95% purity) (See FIG. 4 for data at 40° C.). Particle formation associated with these excipients was also monitored by visual inspection after shaking stress (800 rpm for 4 hours). Trehalose, sorbitol, propylene glycol, and the control sample resulted in few particles. However, the citrate and lysate samples turned into a solid white gel and a soft gel, respectively, and the mannitol formulation turned cloudy.

Based on this series of experiments, sorbitol, mannitol, and propylene glycol provided the best results.

### Example 12: pH Dependence of Gel Formation of MEDI0382

Although polysorbate 80 is commonly used to prevent aggregation, it caused MEDI0382 to form gel rapidly in a formulation with a pH of 7.5. (See Example 7.) The gel formation at two other pHs was therefore tested: 6.8 and 8.3.

In these experiments, formulations with 0.03% polysorbate 80 were compared to formulations without polysorbate 80. The samples were incubated for 7 days at 50° C. The sample containing polysorbate 80 at pH 6.9 turned into a solid gel, whereas the sample at pH 8.3 did not. At this stage, 10 mM NaOH was added to the gelled sample, and the sample turned liquid again. All of the samples were then incubated for another 12 days. The sample at pH 6.9 without polysorbate 80 turned into a soft gel, but the others stayed liquid. These results demonstrate that polysorbate 80 can accelerate gel formation, but the effect is pH dependent and reversible.

### Example 13: Effect of Excipients on Stability in Presence of Antimicrobials

The physical stability of MEDI0382 has an optimal pH>7.8. Few preservatives are active at this pH range. Three sets of experiments were performed to evaluate the physical and chemical stability of MEDI0382 under accelerated conditions in different buffers and excipients in the preservatives phenol and m-cresol.

In one set of experiments, mannitol (0-300 mM) and propylene glycol (0-2%) were evaluated with m-cresol (0-0.3%) and phenol (0-1%) in 50 mM phosphate buffer at pH 8.0. The samples were incubated at 5° C. and 40° C., and the chemical purity was monitored for four weeks. Shaking studies were performed on all the samples and visual inspections were performed. The physical stability was assessed with a ThT assay.

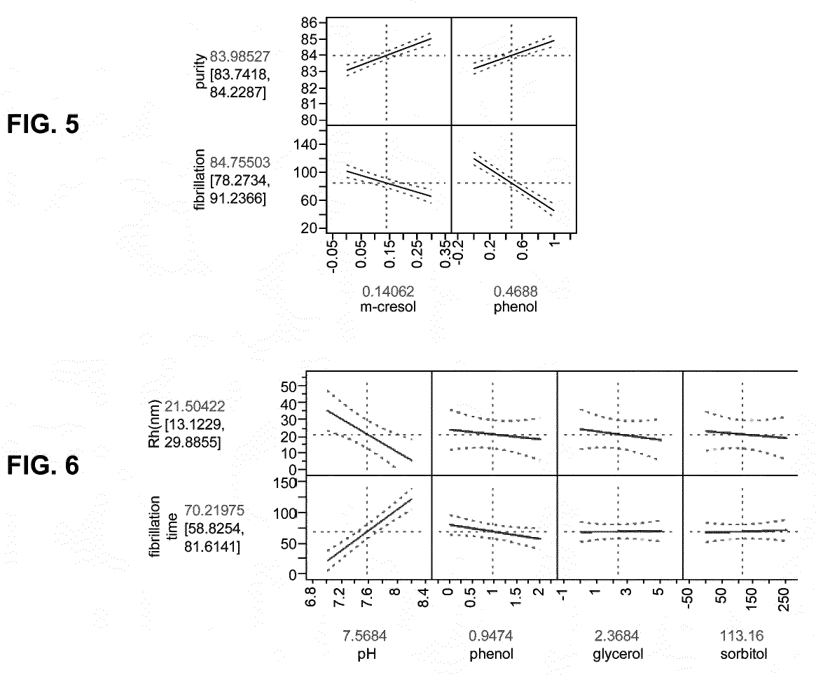

In these experiments, where the pH was kept constant at 8, opposite effects of the preservatives were observed on chemical and physical stability. (FIG. 5.) There was a slight negative effect of the preservatives on fibrillation, but a slight positive effect on the chemical stability. Mannitol and propylene glycol had no significant impact on purity or physical stability.

In another set of experiments, the impact of sorbitol (0-250 mM), propylene glycol (0-2%), and phenol (0-2%) in a pH range 7-8.2 on the physical stability of MEDI0382 was explored. Physical stability was monitored using DLS (DynaPro plate reader, Wyatt) and the ThT assay. The results, shown in FIG. 6, demonstrate that a high pH is favorable for the physical stability of MEDI0382 in the presence of all the excipients tested.

In a third set of experiments, the impact of sorbitol (0-250 mM), glycerol (0-5%), methionine (0-10 mM), and m-cresol (0-0.3%) in a pH range of 7-8.2 on the stability of MEDI0382 was explored. Chemical stability was monitored with RP-UPLC. Physical stability was assessed by ThT and shaking experiments. The chemical stability was monitored using RP-UPLC. The results of these experiments show a slight negative effect of m-cresol and a slight positive effect of methionine on physical stability.

Overall, the results from these three sets of experiments show that pH is the major factor contributing to both the physical and chemical stability of MEDI0382, and this could not be counteracted by any excipient or preservative tested. Both physical and chemical stability are improved at pH≥8. MEDI0382 is less stable in presence of lysine, trehalose, sucrose, sodium citrate, magnesium chloride (MgCl2), citrate, histidine, arginine, or glutamic acid. M-cresol or phenol showed a low impact on the physical or chemical stability of MEDI0382 in the presence of sorbitol, mannitol, propylene glycol, or glycerol, and adding up to 10 mM methionine could increase the physical stability of MEDI0382.

### Example 14: Long-Term Stability

The studies discussed in Example 13 identified potential excipients to maximize the stability of MEDI0382 using short term accelerated stability studies. However, the kinetics of MEDI0382 aggregation can be very slow, and stability issues such as particle formation and gelation can occur over long time periods. Therefore, seven (7) formulations were designed for long term stability studies.

Sorbitol, propylene glycol, and mannitol were selected as tonicity agents to assay. The product target profile for MEDI0382 is 290-300 mOsm/kg, and the concentration of the tonicity agent was adjusted accordingly to achieve that. Both phenol and m-cresol were assayed as preservatives. In addition, given the desirability of a pH of 8.1 for optimal stability, sodium phosphate was assayed as a buffering agent at a concentration of 20 mM. Sodium hydroxide was used to adjust the final pH of the formulation because sodium phosphate has a lower buffer capacity at pH 8.1 and the concentration of MEDI0382 has an impact on final pH formulation. The following seven formulations in Table 8 were assayed based on these criteria.

TABLE 8
Seven MEDI0382 (5 mg/mL) Formulations Evaluated for Long-Term Stability

| Formulation | Components |
|---|---|
| A | 20 mM Sodium phosphate (NaPi), 220 mM sorbitol, 0.5% phenol |
| | NaOH to pH 8.1 |
| B | 20 mM Sodium phosphate (NaPi), 220 mM sorbitol, 10 mM |
| | methionine, 0.5% phenol NaOH to pH 8.1 |
| C | 20 mM Sodium phosphate (NaPi), 220 mM sorbitol, 10 mM |
| | methionine, 0.3% m-cresol, NaOH to pH 8.1 |
| D | 20 mM Sodiumphosphate, 1.85% PG, 0.5% phenol, NaOH to pH 8.1 |
| E | 20 mM Sodium phosphate (NaPi), 10 mM citrate, 1.35% PG, 0.5% |
| | phenol, NaOH to pH 8.1 |
| F | 20 mM Sodium phosphate (NaPi), 1.85% PG, 0.3 m-cresol, NaOH to pH 8.1 |
| G | 220 mannitol, 0.5% phenol, NaOH to pH 8.1 |

Methionine (10 mM) was added in formulations A and B to evaluate its ability to increase the chemical stability of MEDI0382, and citrate (10 mM) was added in formulation E to evaluate its ability to act as an anti-microbial agent.

The seven formulations were prepared by dissolving MEDI0382 gently in 0.185 M sodium hydroxide (NaOH) to achieve a 2× final peptide concentration. A solution containing 2× concentration of all the other components of the final formulation was then added to the 2× MEDI0382 solution. The mixed solution was filtered, and the pH was adjusted with 0.1 M NaOH if needed. Placebo for each formulation A-G was also prepared for filling. The formulations were filled in cartridges and vials.

The primary container for MEDI0382 is a 3 ml cartridge (Ompi EZ fill cartridges, part no 70109079) with a permeable membrane and a rubber stopper (FORMULA ART22234023/50GRY). The cartridges were filled manually and stoppered using a manual stoppering tool. The fill volume was 3 mL.

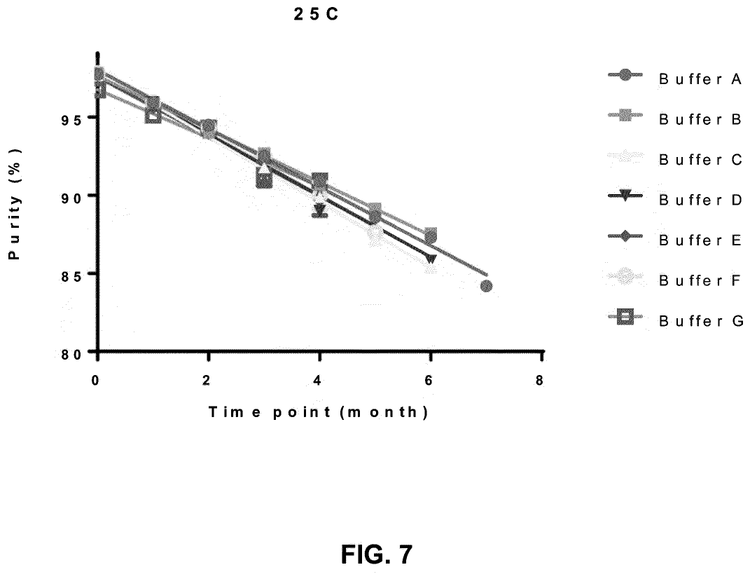

The purity of MEDI0382 in the seven different formulations (A-G) was monitored for 6 months at 25° C. and during 24 months at 5° C. The chemical degradation for the formulations stored at 25° C. did not reveal any differences between the formulations, and purity loss was consistent at about 2% per month. (FIG. 7)

For some of the formulations there were an increase in the hydrodynamic diameter, indicating that aggregation is occurring. In formulation D, E, and F the increase appeared between 9-12 months for cartridges stored at 5° C. (See Table 9 below.) Formulations D, E, and F all contain propylene glycol as the tonicity agent.

TABLE 9
Dh(d) of Seven MEDI0382 Formulations
in Cartridges Stored at 5° C.

| Dh(d) | | | | | |
|---|---|---|---|---|---|
| (nm) | T = 0 | T = 3 | T = 6 | T = 9 | T = 12 |
| A | 4.9 | 7.2 | 9.5 | 5.5 | 12.3 |
| B | 5.4 | 6.5 | 6.5 | 4.1 | 6.1 |
| C | 4.8 | 9.2 | 7.6 | 4.9 | 5.4 |
| D | 4.5 | 7.8 | 6.5 | n/a | 104.1 |
| E | 4.9 | 7.1 | 7.9 | n/a | 324.8 |
| F | 8.0 | 5.6 | 10.3 | 6.6 | 316.8 |
| G | 7.9 | 8.0 | 6.9 | n/a | 6.6 |

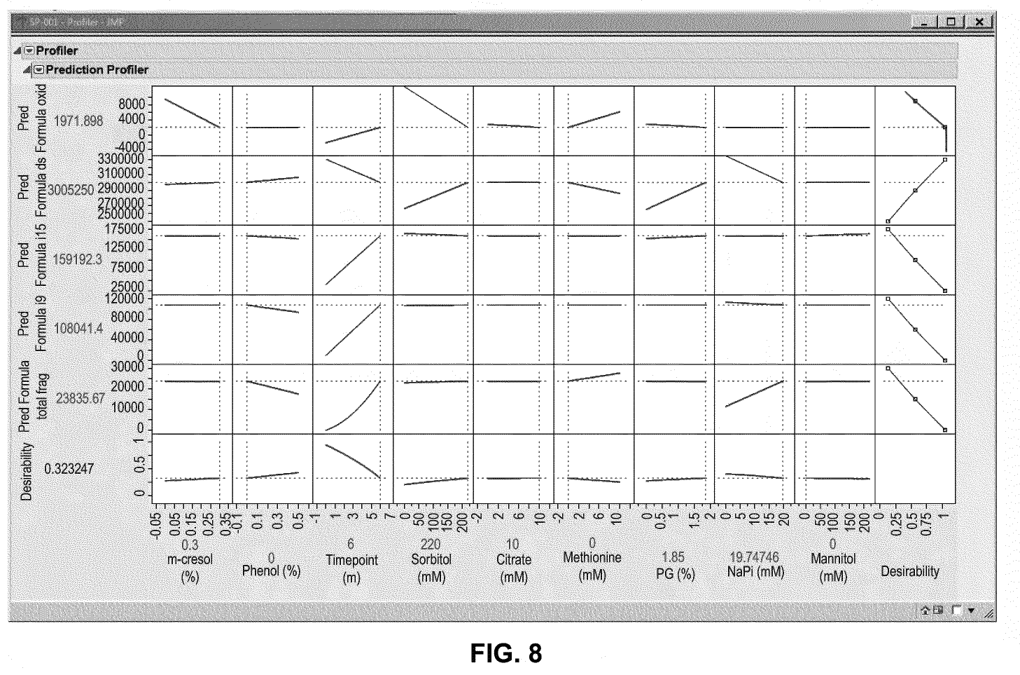

A prediction profiler model was used to estimate the impact of the various formulation components on total purity and impurities of seven MEDI0382 5 mg/mL formulations. The results are shown in FIG. 8. Mannitol and citrate had no impact on the purity profile. Sorbitol improves the total purity levels drug substance or “DS”) and decreases the level of oxidation. Methionine slightly increases the level of oxidation and decreases total impurities. Sodium phosphate decreases total purity and increases total impurity. M-cresol decreases levels of oxidation.

No major changes of osmolality, viscosity, or pH in the seven formulations were observed. Formulation G did not change pH over time even though it had no phosphate buffer. This suggests that sodium phosphate is not needed to control the pH in this formulation.

The visual inspection at 24 months showed a variation of appearance in cartridges containing air bubbles or not. Most of the cartridges containing air bubbles contained visible particles, whereas all of the cartridges without air bubbles did not contain visible particles. The exception was formulation G, where neither the cartridge containing air bubbles nor the cartridge without air bubbles contained visible particles.

Sub-visible particles were also monitored. Over the period of 24 months, formulation F was the only formulation in the cartridges stored at 5° C. that showed an increase in particles. Fiber-like particles were also visible in several of the formulations stored in vials at 25° C. at about 6 months. The size of the particles was about 5-100 um. The size of the fiber-like particles in the cartridge for formulation F at 24 months at 5° C. was smaller, 5-40 um. Fibers appeared to form more readily in vials than in cartridges, potentially as a result of the larger air interface in the vial.

Overall, formulations A-G had similar degradation profiles at rates of about 1.1-2% per year at 5° C. The degradation rates were not affected by the type of isotonicity agent (sorbitol, mannitol, or propylene glycol), but all formulations containing propylene glycol had high levels of aggregation as measured by DLS starting at 12 months when stored at 5° C. No gelation was observed over a period of 2 years at 5° C. or 6 months at 25° C. There was no measurable difference between sorbitol and mannitol as tonicity agents. All formulations containing sorbitol, as well as the mannitol formulation, showed physicochemical stability over 2 years. Sorbitol and m-cresol may improve total impurity levels. Therefore, sorbitol as a tonicity agent and a target pH of 8.1 appeared to provide the most stable formulation. Because it is unclear if lower concentrations of MEDI0382 would be stable in the absence of sodium phosphate buffer, sodium phosphate buffer was considered useful even though it may decrease total purity.

### Example 15: Preservative Efficacy

The efficacy of the preservatives in the seven MEDI0382 formulations evaluated in Example 14 was also assayed using the European Pharmacopeia Efficacy of Antimicrobial Preservation test. The results are shown in Table 10.

TABLE 10
Data from Preservative Efficacy Tests (PET) of Seven MEDI0382

| | | | Day | 6 | 24 | Day | Day | Day |
|---|---|---|---|---|---|---|---|---|
| | | Inoculum | 0 | hour | hour | 7 | 14 | 28 |
| Formulation | Organism | Log CFU/mL | | | | | | |
| Formulation | S. aureus | 5.8 | 5.7 | 5.5 | 4.9 | <1.0 | <1.0 | <1.0 |
| Buffer A | P. aerug | 6.0 | 5.9 | 1.6 | <1.0 | <1.0 | <1.0 | <1.0 |
| | E. coli | 5.9 | 5.9 | 5.1 | <1.0 | <1.0 | <1.0 | <1.0 |
| | C. albicans | 5.7 | 5.8 | 5.6 | 5.6 | 2.4 | <1.0 | <1.0 |
| | A. brasiliensis | 5.5 | 5.4 | 5.3 | 4.8 | <1.0 | <1.0 | <1.0 |
| Formulation | S. aureus | 5.8 | 5.8 | 5.0 | 5.6 | <1.0 | <1.0 | <1.0 |
| Buffer B | P. aerug | 6.0 | 5.9 | 2.5 | <1.0 | <1.0 | <1.0 | <1.0 |
| | E. coli | 5.9 | 5.9 | 5.5 | 2.3 | <1.0 | <1.0 | <1.0 |
| | C. albicans | 5.7 | 5.9 | 5.7 | 5.7 | 3.7 | <1.0 | <1.0 |
| | A. brasiliensis | 5.5 | 5.4 | 5.3 | 4.9 | <1.0 | <1.0 | <1.0 |
| Formulation | S. aureus | 5.8 | 5.6 | 5.7 | <1.0 | <1.0 | <1.0 | <1.0 |
| Buffer C | P. aerug | 6.0 | 4.6 | <1.0 | <1.0 | <1.0 | <1.0 | <1.0 |
| | E. coli | 5.9 | 5.9 | <1.0 | <1.0 | <1.0 | <1.0 | <1.0 |
| | C. albicans | 5.7 | 5.8 | 5.5 | 5.0 | <1.0 | <1.0 | <1.0 |
| | A. brasiliensis | 5.5 | 5.6 | 5.3 | 3.9 | <1.0 | <1.0 | <1.0 |
| Formulation | S. aureus | 5.6 | 5.8 | 5.3 | 1.9 | <1.0 | <1.0 | <1.0 |
| Buffer D | P. aerug | 5.8 | 5.8 | <1.0 | <1.0 | <1.0 | <1.0 | <1.0 |
| | E. coli | 6.0 | 6.0 | 4.9 | <1.0 | <1.0 | <1.0 | <1.0 |
| | C. albicans | 5.8 | 5.8 | 5.7 | 5.5 | <1.0 | <1.0 | <1.0 |
| | A. brasiliensis | 5.7 | 5.7 | 5.4 | 4.9 | <1.0 | <1.0 | <1.0 |
| Formulation | S. aureus | 5.6 | 5.9 | 5.2 | 1.3 | <1.0 | <1.0 | <1.0 |
| Buffer E | P. aerug | 5.8 | 5.8 | <1.0 | <1.0 | <1.0 | <1.0 | <1.0 |
| | E. coli | 6.0 | 6.0 | 3.9 | <1.0 | <1.0 | <1.0 | <1.0 |
| | C. albicans | 5.8 | 5.8 | <1.0 | <1.0 | <1.0 | <1.0 | <1.0 |
| | A. brasiliensis | 5.7 | 5.7 | 5.4 | 4.9 | <1.0 | <1.0 | <1.0 |
| Formulation | S. aureus | 5.6 | 5.7 | 4.9 | <1.0 | <1.0 | <1.0 | <1.0 |
| Buffer F | P. aerug | 5.8 | 5.8 | <1.0 | <1.0 | <1.0 | <1.0 | <1.0 |
| | E. coli | 6.0 | 5.6 | 1.3 | <1.0 | <1.0 | <1.0 | <1.0 |
| | C. albicans | 5.8 | 5.8 | 5.7 | 5.5 | <1.0 | <1.0 | <1.0 |
| | A. brasiliensis | 5.7 | 5.7 | 5.6 | 4.8 | <1.0 | <1.0 | <1.0 |

None of formulations A-G passed the strict (“A”) European criteria. This is in spite of the fact that the formulations contained concentrations of phenol or m-cresol that were close to the highest concentrations previously approved by the Food and Drug Administration (i.e., 0.55% w/v phenol in Victoza® and 0.315% w/v m-cresol in Humalog®). Typically Staphylococcus aureus and Escherichia coli failed at the 6 or 24-hours time point.

### Example 16: Formulation at 1-2 mg/mL MEDI0382

Clinical results demonstrated that less concentrated formulations of MEDI0382 would be desirable, so additional studies were performed to assess the long-term stability of 1 and 2 mg/mL MEDI0382 formulations and to further improve the antimicrobial activity of the formulation. The three formulations shown in Table 11 were tested in the long-term stability (LTS) study.

TABLE 11
1 and 2 mg/mL MEDI0382 Formulations Tested in LTS Assay

| | MEDI0382 | |
|---|---|---|
| Formulation | conc. | Buffer Composition |
| 1 | 2 mg/mL | 20 mM Sodium phosphate (NaPi), |
| | | 220 mM Sorbitol, 0.55% phenol, pH 8.1 |
| 2 | 1 mg/mL | 20 mM Sodium phosphate (NaPi), |
| | | 220 mM Sorbitol, 0.55% phenol, pH 8.1 |
| 3 | 1 mg/mL | 20 mM Sodium phosphate (NaPi), |
| | | 220 mM Sorbitol, 0.3% m-cresol, pH 8.1 |

The results of the long-term stability assay are shown in Table 12.

TABLE 12
Results of LTS Assay on 1 and 2 mg/mL MEDI0382 Formulations

| | | % Purity After | | % loss | |
|---|---|---|---|---|---|
| | Post- | 3 Months | | per month | |
| Formulation | Shipping | 5° C. | 25° C. | 5° C. | 25° C. |
| 1 | 98.1 | 97.9 | 92.7 | 0.1 | 1.8 |
| 2 | 97.8 | 97.8 | 91.8 | 0 | 2.0 |
| 3 | 96.8 | 96.8 | 91.1 | 0 | 2.0 |

Preservative efficacy testing (PET) was also performed on the same three formulations assaying the two microorganisms that caused failure in Example 15. The results are shown in Table 13.

TABLE 13
Results of PET on 1 and 2 mg/mL MEDI0382 Formulations

| | log red | | log red | |
|---|---|---|---|---|
| | T0 (bulk) | | T 1M (25 C.) cartridge | |
| Time | 6 h | 24 h | 6 h | 24 h |
| Buffer 1 | | | | |
| Staph | 0.4 | 4.2 | 0.3 | 3.2 |
| E coli | 4.7 | 5.1 | 4.6 | 5.0 |
| Buffer 2 | | | | |
| Staph | 1.0 | 5.2 | 0.7 | 5.4 |
| E coli | 5.1 | 5.1 | 5.0 | 5.0 |
| Buffer 3 | | | | |
| Staph | 0.5 | 3.4 | 0.3 | 2.8 |
| E coli | 5.1 | 5.1 | 5.0 | 5.0 |

All of the 1 and 2 mg/mL MEDI0382 formulations passed the less stringent European (“B”) criteria for Staphylococcus aureus (<2 log reductions 6 hours) and the stricter European (“A”) criteria for E. coli (>2 log reduction at 6 hours and >3 at 24 hours). There were no significant differences in the results between time zero and 25° C. stored in cartridge for a month.

### Example 17: Stability and Antimicrobial Activity of 2 mg/mL MEDI0382 Formulations

Various formulations containing 220 mM sorbitol, 20 mM sodium phosphate, sodium hydroxide to adjust the pH to 8.1, MEDI0382 (1 or 2 mg/mL) and either phenol (solid or liquid) or meta-cresol were further studied. The formulations tested in these experiments are shown in Table 14.

TABLE 14
MEDI0382 Formulations for Short-Term Stability Study

| Formulation | | | Preservative | Sorbitol | NaOH | Buffer | |
|---|---|---|---|---|---|---|---|
| number | Preservative | mg/mL | % w/v | mM | mM | mM | pH |
| 1 | Liquid Phenol | 1 | 0.35 | 220 | 1 | 20 | 8.1 |
| 2 | | | 0.45 | | | | |
| 3 | | | 0.55 | | | | |
| 4 | Phenol detached | | 0.35 | | | | |
| 5 | crystals | | 0.45 | | | | |
| 6 | | | 0.55 | | | | |
| 7 | Phenol detached | 2 | 0.35 | | 2 | | |
| 8 | crystals | | 0.45 | | | | |
| 9 | | | 0.55 | | | | |
| 10 | m-Cresol (Sigma) | 1 | 0.2 | | 1 | | |
| 11 | | | 0.25 | | | | |
| 12 | | | 0.3 | | | | |
| 13 | m-Cresol | 1 | 0.2 | | 1 | | |
| 14 | (Hedinger) | | 0.25 | | | | |
| 15 | | | 0.3 | | | | |

To make these formulations, sodium phosphate monobasic monohydrate (34 mg) and sodium phosphate dibasic heptahydrate (1.01 g) were dissolved in 80% fill (160 mL) of 1 or 2 mM NaOH (as listed in Table 14) for 20-30 minutes with magnetic stirring. To this solution, D-sorbitol (8.02 g) was added and mixed for 10 minutes. The additions of phenol, m-Cresol, and MEDI0382 were performed in a glove box. Depending on the preservative, a density and purity corrected weight was added. Solutions were then left to mix for 1 hour with the aid of magnetic stirring MEDI0382 was added with an excess of 10% to account for the purity and water content. Samples were then left to dissolve without agitation for 30 minutes at room temperature.

The pH was then determined and adjusted using 100 mM NaOH. Solutions were then brought to 200 mL using deionized milli-Q water. The peptide and preservative concentrations for each solution were determined using RP-UPLC where MEDI0382 was diluted to 0.5 mg/mL. Samples were then topped with either peptide or preservative to achieve the desired concentrations as listed in Table 14. The actual concentrations (as measured by RP-UPLC) as compared to the target concentrations are shown in Table 15.

TABLE 15
Target vs. Actual Preservative and Peptide Concentrations
in MEDI0382 Formulations

| | | | [Peptide] | [Peptide] |
|---|---|---|---|---|
| | Target | Actual | (mg/mL) | (mg/mL) |
| Preservative | (% w/v) | (% w/v) | target | actual |
| Liquid | 0.35 | 0.38 | 1 | 1.04 |
| phenol | 0.45 | 0.45 | 1 | 1.01 |
| | 0.55 | 0.56 | 1 | 1.07 |
| Solid Phenol | 0.35 | 0.35 | 1 | 1.11 |
| | 0.45 | 0.44 | 1 | 1.07 |
| | 0.55 | 0.57 | 1 | 1.13 |
| Solid Phenol | 0.35 | 0.38 | 2 | 2.14 |
| | 0.45 | 0.43 | 2 | 1.70 |
| | 0.55 | 0.54 | 2 | 2.00 |
| m-Cresol | 0.20 | 0.22 | 1 | 1.13 |
| (sigma) | 0.25 | 0.28 | 1 | 0.96 |
| | 0.30 | 0.30 | 1 | 1.01 |
| m-Cresol | 0.20 | 0.22 | 1 | 1.04 |
| (Hedinger) | 0.25 | 0.25 | 1 | 1.01 |
| | 0.30 | 0.31 | 1 | 1.05 |

The resulting solutions were protected from light, and 3 mL of each solution was aseptically filled into 3 cc vials (SCHOTT 10 cc 20 mm Falcon 10R; Part; CM1023) using 0.2 μm PVDF filters and 5 mL BD Plastik filters.

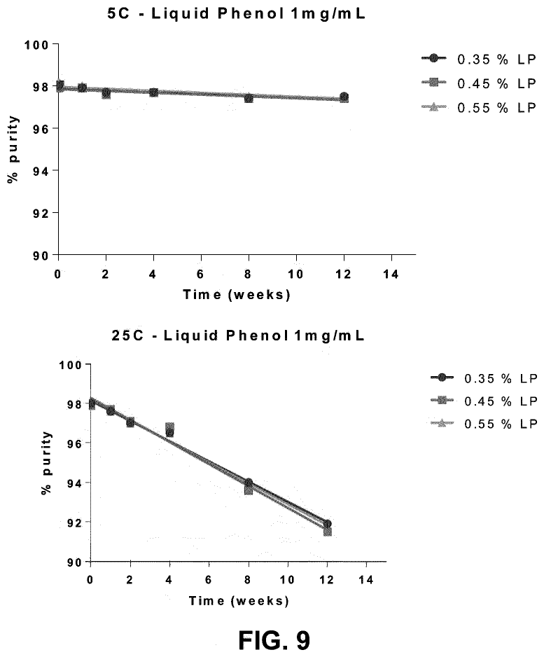

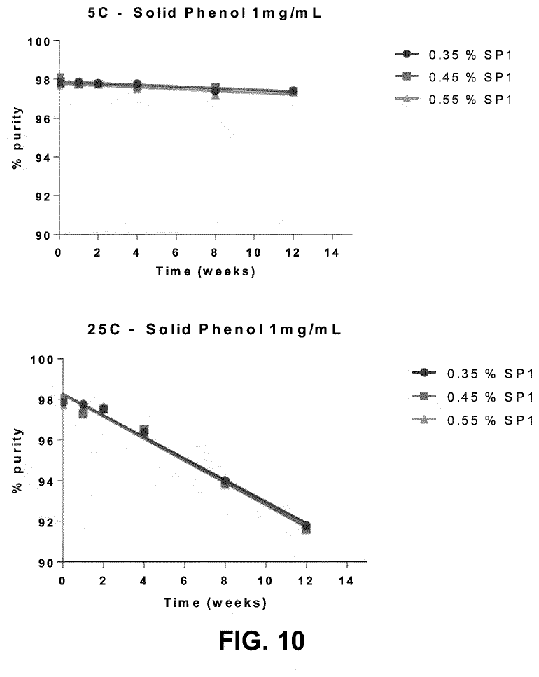

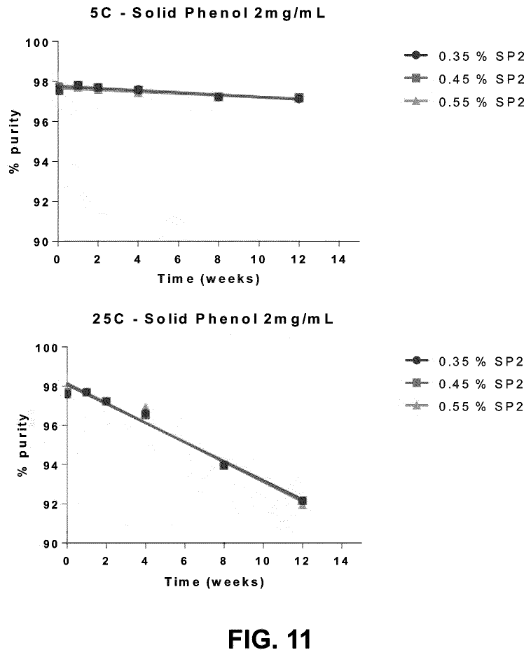

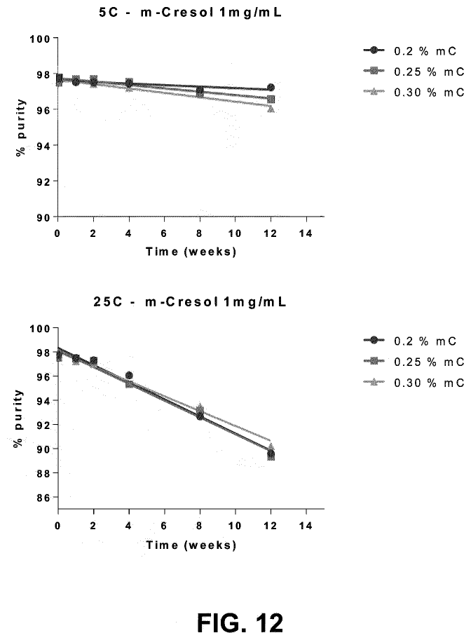

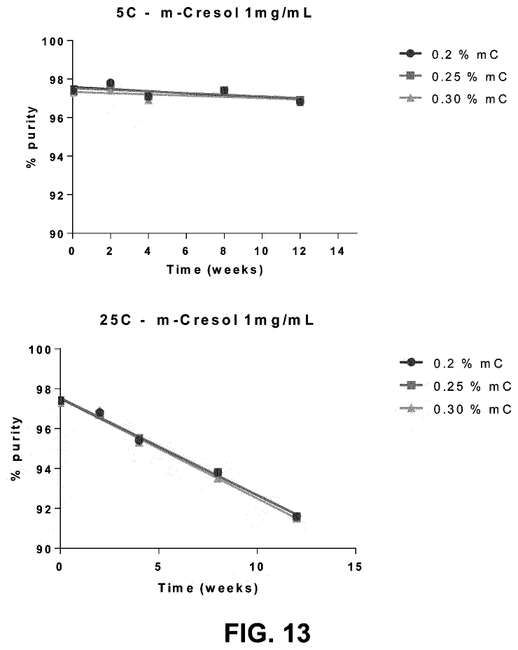

Each sample was tested for stability and preservative efficacy. The results of the chemical stability assays are shown in FIGS. 9-13. The results of the physical stability assays are shown in Table 16 below.

TABLE 16
Physical Stability of MEDI0382 Formulations for
Short-Term Stability Study

| | | | Rate of purity | |
|---|---|---|---|---|
| | | | loss/month | |
| | [Preservative] | [peptide] | (%) | |
| Preservative | % | mg/ml | 5 C. | 25 C. |
| Liquid phenol | 0.35 | 1 | 0.17 | 2.2 |
| | 0.35 | | 0.17 | 2.4 |
| | 0.55 | | 0.21 | 2.2 |
| Solid phenol | 0.35 | 1 | 0.17 | 2.2 |
| | 0.45 | | 0.17 | 2.6 |
| | 0.55 | | 0.21 | 2.2 |
| m-Cresol | 0.2 | 1 | 0.17 | 3.0 |
| (SIGMA) | 0.25 | | 0.42 | 2.9 |
| | 0.3 | | 0.50 | 2.6 |
| m-Cresol | 0.2 | 1 | 0.18 | 2.0 |
| (HEDINGER) | 0.25 | | 0.16 | 2.0 |
| | 0.3 | | 0.16 | 2.0 |
| Solid phenol | 0.35 | 2 | 0.21 | 2.1 |
| | 0.45 | | 0.21 | 2.1 |
| | 0.55 | | 0.21 | 2.1 |

In preservative efficacy assays, a short study was performed whereby two microorganisms were used to assess the microbial efficacy of the prepared formulations (Staphylococcus aureus and E. coli). Samples were tested at t=0 (bulk) and t=1 month after storage at 25° C. The results of these assays are shown in Table 17 below:

TABLE 17
PET Assessment of MEDI0382 Formulations for Short-Term
Stability Study

| | [Peptide] | | S.A. | | E. coli | |
|---|---|---|---|---|---|---|
| | (mg/mL) | Actual | | 1 month | | T 1 month |
| Preservative | actual | (% w/v) | T0 | 25° C. | T0 | 25° C. |
| Liquid phenol | 1.04 | 0.38 | Fail | Fail | B | B |
| | 1.01 | 0.45 | B | B | A | A |
| | 1.07 | 0.56 | B | B | A | A |
| Solid Phenol | 1.11 | 0.35 | Fail | Fail | B | B |
| | 1.07 | 0.44 | B | B | A | A |
| | 1.13 | 0.57 | B | B | A | A |
| Solid Phenol | 2.14 | 0.38 | Fail | Fail | B | B |
| | 1.70 | 0.43 | Fail | Fail | B | B |
| | 2.00 | 0.54 | B | B | A | A |
| m-Cresol | 1.13 | 0.22 | Fail | Fail | B | B |
| (sigma) | 0.96 | 0.28 | B | B | A | A |
| | 1.01 | 0.30 | B | B | A | A |
| m-Cresol | 1.04 | 0.22 | B | B | A | A |
| (Hedinger) | 1.01 | 0.25 | B | B | A | A |
| | 1.05 | 0.31 | A | A | A | A |

With regard to stability, these studies demonstrated that the concentration of either preservative did not significantly affect the peptide chemical or physical stability within the ranges tested. In addition, the peptide concentration (of either 1 or 2 mg/mL) did not affect the rates of degradation, when solid phenol was used as the preservative. The type of preservative type did show some effect on MEDI0382 chemical degradation rates. Hedinger m-Cresol appeared to slow the rates of chemical degradation as compared to the rates observed using Sigma m-Cresol.

With regard to antimicrobial activity, these studies demonstrated that all preservatives tested passed the less stringent European (“B”) criteria the highest concentrations tested. However, Hedinger m-Cresol passed the more stringent European (“A”) criteria when the highest concentration was used. In order to achieve the less stringent European (“B”) criteria, at least 0.44% w/v of phenol is needed with 1 mg/mL MEDI0382 or at least 0.54% w/v of phenol is needed with 2 mg/mL MEDI0382. None of the formulations containing phenol up to 0.56% w/v passed the more stringent European (“A”) criteria. The preservative efficacy was retained after 1 month storage at 25° C.

Based on these results, formulation 3 (220 mM sorbitol, 20 mM sodium phosphate, 1 mg/mL MEDI0382, 0.3% w/v meta-cresol, and NaOH to adjust to pH 8.1) appeared to be the most favorable formulation.

### Example 18: Selection of Meta-Cresol Concentration

Additional experiments were performed to identify the optimum concentration of meta-cresol to be used in a formulation based on formulation 3 in Example 17 above. As shown in Table 18, the concentration of m-cresol was varied in formulation 3, and the microbial efficacy against Staphylococcus aureus and other bacteria was examined.

TABLE 18
Effect of M-Cresol Concentration on Microbial Efficacy

| | | Log reduction | Log reduction |
|---|---|---|---|
| Meta-cresol | Compliance with Ph. | in S. aureus | in S. aureus |
| (w/v %) | Eur. | at 6 hours | at 24 hours |
| 0 | | <0.1 | 1.0 |
| 0.17 | Fails for S. aureus only | 0.2 | 0.3 |
| | on Ph. Eur. Criteria B, | | |
| | other bacteria pass Ph. | | |
| | Eur. criteria B | | |
| 0.24 | Fails for S. aureus only | 0.2 | 0.3 |
| | on Ph. Eur. Criteria B, | | |
| | other bacteria pass Ph. | | |
| | Eur. criteria A | | |
| 0.27 | Pass Criteria B for S. | 1.0 | 3.0 |
| | aureus, other bacteria | | |
| | pass Ph. Eur. criteria A | | |
| 0.28 | Pass Criteria B for S. | 1.0 | 2.4 |
| | aureus, other bacteria | | |
| | pass Ph. Eur. criteria A | | |
| 0.34 | Pass Ph. Eur. criteria A | 2.1 | >5.1 |
| 0.42 | Pass Ph. Eur. criteria A | >5.1 | >5.1 |

In addition, complementary studies at target m-cresol concentration 0.31% w/v were performed to evaluate assay variability. These studies are summarized in Table 19.

TABLE 19
Variability in Microbial Activity Assay

| | MEDI0382 | | Log reduction | Log reduction |
|---|---|---|---|---|
| Meta-cresol | Conc | Compliance with Ph. | in S. aureus | in S. aureus |
| (w/v %) | (mg/mL) | Eur. | at 6 hours | at 24 hours |
| 0.30 | 0.9 | Pass Ph. Eur. criteria A | 2.2 | 5.1 |
| 0.29 | 0.9 | Pass Ph. Eur. criteria A | 2.4 | >5.1 |
| 0.31 | 0.9 | Pass Criteria B for S. | 1.7 | >5.1 |
| | | aureus, other bacteria | | |
| | | pass Ph. Eur. criteria A | | |
| 0.30 | 1 | Pass Criteria B for S. | 0.4 | >4.9 |
| | | aureus, other bacteria | | |
| | | pass Ph. Eur. criteria A | | |
| 0.33 | 1 | Pass Criteria B for S. | 1.9 | >4.9 |
| | | aureus, other bacteria | | |
| | | pass Ph. Eur. criteria A | | |
| 0.29 | 1 | Pass Criteria B for S. | 1.4 | >4.9 |
| | | aureus, other bacteria | | |
| | | pass Ph. Eur. criteria A | | |

These results demonstrate that the effectiveness of the preservative is dependent on the concentration of MEDI0382 and shows some variability. In addition, in order to consistently achieve Ph. Eur. Criteria B, a minimum of 0.27% (w/v) of meta-cresol is needed, and to achieve Ph. Eur. Criteria A, a minimum of 0.34% (w/v) of meta-cresol is needed.

Taking this into account, a m-cresol concentration of 0.31% w/v (+/−10%) appears to be favorable. This concentration shows an appropriate leave of efficacy for a once daily product, reducing all bacteria by 3 log after 24 hours.

A recipe for producing the highly advantageous formulation containing 220 mM sorbitol, 20 mM sodium phosphate, 0.31% (w/v) meta-cresol, and NaOH to adjust to pH 8.1 is shown in Table 20. The 1 mg MEDI0382 formulation is the titration dose product, and the 5 mg MEDI0382 formulation is the maintenance dose product.

TABLE 20
Recipe for MEDI0382 Formulation

| | Amount | mM |
|---|---|---|
| Item description | per mb | concentration |
| MEDI0382 | 1 mg, 2 mg, | |
| | or 5 mg | |
| Sodium phosphate monobasic | 0.13 mg | 1.0 |
| monohydrate | | |
| Sodium phosphate dibasic heptahydrate | 5.12 mg | 19.0 |
| Sorbitol | 40.13 mg | 220.3 |
| meta-Cresol | 3.10 mg | 28.6 |
| Sodium hydroxide-for injection | q.s. pH 8.1 | |
| Water for injection (WFI) | 966.5 mg | |

### Example 19: Impact of Sodium Phosphate Concentration and Salt Type on High Molecular Weight Impurities

Covalent dimers were identified as impurities in MEDI0382 multi-dose formulations. Therefore, the impact of sodium phosphate on formation of high molecular weight (HWM) impurities on MEDI0382 was examined.

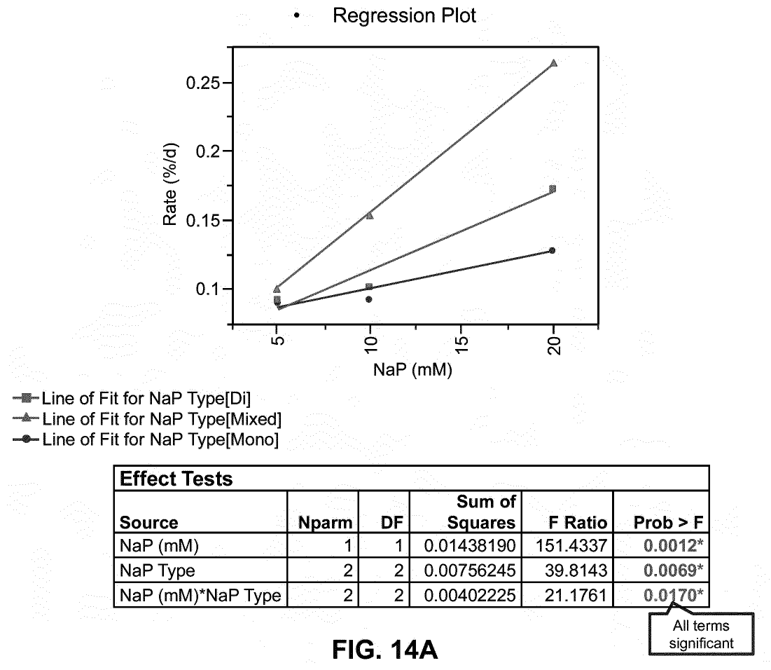

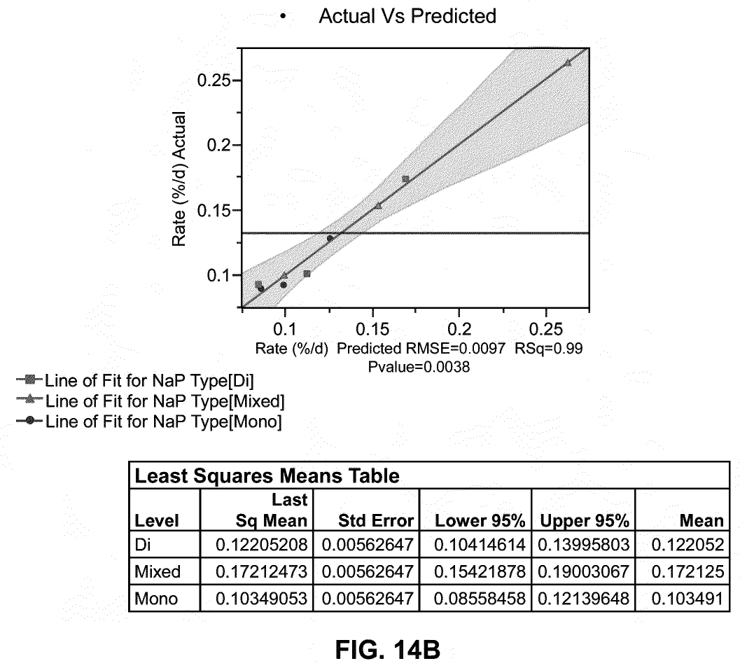

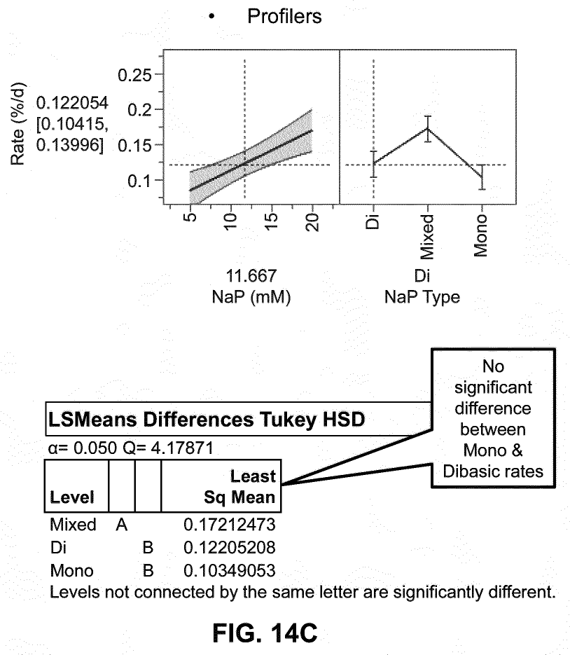

Formulations containing m-cresol 0.31% (w/v), sorbitol 220 mM, and MEDI0382 (1 mg/mL) were prepared with varying amounts (0 to 20 mM) of sodium phosphate monobasic and dibasic salt, only dibasic salt, and only mono basic salt. Samples were placed at 5° C. and 25° C. and analyzed for stability by SEC, RP UPLC and LC-MS. The results, which are shown in FIGS. 14A, 14B, and 14C, demonstrate that sodium phosphate concentration has a significant impact on rates of HMW impurities at the tested temperatures. The LC MS impurity identification shows that the formulation without sodium phosphate reduced the major HMW impurity found in the formulation.

These data indicate that lower sodium phosphate concentrations improve stability. Therefore an alternative MEDI0382 formulation with less salt was developed, as well as an alternative formulation with a low concentration of TRIS base (tromethamine) as a replacement of sodium phosphate. Recipes for the alternative formulations are shown in the following Tables 21 to 25.

TABLE 21
Recipe for Alternative MEDI0382 Formulation 1

| | Amount | mM |
|---|---|---|
| Item description | per mb | concentration |
| MEDI0382 | 1, 2, or 5 mg | |
| Sodium phosphate dibasic heptahydrate | 2.68 mg | 10 |
| Sorbitol | 40.13 mg | 220.3 |
| m-Cresol | 3.10 mg | 28.6 |
| WFI | 966.52 | |
| Sodium hydroxide | q.s. pH 8.1 | |

TABLE 22
Recipe for Alternative MEDI0382 Formulation 2

| | Amount | mM |
|---|---|---|
| Item description | per mL | concentration |
| MEDI0382 | 1 mg or 5 mg | |
| Sodium phosphate monobasic | 0.06 mg | 0.5 |
| monohydrate | | |
| Sodium phosphate dibasic heptahydrate | 2.56 mg | 9 |
| Sorbitol | 40.13 mg | 220.3 |
| meta-Cresol | 3.10 mg | 28.6 |
| Sodium hydroxide-for injection | q.s. pH 8.1 | |
| Water for injection (WFI) | 966.5 mg | |

TABLE 23
Recipe for Alternative MEDI0382 Formulation 3

| | Amount | mM |
|---|---|---|
| Item description | per mL | concentration |
| MEDI0382 | 1, 5 mg | |
| Sodium phosphate dibasic heptahydrate | 1.34 mg | 5 |
| Sorbitol | 40.13 mg | 220.3 |
| m-Cresol | 3.10 mg | 28.6 |
| WFI | 966.52 | |
| Sodium hydroxide | q.s. pH 8.1 | |

TABLE 24
Recipe for Alternative MEDI0382 Formulation 4

| | Amount | mM |
|---|---|---|
| Item description | per mb | concentration |
| MEDI0382 | 5 mg | |
| TRIS base (tromethamine) | 1.21 mg | 10 |
| Sorbitol | 40.13 mg | 220.3 |
| m-Cresol | 3.10 mg | 28.6 |
| WFI | q.s. 1 mL | |
| Sodium hydroxide | q.s. pH 8.1 | |

TABLE 25
Recipe for Alternative MEDI0382 Formulation 5

| Item description | Amount per mL | | mM concentration |
|---|---|---|---|
| MEDI0382 | 1 | mg | |
| TRIS base (tromethamine) | 1.21 | mg | 10 |
| Sorbitol | 40.13 | mg | 220.3 |
| m-Cresol | 3.10 | mg | 28.6 |
| WFI | q.s. 1 | mL | |
| Hydrochloric acid | q.s. pH | 8.1 | |

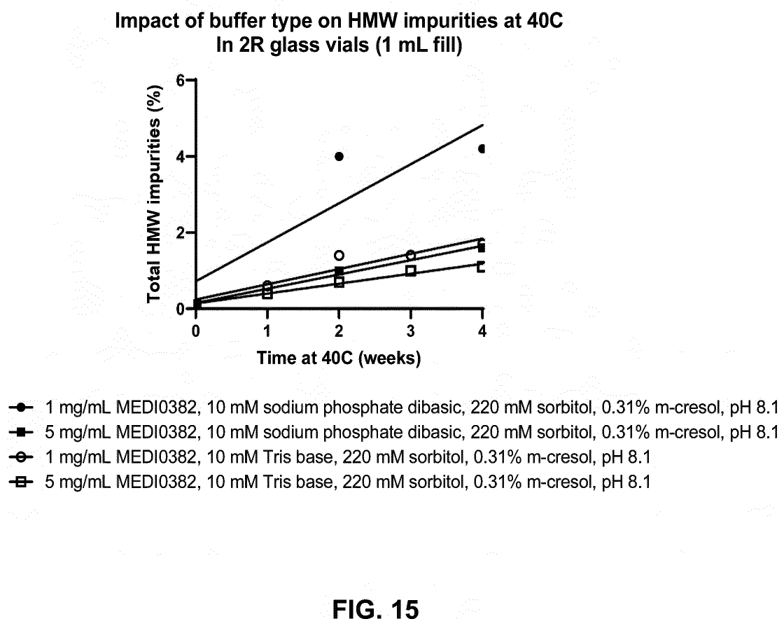

A stability study comparing TRIS base with sodium phosphate dibasic at MEDI0382 concentrations of 1 and 5 mg/mL (Formulations 4 and 5 versus Formulation 1) was run in glass vials. SEC data shows that replacing sodium phosphate dibasic by TRIS base allows reducing the level of HMW impurities (FIG. 15 and Table 26).

TABLE 26
Effect of Buffer Type on HMW Impurities
Levels at 2-8° C. and 25° C. (SEC Results)

| | Total HMW impurities (%) | | |
|---|---|---|---|
| | | After 1 | After 6 |
| | | month at | months at |
| Formulation composition | T0 | 2-8° C. | 25° C. |
| 1 mg/mL MEDI0382, 10 mM TRIS base, | 0.1 | 0.2 | 5.9 |
| 220 mM sorbitol, 0.31% m-cresol, pH 8.1 | | | |
| 5 mg/mL MEDI0382, 10 mM TRIS base, | 0.1 | 0.1 | 2.3 |
| 220 mM sorbitol, 0.31% m-cresol, pH 8.1 | | | |
| 1 mg/mL MEDI0382, 10 mM sodium | 0.1 | 0.4 | 9.4 |
| phosphate dibasic, 220 mM sorbitol, | | | |
| 0.31% m-cresol, pH 8.1 | | | |
| 5 mg/mL MEDI0382, 10 mM sodium | 0.1 | 0.2 | 2.7 |
| phosphate dibasic, 220 mM sorbitol, | | | |
| 0.31% m-cresol, pH 8.1 | | | |

### Example 20: Impact of 5 mg/ml Concentration of MEDI0382

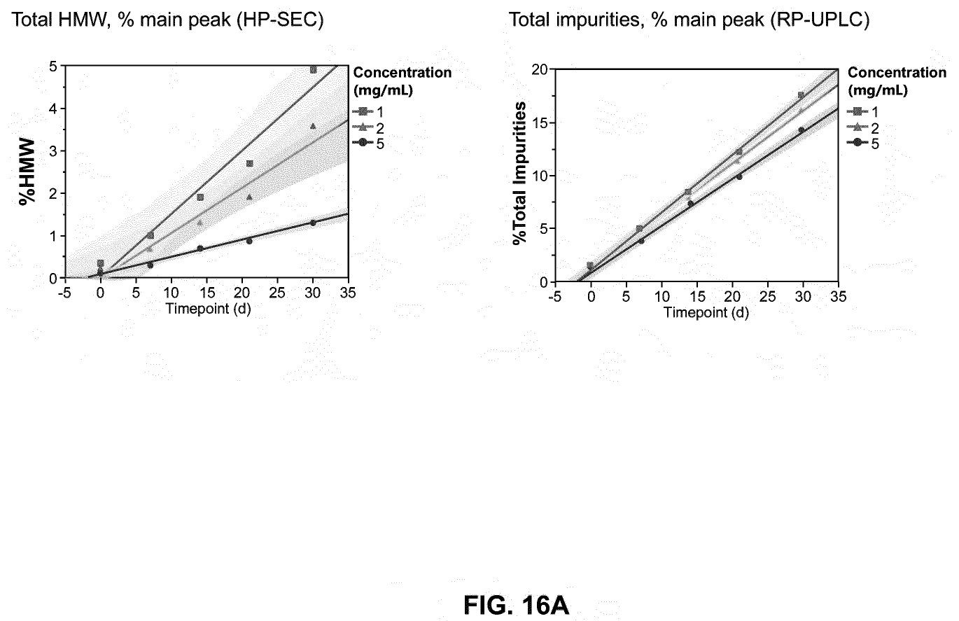

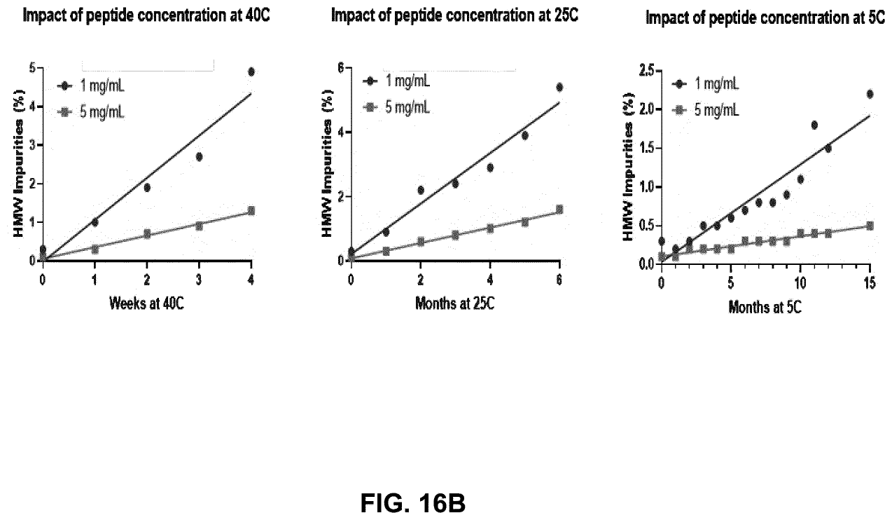

The 1, 2, and 5 mg/ml MEDI0382 formulations described in Table 20 were tested for HMW species and total impurities using methods essentially as described above. The results are shown in FIG. 16A and FIG. 16B. There was no significant difference in the amount of HMW species or total impurities between the 1 and 2 mg/ml formulations. However, the HMW species and total impurities were consistently lower in the 5 mg/ml formulations.

The stability of the three formulations was also measured in terms of peptide content and m-Cresol content over 30 days. The results are shown in Tables 27-34.

TABLE 27
Effect of MEDI0382 on Peptide Content
Peptide Content - Stability at 40° C. (D) mg/mL

| | 0 days | 7 days | 14 days | 21 days | 30 days |
|---|---|---|---|---|---|
| 1 mg/ml | 1.02 | 1.00 | 0.99 | 0.96 | 1.00 |
| 2 mg/ml | 1.96 | 1.95 | 1.92 | 1.88 | 2.00 |
| 5 mg/ml | 5.07 | 5.04 | 4.98 | 4.92 | 4.96 |

TABLE 28
Effect of MEDI0382 on m-Cresol Content
m-Cresol Content - Stability at 40° C. (D) w/v

| | Formulated | | | | | |
|---|---|---|---|---|---|---|
| | Bulk | | | | | |
| | Solution | | | | | |
| | (FBS) | 0 days | 7 days | 14 days | 21 days | 30 days |
| 1 mg/ml | 0.30 | 0.29 | 0.30 | 0.29 | 0.29 | 0.29 |
| 2 mg/ml | 0.30 | 0.29 | 0.30 | 0.28 | 0.28 | 0.29 |
| 5 mg/ml | 0.30 | 0.29 | 0.31 | 0.28 | 0.28 | 0.29 |

TABLE 29
MEDI0382 Content over Time at 40° C.
Peptide Content (mg/mL) - Stability at 40° C.

| | 0 days | 7 days | 14 days | 21 days | 30 days |
|---|---|---|---|---|---|
| 1 mg/ml | 1.02 | 1.00 | 0.99 | 0.96 | 1.00 |
| 5 mg/ml | 5.07 | 5.04 | 4.98 | 4.92 | 4.96 |

TABLE 30
MEDI0382 Content over Time at 25° C.
Peptide Content (mg/mL) - Stability at 25° C.

| | 0 months | 1 month | 3 months | 6 months |
|---|---|---|---|---|
| 1 mg/ml | 1.02 | 1.01 | 1.01 | 1.01 |
| 5 mg/ml | 5.07 | 4.94 | 4.98 | 4.98 |

TABLE 31
MEDI0382 Content over Time at 5° C.
Peptide Content (mg/mL) - Stability at 5° C.

| | | | | | | 12 |
|---|---|---|---|---|---|---|
| | 0 months | 1 month | 3 months | 6 months | 9 months | months |
| l mg/ml | 1.02 | 1.02 | 1.02 | 1.02 | 1.04 | 1.02 |
| 5 mg/ml | 5.07 | 4.92 | 5.01 | 5.06 | 4.90 | 5.16 |

TABLE 32
m-Cresol content over Time at 40° C.
m-Cresol Content (% w/v) - Stability at 40° C.

| | 0 days | 7 days | 14 days | 21 days | 30 days |
|---|---|---|---|---|---|
| 1 mg/ml | 0.29 | 0.30 | 0.29 | 0.29 | 0.29 |
| 5 mg/ml | 0.29 | 0.31 | 0.28 | 0.28 | 0.29 |

TABLE 33
m-Cresol content over Time at 25° C.
m-Cresol Content (% w/v) - Stability at 25° C.

| | 0 months | 1 month | 3 months | 6 months |
|---|---|---|---|---|
| 1 mg/ml | 0.29 | 0.29 | 0.29 | 0.28 |
| 5 mg/ml | 0.28 | 0.29 | 0.29 | 0.28 |

TABLE 34
m-Cresol content over Time at 5° C.
m-Cresol Content (% w/v) - Stability at 5° C.

| | | | | | | 12 |
|---|---|---|---|---|---|---|
| | 0 months | 1 month | 3 months | 6 months | 9 months | months |
| 1 mg/ml | 0.29 | 0.29 | 0.29 | 0.29 | 0.29 | 0.28 |
| 5 mg/ml | 0.28 | 0.29 | 0.29 | 0.29 | 0.29 | 0.28 |

These data demonstrate that as compared to the starting point, there were no significant differences in the three formulations for peptide content or m-Cresol content.

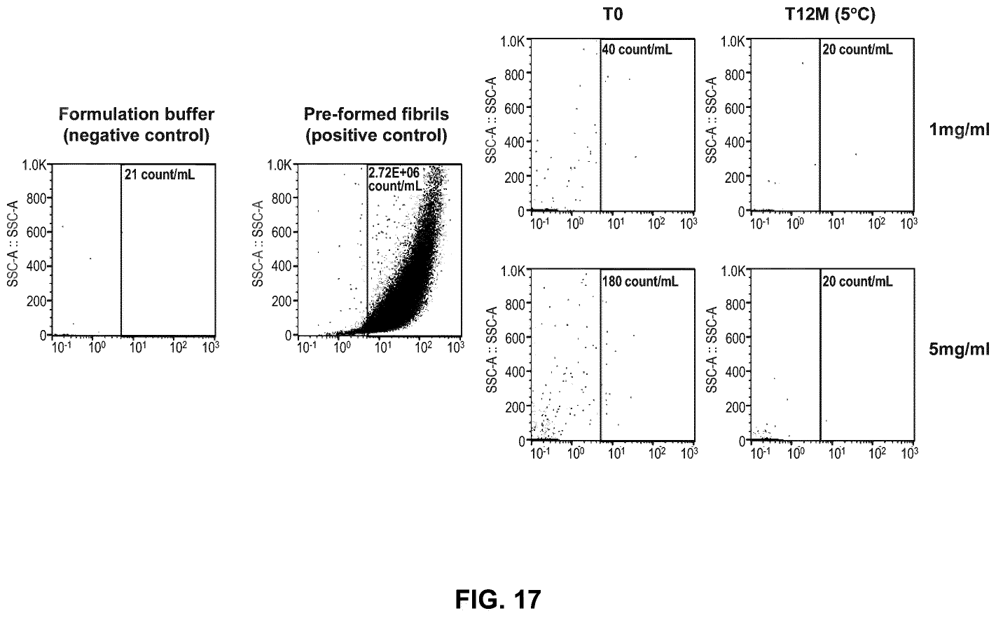

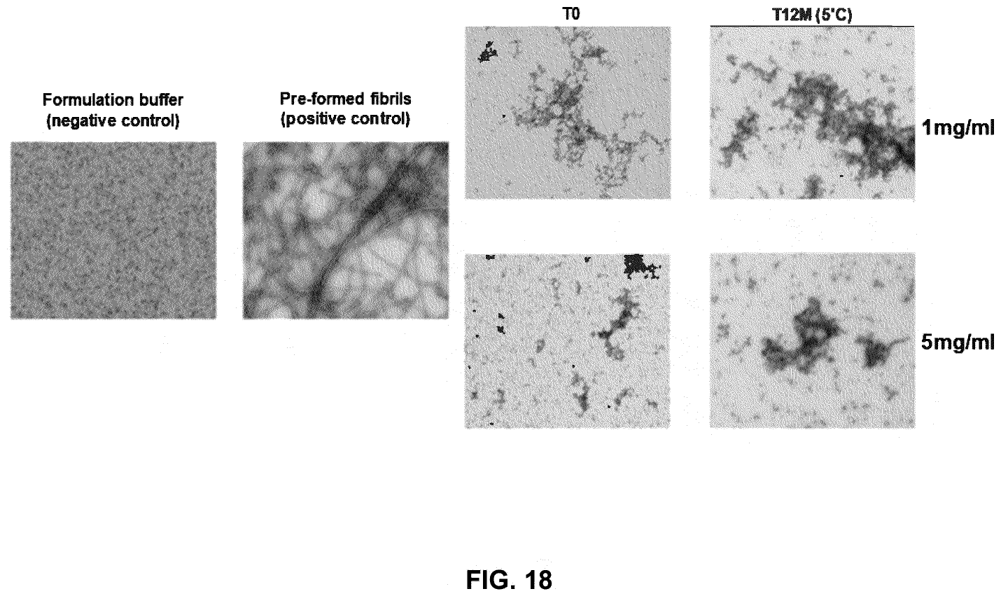

The formulations were also assessed for fibrillation positive particles by Fluorescence Activated Cell Sorting (FACS) in presence of ThT dye (see FIG. 17) and transmission electron microscopy (TEM) (FIG. 18). Comparison of the 1 & 5 mg/ml samples with the pre-formed fibrils (positive control) as well as the formulation buffer (negative control) indicates that no fibrillation-positive particles could be detected at any time point under any condition tested.

Preservative efficacy testing (PET) was also performed on 2 and 5 mg/ml MEDI0382 formulations containing 20 mM sodium phosphate buffer, 220 mM sorbitol, and m-Cresol (at the concentrations indicated in Table 35) at pH. 8.2

TABLE 35
PET results

| | MEDI0382 | m-Cresol | Results |
|---|---|---|---|
| Organism | (mg/mL) | (% w/v) | (EU) |
| S. aureus | 2.0 | 0.28 | Pass B |
| S. aureus | 2.0 | 0.31 | Pass B |
| S. aureus | 2.0 | 0.34 | Pass B |
| S. aureus | 5.0 | 0.28 | Pass B |
| S. aureus | 5.0 | 0.31 | Pass B |
| S. aureus | 5.0 | 0.34 | Pass A |

From the PET feasibility study, all 2 and 5 mg/mL MEDI0382 formulations concentrations with 0.28-0.34% w/v m-Cresol passed the European (“B”) criteria for Staphylococcus aureus.

### Example 21: Impact of MEDI0382 Concentration (1 vs 5 mg/ml) on the Stability of Sodium Dibasic-Based Formulation

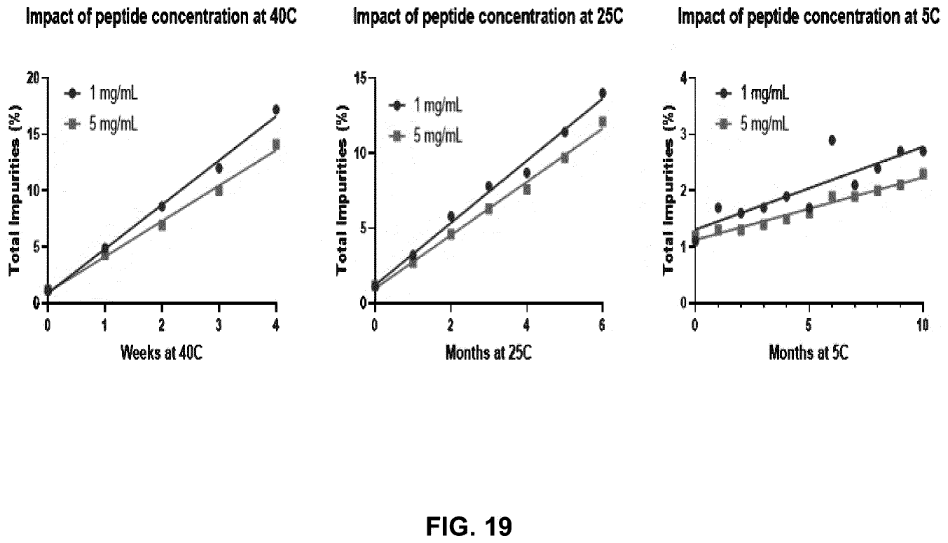

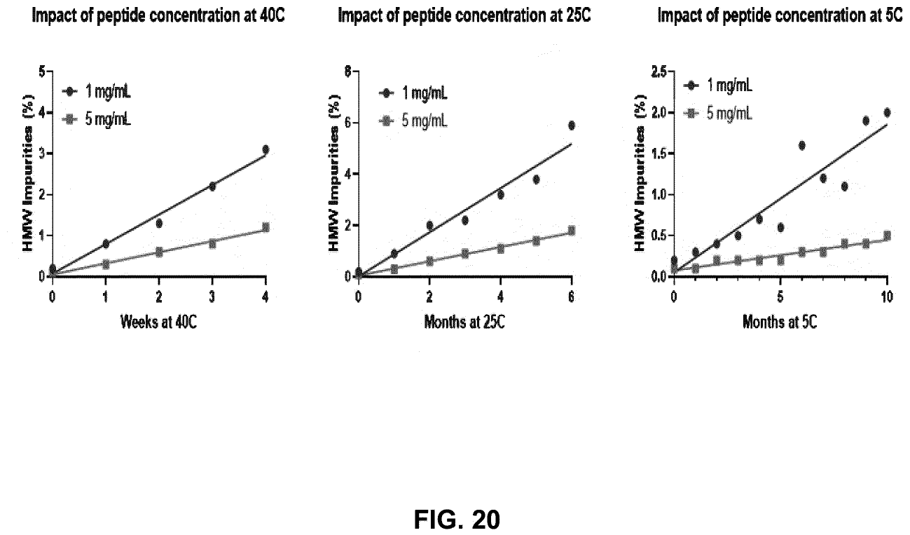

The stabilities of the 1 and 5 mg/ml MEDI0382 formulations as described in Table 21 (Alternative MEDI0382 Formulation 1) were assessed by means of HMW species and total impurities testing, using the methods as described above. The results are shown in FIG. 19 and FIG. 20. There was a marked reduction of total and HMW impurities for the 5 mg/ml formulation, as compared to the 1 mg/ml formulation, with the effect being more pronounced in case of the HMW impurities.

The stability of the two formulations was also assessed in terms of peptide content and m-Cresol content over 30 days at 5, 25 & 40° C. The results for the peptide content are shown in Tables 36-38 and for the m-cresol content in Tables 39-41.

TABLE 36
MEDI0382 Content over Time at 40° C.
Peptide Content (mg/mL) - Stability at 40° C.

| | 0 days | 7 days | 28 days |
|---|---|---|---|
| 1 mg/ml | 1.00 | 0.99 | 0.99 |
| 5 mg/ml | 5.07 | 5.01 | 5.02 |

TABLE 37
MEDI0382 Content over Time at 25° C.
Peptide Content (mg/mL) - Stability at 25° C.

| | 0 months | 1 month | 3 months | 6 months |
|---|---|---|---|---|
| 1 mg/ml | 1.00 | 1.00 | 0.98 | 0.99 |
| 5 mg/ml | 5.07 | 5.10 | 4.95 | 5.09 |

TABLE 38
MEDI0382 Content over Time at 5° C.
Peptide Content (mg/mL) - Stability at 5° C.

| | 0 months | 3 months | 6 months | 9 months |
|---|---|---|---|---|
| 1 mg/ml | 1.00 | 0.99 | 1.01 | 0.99 |
| 5 mg/ml | 5.07 | 5.01 | 5.11 | 4.88 |

TABLE 39
m-Cresol content over Time at 40° C.
m-Cresol Content (% w/v) - Stability at 40° C.

| | 0 days | 7 days | 28 days |
|---|---|---|---|
| 1 mg/ml | 0.31 | 0.31 | 0.30 |
| 5 mg/ml | 0.31 | 0.31 | 0.30 |

TABLE 40
m-Cresol content over Time at 25° C.
m-Cresol Content (% w/v) - Stability at 25° C.

| | 0 months | 1 month | 3 months | 6 months |
|---|---|---|---|---|
| 1 mg/ml | 0.31 | 0.30 | 0.30 | 0.30 |
| 5 mg/ml | 0.31 | 0.30 | 0.30 | 0.30 |

TABLE 41
m-Cresol content over Time at 5° C.
m-Cresol Content (% w/v) - Stability at 5° C.

| | 0 months | 3 months | 6 months | 9 months |
|---|---|---|---|---|
| 1 mg/ml | 0.31 | 0.30 | 0.30 | 0.28 |
| 5 mg/ml | 0.31 | 0.30 | 0.30 | 0.29 |

These data suggest that as compared to the starting point, there were no significant fluctuations in the peptide or m-Cresol content over time for both 1 and 5 mg/mL formulations.

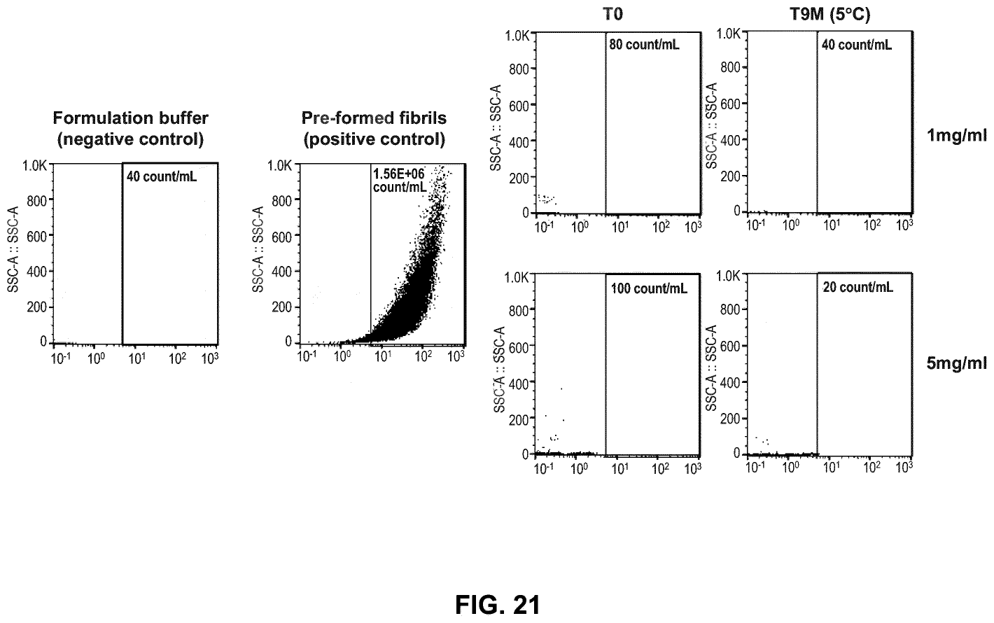

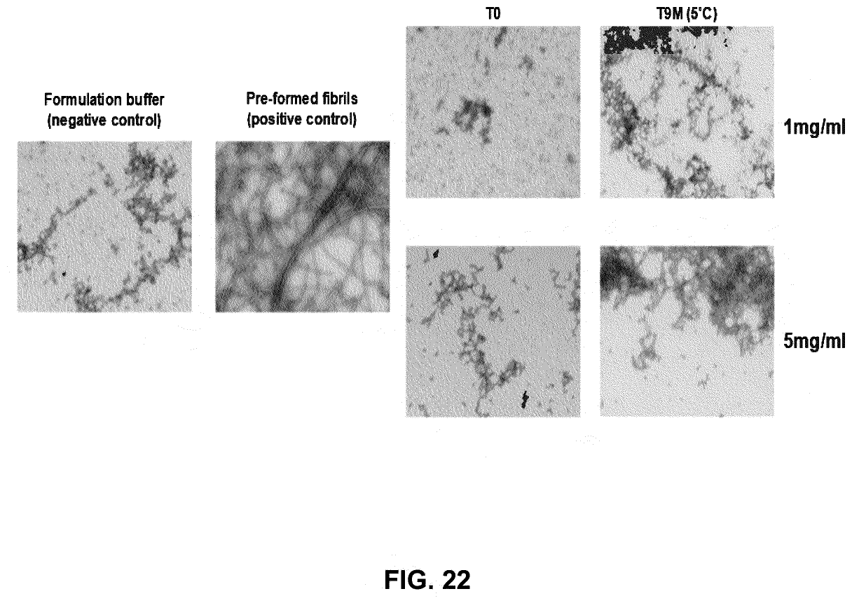

The formulations were also assessed for fibrillation positive particles by FACS (see FIG. 21) and transmission electron microscopy (TEM) (FIG. 22). Comparison of the 1 & 5 mg/ml samples with the pre-formed fibrils (positive control) as well as the formulation buffer (negative control) indicates that no fibrillation-positive particles could be detected at any of the time points or under any conditions tested.

### Example 22: Preservative Efficacy Robustness of 5 mg/mL MEDI0382 Multi-Dose Formulation

A multivariate robustness study was carried out to investigate the impact of m-cresol content, sodium phosphate concentration, and m-cresol content on the preservative efficacy of the 5 mg/mL multi-dose formulation. Previous studies at 1, 2, and 5 mg/mL showed that peptide concentration had a negative correlation with preservative efficacy, so this robustness study was carried out at a set concentration of 5.5 mg/mL for all formulations. A total of 20 bulk formulations were prepared and tested in accordance with the European Pharmacopeia Edition 10.0 Section 5.1.3 and the United States Pharmacopeia 42 <51>.

The data in Table 42 shows that at m-cresol concentrations of 0.24% (w/v) and above, USP and EP criteria A are consistently met for all microorganisms except Staphylococcus aureus, for which EP criteria B is met. For this microorganism, m-cresol concentrations of 0.28% (w/v) and above are required to achieve a log reduction consistently greater than 3 at 24 hours. Statistical analysis of the data showed that m-cresol had the highest impact on preservative efficacy, and pH and sodium phosphate concentration have a milder impact. This study confirmed that the high concentration multi-dose formulation has adequate antimicrobial properties for a once-daily administration.

TABLE 42
Preservative Efficacy Results of MEDI0382 High Concentration Multi-
Dose Formulation

| Formulation composition - | | | | | | | | |
|---|---|---|---|---|---|---|---|---|
| 5.5 mg/mL MEDI0382, 220 | | | | | | | | |
| mM sorbitol | | | | | | | | |
| | Sodium | | | Log reduction of colony forming units per | | | | |
| | phosphate | m-cresol | | mL of sample | | | | |
| | concentration | content | | 6 | 24 | | 14 | 28 |
| pH | (mM) | (% w/v) | Organism | hours | hours | 7 days | days | days |
| 7.8 | 25 | 0.23 | Pseudomonas aeruginosa | >4.94 | >4.94 | >4.94 | >4.94 | >4.94 |
| | | | Staphylococcus aureus | 0.48 | 0.89 | 3.48 | >4.89 | >4.89 |
| | | | Escherichia coli | NT¹ | NT | >4.98 | >4.98 | >4.98 |
| | | | Aspergillus hrasiliensis | NT | NT | >4.85 | >4.85 | >4.85 |
| | | | Candida albicans EP | NT | NT | 1.81 | >4.30 | >4.30 |
| | | | Candida albicans USP | NT | NT | 1.81 | >4.30 | >4.30 |
| 7.8 | 17.5 | 0.23 | Pseudomonas aeruginosa | >4.87 | >4.87 | >4.87 | >4.87 | >4.87 |
| | | | Staphylococcus aureus | 0.27 | 0.97 | >4.87 | >4.87 | >4.87 |
| | | | Escherichia coli | NT | NT | >4.76 | >4.76 | >4.76 |
| | | | Aspergillus hrasiliensis | NT | NT | >4.53 | >4.53 | >4.53 |
| | | | Candida albicans EP | NT | NT | 2.14 | >4.40 | >4.40 |
| | | | Candida albicans USP | NT | NT | 2.14 | >4.40 | >4.40 |
| 8.4 | 10 | 0.23 | Pseudomonas aeruginosa | >4.79 | >4.79 | >4.79 | >4.79 | >4.79 |
| | | | Staphylococcus aureus | 0.32 | 0.80 | >4.88 | >4.88 | >4.88 |
| | | | Escherichia coli | NT | NT | >4.70 | >4.70 | >4.70 |
| | | | Aspergillus hrasiliensis | NT | NT | >4.59 | >4.59 | >4.59 |
| | | | Candida albicans EP | NT | NT | >4.51 | >4.51 | >4.51 |
| | | | Candida albicans USP | NT | NT | >4.51 | >4.51 | >4.51 |
| 8.4 | 25 | 0.24 | Pseudomonas aeruginosa | >4.72 | >4.72 | >4.72 | >4.72 | >4.72 |
| | | | Staphylococcus aureus | 1.41 | >4.64 | >4.64 | >4.64 | >4.64 |
| | | | Escherichia coli | NT | NT | >4.59 | >4.59 | >4.59 |
| | | | Aspergillus hrasiliensis | NT | NT | >4.41 | >4.41 | >4.41 |
| | | | Candida albicans EP | NT | NT | 3.93 | >4.23 | >4.23 |
| | | | Candida albicans USP | NT | NT | 2.95 | >4.23 | >4.23 |
| 8.4 | 25 | 0.24 | Pseudomonas aeruginosa | >4.72 | >4.72 | >4.72 | >4.72 | >4.72 |
| | | | Staphylococcus aureus | 1.32 | >4.64 | >4.64 | >4.64 | >4.64 |
| | | | Escherichia coli | NT | NT | >4.59 | >4.59 | >4.59 |
| | | | Aspergillus hrasiliensis | NT | NT | >4.41 | >4.41 | >4.41 |
| | | | Candida albicans EP | NT | NT | 3.39 | >4.23 | >4.23 |
| | | | Candida albicans USP | NT | NT | 2.63 | >4.23 | >4.23 |
| 8.1 | 10 | 0.24 | Pseudomonas aeruginosa | >4.72 | >4.72 | >4.72 | >4.72 | >4.72 |
| | | | Staphylococcus aureus | 0.76 | 2.89 | >4.64 | >4.64 | >4.64 |
| | | | Escherichia coli | NT | NT | >4.59 | >4.59 | >4.59 |
| | | | Aspergillus hrasiliensis | NT | NT | >4.41 | >4.41 | >4.41 |
| | | | Candida albicans EP | NT | NT | 2.55 | >4.23 | >4.23 |
| | | | Candida albicans USP | NT | NT | 2.00 | >4.23 | >4.23 |
| 8.1 | 15 | 0.26 | Pseudomonas aeruginosa | >4.79 | >4.79 | >4.79 | >4.79 | >4.79 |
| | | | Staphylococcus aureus | 0.30 | 1.52 | >4.88 | >4.88 | >4.88 |
| | | | Escherichia coli | NT | NT | >4.70 | >4.70 | >4.70 |
| | | | Aspergillus hrasiliensis | NT | NT | >4.59 | >4.59 | >4.59 |
| | | | Candida albicans EP | NT | NT | >4.51 | >4.51 | >4.51 |
| | | | Candida albicans USP | NT | NT | >4.51 | >4.51 | >4.51 |
| 8.1 | 20 | 0.26 | Pseudomonas aeruginosa | >4.79 | >4.79 | >4.79 | >4.79 | >4.79 |
| | | | Staphylococcus aureus | 0.34 | 1.58 | >4.88 | >4.88 | >4.88 |
| | | | Escherichia coli | NT | NT | >4.70 | >4.70 | >4.70 |
| | | | Aspergillus hrasiliensis | NT | NT | >4.59 | >4.59 | >4.59 |
| | | | Candida albicans EP | NT | NT | >4.51 | >4.51 | >4.51 |
| | | | Candida albicans USP | NT | NT | >4.51 | >4.51 | >4.51 |
| 7.8 | 10 | 0.27 | Pseudomonas aeruginosa | >4.94 | >4.94 | >4.94 | >4.94 | >4.94 |
| | | | Staphylococcus aureus | 0.75 | 1.09 | >4.89 | >4.89 | >4.89 |
| | | | Escherichia coli | NT | NT | >4.98 | >4.98 | >4.98 |
| | | | Aspergillus hrasiliensis | NT | NT | >4.85 | >4.85 | >4.85 |
| | | | Candida albicans EP | NT | NT | 2.47 | >4.30 | >4.30 |
| | | | Candida albicans USP | NT | NT | 2.47 | >4.30 | >4.30 |
| 8.1 | 17.5 | 0.27 | Pseudomonas aeruginosa | >4.87 | >4.87 | >4.87 | >4.87 | >4.87 |
| | | | Staphylococcus aureus | 1.31 | 3.27 | >4.87 | >4.87 | >4.87 |
| | | | Escherichia coli | NT | NT | >4.76 | >4.76 | >4.76 |
| | | | Aspergillus hrasiliensis | NT | NT | >4.53 | >4.53 | >4.53 |
| | | | Candida albicans EP | NT | NT | >4.40 | >4.40 | >4.40 |
| | | | Candida albicans USP | NT | NT | >4.40 | >4.40 | >4.40 |
| 8.2 | 17.5 | 0.28 | Pseudomonas aeruginosa | >5.04 | >5.04 | >5.04 | >5.04 | >5.04 |
| | | | Staphylococcus aureus | 3.03 | >4.89 | >4.89 | >4.89 | >4.89 |
| | | | Escherichia coli | NT | NT | >4.97 | >4.97 | >4.97 |
| | | | Aspergillus hrasiliensis | NT | NT | >4.59 | >4.59 | >4.59 |
| | | | Candida albicans EP | NT | NT | >4.20 | >4.20 | >4.20 |
| | | | Candida albicans USP | NT | NT | >4.20 | >4.20 | >4.20 |
| 7.8 | 25 | 0.28 | Pseudomonas aeruginosa | >5.04 | >5.04 | >5.04 | >5.04 | >5.04 |
| | | | Staphylococcus aureus | 2.03 | 3.94 | >4.89 | >4.89 | >4.89 |
| | | | Escherichia coli | NT | NT | >4.97 | >4.97 | >4.97 |
| | | | Aspergillus hrasiliensis | NT | NT | >4.59 | >4.59 | >4.59 |
| | | | Candida albicans EP | NT | NT | >4.20 | >4.20 | >4.20 |
| | | | Candida albicans USP | NT | NT | >4.20 | >4.20 | >4.20 |
| 8.4 | 10 | 0.28 | Pseudomonas aeruginosa | >5.04 | >5.04 | >5.04 | >5.04 | >5.04 |
| | | | Staphylococcus aureus | 2.92 | >4.89 | >4.89 | >4.89 | >4.89 |
| | | | Escherichia coli | NT | NT | >4.97 | >4.97 | >4.97 |
| | | | Aspergillus hrasiliensis | NT | NT | >4.59 | >4.59 | >4.59 |
| | | | Candida albicans EP | NT | NT | >4.20 | >4.20 | >4.20 |
| | | | Candida albicans USP | NT | NT | >4.20 | >4.20 | >4.20 |
| 8.1 | 25 | 0.31 | Pseudomonas aeruginosa | >5.04 | >5.04 | >5.04 | >5.04 | >5.04 |
| | | | Staphylococcus aureus | 4.72 | >4.89 | >4.89 | >4.89 | >4.89 |
| | | | Escherichia coli | NT | NT | >4.97 | >4.97 | >4.97 |
| | | | Aspergillus hrasiliensis | NT | NT | >4.59 | >4.59 | >4.59 |
| | | | Candida albicans EP | NT | NT | >4.20 | >4.20 | >4.20 |
| | | | Candida albicans USP | NT | NT | >4.20 | >4.20 | >4.20 |
| 7.8 | 25 | 0.31 | Pseudomonas aeruginosa | >4.87 | >4.87 | >4.87 | >4.87 | >4.87 |
| | | | Staphylococcus aureus | 2.11 | >4.87 | >4.87 | >4.87 | >4.87 |
| | | | Escherichia coli | NT | NT | >4.76 | >4.76 | >4.76 |
| | | | Aspergillus hrasiliensis | NT | NT | >4.53 | >4.53 | >4.53 |
| | | | Candida albicans EP | NT | NT | >4.40 | >4.40 | >4.40 |
| | | | Candida albicans USP | NT | NT | >4.40 | >4.40 | >4.40 |
| 8.4 | 25 | 0.31 | Pseudomonas aeruginosa | >4.79 | >4.79 | >4.79 | >4.79 | >4.79 |
| | | | Staphylococcus aureus | 0.88 | >4.88 | >4.88 | >4.88 | >4.88 |
| | | | Escherichia coli | NT | NT | >4.70 | >4.70 | >4.70 |
| | | | Aspergillus hrasiliensis | NT | NT | >4.59 | >4.59 | >4.59 |
| | | | Candida albicans EP | NT | NT | >4.51 | >4.51 | >4.51 |
| | | | Candida albicans USP | NT | NT | >4.51 | >4.51 | >4.51 |
| 7.8 | 10 | 0.32 | Pseudomonas aeruginosa | >4.94 | >4.94 | >4.94 | >4.94 | >4.94 |
| | | | Staphylococcus aureus | 2.93 | >4.89 | >4.89 | >4.89 | >4.89 |
| | | | Escherichia coli | NT | NT | >4.98 | >4.98 | >4.98 |
| | | | Aspergillus hrasiliensis | NT | NT | >4.85 | >4.85 | >4.85 |
| | | | Candida albicans EP | NT | NT | >4.30 | >4.30 | >4.30 |
| | | | Candida albicans USP | NT | NT | >4.30 | >4.30 | >4.30 |
| 8.4 | 17.5 | 0.32 | Pseudomonas aeruginosa | >4.94 | >4.94 | >4.94 | >4.94 | >4.94 |
| | | | Staphylococcus aureus | 3.85 | >4.89 | >4.89 | >4.89 | >4.89 |
| | | | Escherichia coli | NT | NT | >4.98 | >4.98 | >4.98 |
| | | | Aspergillus hrasiliensis | NT | NT | >4.85 | >4.85 | >4.85 |
| | | | Candida albicans EP | NT | NT | >4.30 | >4.30 | >4.30 |
| | | | Candida albicans USP | NT | NT | >4.30 | >4.30 | >4.30 |
| 7.8 | 10 | 0.33 | Pseudomonas aeruginosa | >4.72 | >4.72 | >4.72 | >4.72 | >4.72 |
| | | | Staphylococcus aureus | >4.64 | >4.64 | >4.64 | >4.64 | >4.64 |
| | | | Escherichia coli | NT | NT | >4.59 | >4.59 | >4.59 |
| | | | Aspergillus hrasiliensis | NT | NT | >4.41 | >4.41 | >4.41 |
| | | | Candida albicans EP | NT | NT | >4.23 | >4.23 | >4.23 |
| | | | Candida albicans USP | NT | NT | >4.23 | >4.23 | >4.23 |
| 8.4 | 10 | 0.34 | Pseudomonas aeruginosa | >4.87 | >4.87 | >4.87 | >4.87 | >4.87 |
| | | | Staphylococcus aureus | 2.51 | >4.87 | >4.87 | >4.87 | >4.87 |
| | | | Escherichia coli | NT | NT | >4.76 | >4.76 | >4.76 |
| | | | Aspergillus hrasiliensis | NT | NT | >4.53 | >4.53 | >4.53 |
| | | | Candida albicans EP | NT | NT | >4.40 | >4.40 | >4.40 |
| | | | Candida albicans USP | NT | NT | >4.40 | >4.40 | >4.40 |

¹NT = Not tested

### Example 23: Controlling Dissolved Oxygen in the Multi-Dose Drug Product to Control HMW Aggregates

An advantageous formulation is 20 mM sodium phosphate, 220 mM sorbitol, 0.31% m-cresol, pH=8.1. The m-cresol is particularly useful to support multi-dose use of MEDI0382 under patient climate conditions. To meet this requirement, MEDI0382 drug product should showcase enough (>3 weeks) in-use stability (at 30° C.) additional to proposed long-term storage condition (at 5° C.). By the end of 24 months at refrigerated conditions and 4 weeks at 30° C., this formulation has ˜5% of HMW impurities using the SEC analytical method.

Exploratory studies highlighted the possibilities of oxidation to form HMW impurities. The MEDI0382 compounding process, which is designed to be performed under normal atmospheric conditions, was reviewed. The normal atmospheric condition has approximately 20% of oxygen along with other components in gaseous phase. This high level of oxygen concentration potentially interacts with MEDI0382 and initiates HMW impurity formation. The standard industry practice highlighted the use of anti-oxidants such as methionine to control oxidation; however, the use of methionine did not control HMW impurity formation in MEDI0382 formulations.

There was no known technique for controlling oxidation of multi-dose peptides during the compounding stage. Therefore, a compounding process for multi-dose peptide formulation was developed by depleting dissolved oxygen content using a novel method. This method utilizes dry nitrogen gas to displace dissolved oxygen present in the multi-dose peptide formulation. The oxygen displacement methodology includes the following stages.

Stage 1: reduced dissolved oxygen multi dose formulation buffer preparation Using a submerged nitrogen tube, dry nitrogen is purged into the multi-dose formulation buffer at steady state conditions for a sufficient period (approximately 30 minutes for 1 liter solution) until the dissolved oxygen content is below 5% of atmospheric content.
Stage 2: formulated drug substance preparation Peptide is added to the reduced dissolved oxygen multi-dose formulation buffer under closed condition and mixed well.
Stage 3: reduced dissolved oxygen multi-dose drug product preparation
The solution is sterile filtered, and the dissolved oxygen content is measured. If the dissolved oxygen content is more than 5%, dry nitrogen is used to displace the excess dissolved oxygen.

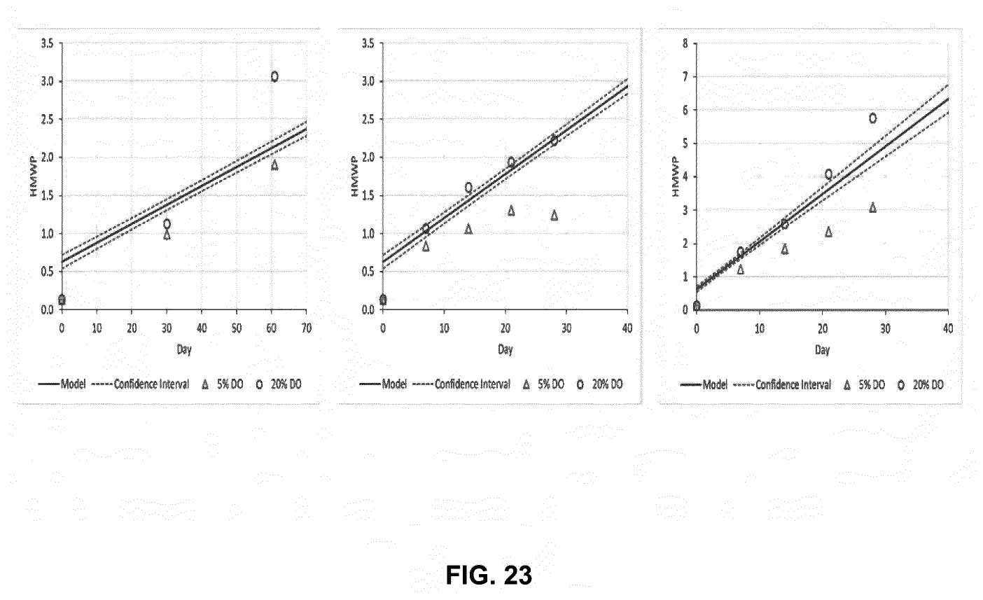

The MEDI0382 drug product produced using this process was subjected to stability studies, and results were compared against the MEDI0382 compounded in normal atmospheric conditions. The results are shown in FIG. 23 and Table 43. The results in Table 43 compare the fitted rate (from 5% DO, 20% DO) to Arrhenius model. The Arrhenius plot uses Log (rate) Vs 1/Temperature.

TABLE 43
Comparison of HMW Degradation
Kinetics (Fitted rate/Arrhenius model)

| Temp (° C.) | 5% DO | 20% DO |
|---|---|---|
| 32 | 67% | 125% |
| 40 | 70% | 136% |

In all study conditions, the MEDI0382 drug product manufactured using the process using 5% dissolved oxygen (DO) had significantly lower HMW impurities (%) than the drug product manufactured in normal atmospheric conditions.

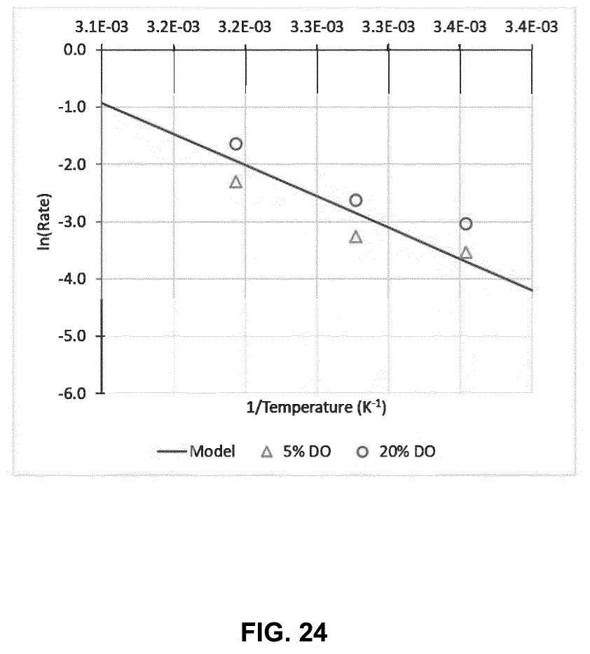

The study results were compared against pre-developed Arrhenius model (developed using DP from earlier manufacturing process). The Arrhenius fit shown in FIG. 24 confirms that MEDI0382 drug product from the process using dissolved oxygen has a statistically significant lower HMW degradation rate than the drug product manufactured in normal atmospheric conditions.

These stability studies and the analysis thereof show that the levels of dissolved oxygen in the multi-dose formulation buffer can be controlled during the compounding stage. This will minimize the MEDI0382 exposure to oxidation and provide a compounding process that consistently manufactures the drug product with low HMW impurities. The drug product from this process has >4 weeks of in-use stability.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A pharmaceutical composition comprising a peptide comprising SEQ ID NO:4, wherein the pH of the composition is about 8.1.

2. A pharmaceutical composition comprising a peptide comprising SEQ ID NO:4 and sorbitol.

3. A pharmaceutical composition comprising a peptide comprising SEQ ID NO:4 and meta-cresol.

4. The pharmaceutical composition of claim 2 or 3, wherein the pH of the composition is at least 7.9.

5. The pharmaceutical composition of claim 2 or 3, wherein the pH of the composition is about 7.9 to about 8.4, optionally wherein the pH of the composition is about 8.1.

6. The pharmaceutical composition of any one of claims 1-3, wherein the composition comprises a pH-adjusting agent.

7. The pharmaceutical composition of any one of claims 1-3, wherein the composition comprises sodium hydroxide.

8. The pharmaceutical composition of claim 2 or 3, wherein the composition comprises sodium hydroxide at a concentration sufficient to make the pH of the composition at least 7.9.

9. The pharmaceutical composition of claim 2 or 3, wherein the composition comprises sodium hydroxide at a concentration sufficient to make the pH of the composition about 7.9 to about 8.4, optionally about 8.1.

10. The pharmaceutical composition of any one of claim 1 or 3-9, wherein the composition comprises a tonicity agent.

11. (canceled)

12. The pharmaceutical composition of claim 2, wherein the concentration of sorbitol is about 190 mM to about 250 mM.

13. (canceled)

14. The pharmaceutical composition of claim 2, wherein the concentration of sorbitol is about 35 mg/mL to about 45 mg/mL.

15. (canceled)

16. The pharmaceutical composition of any one of claim 1 or 2, wherein the composition comprises an antimicrobial agent, optionally wherein the antimicrobial agent is meta-cresol or phenol.

17. The pharmaceutical composition of claim 3, wherein the concentration of meta-cresol is about 0.27% w/v to about 0.45% w/v or wherein the concentration of meta-cresol is about 25 mM to about 30 mM.

18. (canceled)

19. The pharmaceutical composition of claim 3, wherein the concentration of meta-cresol is about 2.7 mg/ml to about 4.5 mg/ml.

20. The pharmaceutical composition of claim 3, wherein the concentration of meta-cresol is about 3.1 mg/ml.

21. The pharmaceutical composition of any one of claims 1-3, wherein the composition comprises a buffer, optionally wherein the buffer is sodium phosphate or TRIS.

22-33. (canceled)

34. The pharmaceutical composition of any one of claims 1-3, wherein the concentration of the peptide comprising SEQ ID NO:4 is about 0.5 mg/mL to about 5 mg/mL.

35-37. (canceled)

38. A pharmaceutical composition comprising about 0.5 mg/mL to about 5 mg/mL of a peptide comprising SEQ ID NO:4, about 190 mM to about 250 mM sorbitol, about 5 mM to about 25 mM sodium phosphate, and about 0.27% w/v to about 0.45% w/v meta-cresol, and wherein the pH of the pharmaceutical composition is about 7.9 to about 8.4.

39. A pharmaceutical composition comprising about 0.5 mg/mL to about 5 mg/mL of a peptide comprising SEQ ID NO:4, about 220.3 mM sorbitol, about 20.1 mM sodium phosphate, and about 0.31% w/v meta-cresol, and wherein the pH of the pharmaceutical composition is about 8.1.

40. A pharmaceutical composition comprising about 0.5 mg/mL to about 5 mg/mL of a peptide comprising SEQ ID NO:4, about 220.3 mM sorbitol, about 20 mM sodium phosphate, and about 0.31% w/v meta-cresol, and wherein the pH of the pharmaceutical composition is about 8.1.

41. (canceled)

42. A pharmaceutical composition comprising about 0.5 mg/mL to about 5 mg/mL of a peptide comprising SEQ ID NO:4, about 220.3 mM sorbitol, about 10 mM sodium phosphate, and about 0.31% w/v meta-cresol, and wherein the pH of the pharmaceutical composition is about 8.1.

43-50. (canceled)

51. A syringe, vial, or a pen comprising the pharmaceutical composition of any one of claims 1-3, optionally wherein the syringe, vial, or pen is a multi-dose syringe, vile, or pen.

52. A method of treating Nonalcoholic Steatohepatitis (NASH) or Nonalcoholic Fatty Liver Disease (NAFLD) comprising administering to a human subject in need thereof the pharmaceutical composition of any one of claims 1-3.

53. A method of reducing liver fat comprising administering to a human subject in need thereof the pharmaceutical composition of any one of claims 1-3.

54. A method of treating type 2 diabetes mellitus comprising administering to a human in need thereof the pharmaceutical composition of any one of claims 1-3.

55-57. (canceled)


SEQUENCE LISTING (4 sequences)

SEQ ID NO: 1 - protein, 29 aa, Homo sapiens
HSQGTFTSDYSKYLDSRRAQDFVQWLMNT

SEQ ID NO: 2 - protein, 30 aa, Homo sapiens
HAEGTFTSDVSSYLEGQAAKEFIAWLVKGR

SEQ ID NO: 3 - protein, 31 aa, Homo sapiens
HAEGTFTSDVSSYLEGQAAKEFIAWLVKGRG

SEQ ID NO: 4 - protein, 30 aa, Artificial Sequence
  chimeric GLP-1/glucagon peptide
  MISC_FEATURE (10)..(10): Lysine with a palmitoyl group conjugated to the epsilon nitrogen,through a gamma glutamic acid linker
HSQGTFTSDKSEYLDSERARDFVAWLEAGG